(19) United States
(12) Patent Application Publication
(10) Pub. No.: US 2026/0041492 A1
(43) Pub. Date: Feb. 12, 2026

(54) THREE-DIMENSIONAL MODEL GENERATION AND SURGICAL PLANNING BASED THEREON

(71) Applicant: Disior Ltd., Englewood, CO (US)

(72) Inventors: Eero HUOTILAINEN, Espoo (FI); Juha TAMPIO, Tampere (FI); Anna-Maria HENELL, Helsinki (FI); Adam Thomas JENSEN, Golden, CO (US); Albert DACOSTA, Englewood, CO (US); Jason Allan EDIE, Sandy, UT (US); Gregory J. KOWALCZYK, Englewood, CO (US); Francis D. BARMES, Parker, CO (US)

(73) Assignee: Disior Ltd., Englewood, CO (US)

(21) Appl. No.: 19/363,055

(22) Filed: Oct. 20, 2025

Related U.S. Application Data
(63) Continuation of application No. PCT/US2024/025157, filed on Apr. 18, 2024.
(60) Provisional application No. 63/497,509, filed on Apr. 21, 2023.
(60) Provisional application No. 63/594,992, filed on Nov. 1, 2023.

(51) Int. Cl. A61B 34/10; G06T 7/00; G06T 17/20; G06T 19/20

(52) U.S. Cl. CPC A61B 34/10; G06T 7/0014; G06T 17/20; G06T 19/20; A61B 2034/104; A61B 2034/105; G06T 2200/04; G06T 2207/30004; G06T 2210/41; G06T 2219/2021

(57) ABSTRACT

Surgical planning based on three-dimensional (3D) models includes obtaining two-dimensional (2D) imaging data of an anatomical region, of a patient, having patient anatomical features, generating a three-dimensional (3D) model of the anatomical region, the 3D model being specific to the patient and providing a 3D representation of the patient anatomical features, identifying at least one deformity exhibited in the 3D model and identified based on the 3D model and relative to target anatomical values for the patient anatomical features, where the identifying includes obtaining anatomical measurements based on anatomical landmarks of the patient as exhibited in the 3D model, comparing the anatomical measurements to the target anatomical values, and determining the at least one deformity based on the comparing, and determining, based on a relationship between the anatomical measurements and the target anatomical values, at least one correction to make to at least one anatomical structure of the patient.

### CROSS-REFERENCE TO RELATED APPLICATIONS

This application is a continuation of International Application Number PCT/US2024/025157, entitled “THREE-DIMENSIONAL MODEL GENERATION AND SURGICAL PLANNING BASED THEREON”, filed Apr. 18, 2024, and published Oct. 24, 2024 as International Publication Number WO 2024/220642 which is hereby incorporated by reference in its entirety, and which claims the benefit of U.S. Provisional Application Nos. 63/497,509 filed Apr. 21, 2023 and 63/594,992 filed Nov. 1, 2023, both entitled “THREE-DIMENSIONAL MODEL GENERATION AND SURGICAL PLANNING BASED THEREON”.

### BACKGROUND

Software is increasingly being used in the diagnosis of patient medical conditions and surgical planning to address those conditions. A foundation of this activity is an accurate reflection and understanding of the patient's anatomical properties, which is often gleaned from patient image data.

### SUMMARY

Shortcomings of the prior art are overcome and additional advantages are provided. As one example, a method is provided that includes obtaining a three-dimensional (3D) model of an anatomical region of a patient, the anatomical region comprising patient anatomical features. The method further includes identifying at least one deformity of the patient anatomical features, wherein the at least one deformity is identified relative to target anatomical values for the patient anatomical features, wherein the target anatomical values comprise desired ranges into which anatomical measurements are to fall, and wherein the at least one deformity comprises at least one of a rotational deformity and an angular deformity. The method additionally includes determining, using the anatomical measurements, at least one planar correction to make to at least one anatomical structure of the patient.

As another example, a method is provided that includes obtaining two-dimensional (2D) imaging data of an anatomical region of a patient, the anatomical region comprising patient anatomical features. The method further includes generating, using the 2D imaging data, a three-dimensional (3D) model of the anatomical region of the patient, the 3D model being specific to the patient and providing a 3D representation of the patient anatomical features. The method additionally includes identifying one or more deformities of the patient anatomical features, wherein the one or more deformities are exhibited in the 3D model, and are identified based on the 3D model and relative to target anatomical values for the patient anatomical features. The identifying includes obtaining anatomical measurements based on anatomical landmarks of the patient, as exhibited in the 3D model, comparing the anatomical measurements to the target anatomical values, determining the one or more deformities based on the comparing. The method also includes determining, based on a relationship between the anatomical measurements and the target anatomical values, at least one correction to make to at least one anatomical structure of the patient.

As yet another example, a method is provided that includes obtaining two-dimensional (2D) imaging data of an anatomical region of a patient, the anatomical region comprising patient anatomical features. The method also includes generating, using the 2D imaging data, a three-dimensional (3D) model of the anatomical region of the patient, the 3D model being specific to the patient and providing a 3D representation of the patient anatomical features. The generating includes performing anatomical context processing, the anatomical context processing comprising annotating, using an artificial-intelligence (AI) model, contours, edges, or surfaces of anatomical structures, of the anatomical region, presented in the 2D imaging data. The generating also includes performing 2D-to-3D reconstruction that optimizes orientation, placement, and scale of 3D digital volumes modeling the anatomical structures to yield, as the 3D model, a 3D anatomical representation of the anatomical region.

As a further example, a method is provided that includes obtaining two-dimensional (2D) imaging data of an anatomical region of a patient, the anatomical region comprising patient anatomical features. The method also includes generating, using the 2D imaging data, a three-dimensional (3D) model of the anatomical region of the patient, the 3D model being specific to the patient and providing a 3D representation of the patient anatomical features. The method further includes identifying one or more deformities of the patient anatomical features, wherein the one or more deformities are exhibited in the 3D model, and are identified based on the 3D model and relative to target anatomical values for the patient anatomical features. The identifying includes obtaining anatomical measurements based on anatomical landmarks of the patient, as exhibited in the 3D model, comparing the anatomical measurements to the target anatomical values, and determining the one or more deformities based on the comparing. The method also includes determining, based on a relationship between the anatomical measurements and the target anatomical values, at least one correction to make to at least one anatomical structure of the patient. The method additionally includes generating a report comprising instructions for engaging a surgical instrument with the at least one anatomical structure of the patient and manipulating said surgical instrument to perform the at least one correction to the at least one anatomical structure of the patient.

Additional aspects of the present disclosure are directed to systems and computer program products configured to perform the methods described above and herein. The present summary is not intended to illustrate each aspect of, every implementation of, and/or every embodiment of the present disclosure. Additional features and advantages are realized through the concepts described herein.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIG. 1 depicts an example process for surgical planning, in accordance with aspects described herein;

FIG. 2 depicts an example workflow of a method used in surgical planning, in accordance with aspects described herein;

FIG. 3 depicts example inputs and outputs of an AI model for anatomical context processing, in accordance with aspects described herein;

FIG. 4 depicts an example distance transform Di of a binary edge map, in accordance with aspects described herein;

FIGS. 5 and 6 depict examples of plane transformations to reach an optimal solution for 3D modeled anatomical structures of a patient, in accordance with aspects described herein;

FIG. 7 depicts an example conceptual process of 2D-to-3D reconstruction activity, in accordance with aspects described herein;

FIGS. 8 and 9 depict example representations of patient anatomy before and after a Lapidus procedure;

FIGS. 10A-10C depict example Lapidus procedure corrections;

FIG. 11 depicts example normative measures of interest for Lapidus and Hallux correction procedures;

FIGS. 12A-12C depict example osteotomy locations for a Lapidus arthrodesis;

FIGS. 13A and 13B depict example visualizations in Lapidus procedure planning, in accordance with aspects described herein;

FIG. 14 depicts an example process for anatomical correction, in accordance with aspects described herein;

FIG. 15 depicts an example process for a rotational correction, in accordance with aspects described herein;

FIGS. 16A-16C provide example depictions of a barrel geometry applied to a distal-most portion of a first metatarsal, in accordance with aspects described herein;

FIG. 17-20 depict example testing results based on intermetatarsal angles;

FIGS. 21 and 22 depict example virtual correction output relative to a Lapidus procedure and metatarsus adductus correction, in accordance with aspects described herein;

FIGS. 23-24 depict example processes for simulated WBCT model generation, in accordance with aspects described herein;

FIG. 25 depicts an example overall workflow for surgical planning in accordance with aspects described herein;

FIG. 26 presents an example interface for user authentication, in accordance with aspects described herein;

FIGS. 27-29 depict example interfaces for starting a new case for surgical planning, in accordance with aspects described herein;

FIG. 30 depicts an example interface for image type and laterality selection, in accordance with aspects described herein;

FIG. 31 depicts an example interface for a user to confirm (or reject) pre-operative anatomical position and measurement axis/axes, in accordance with aspects described herein;

FIG. 32 depicts an example interface to select whether to proceed with Lapidus procedure planning when a Metatarsus Adductus condition is identified, in accordance with aspects described herein;

FIG. 33 depicts an example interface to select whether to correct for a Metatarsus Adductus condition as part of Lapidus procedure planning, in accordance with aspects described herein;

FIG. 34 depicts an example interface presenting a correction preview, in accordance with aspects described herein;

FIG. 35 depicts an example interface for updating target values, in accordance with aspects described herein;

FIG. 36 depicts an example report view/save interface, in accordance with aspects described herein;

FIG. 37 shown an example summary page of a report, in accordance with aspects described herein;

FIGS. 38A-38D depict example error messages that may be presented to a user of the software;

FIGS. 39A-39C depict example warning messages that may be presented to a user of the software;

FIG. 40 illustrates example bone models of bones in a foot and ankle region, in accordance with aspects described herein;

FIGS. 41A-41D depict example axes defined for bones in a foot and ankle region, in accordance with aspects described herein;

FIGS. 42A-42N illustrate example geometric measurements determined by the software relative to a foot and ankle region, in accordance with aspects described herein;

FIG. 43 depicts a conceptual diagram of example secure communication (via HTTPS) between a user/client and cloud service(s) hosted on a cloud computing platform, in accordance with aspects described herein;

FIG. 44 depicts one example of a computer system and associated devices to incorporate and/or use aspects described herein; and

FIG. 45 depicts an example diagnostic report generated in accordance with aspects described herein.

### DETAILED DESCRIPTION

Three-dimensional (3D) digital models are often valuable representations of patient anatomy for diagnostic and treatment activity. A process might create 3D digital model(s) from patient imaging data and then, from the 3D digital model(s), identify various information. For instance, the process might identify different anatomic landmarks, such as bones and properties thereof (e.g., axes, etc.), and, using those landmarks, identify deformities that might be present. The process then might identify corrections, for instance corrected positions, adjustments, resections, or the like, for the anatomical components, in order to address the deformities.

In accordance with some aspects described herein, patient imaging data is used to construct 3D digital models of patient anatomy. In accordance with some aspects, the patient imaging data is two-dimensional (2D) imaging data (“images”), such as radiographs produced from ionizing radiation (e.g., x-rays, gamma rays). From the 2D images, aspects can generate 3D digital model(s). In particular embodiments, the 2D images consist of multiple images, sometimes as few as just two x-rays, providing different views to an anatomical region of a patient. Even more particularly, in some embodiments, a top x-ray view and a medial or lateral sagittal plane view of an anatomy is obtained as two 2D images, and a process uses these two 2D images to construct a 3D model that can then be used to determine anatomical measurements, identify a deformity for which to provide corrections, and determine such corrections. Aspects may be performed in whole or in part by a surgical planning tool implemented as software executing on computer system(s). Thus, one or more computer systems of a computing environment can incorporate, perform, and/or use aspect(s) described herein.

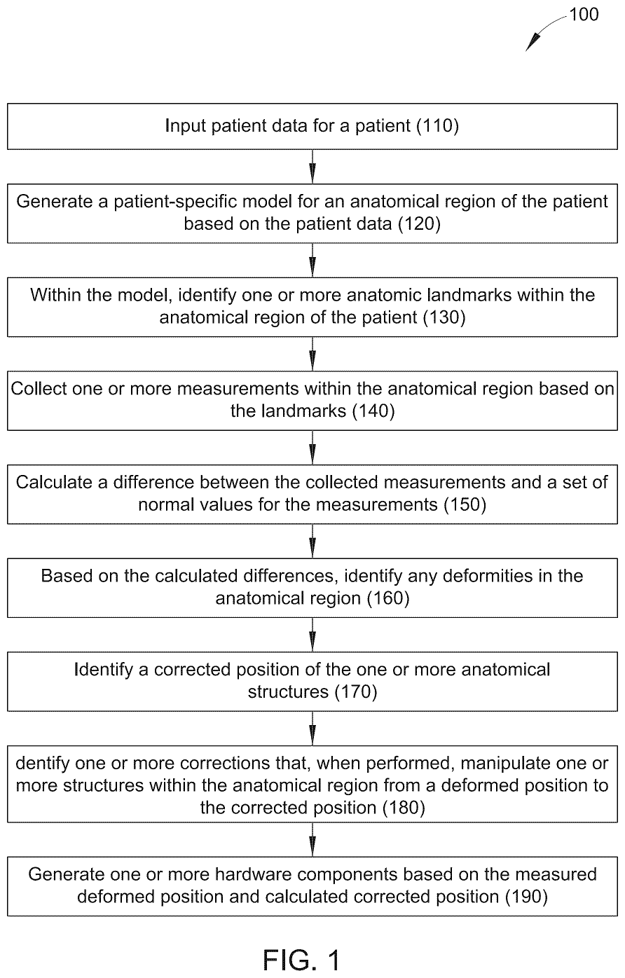

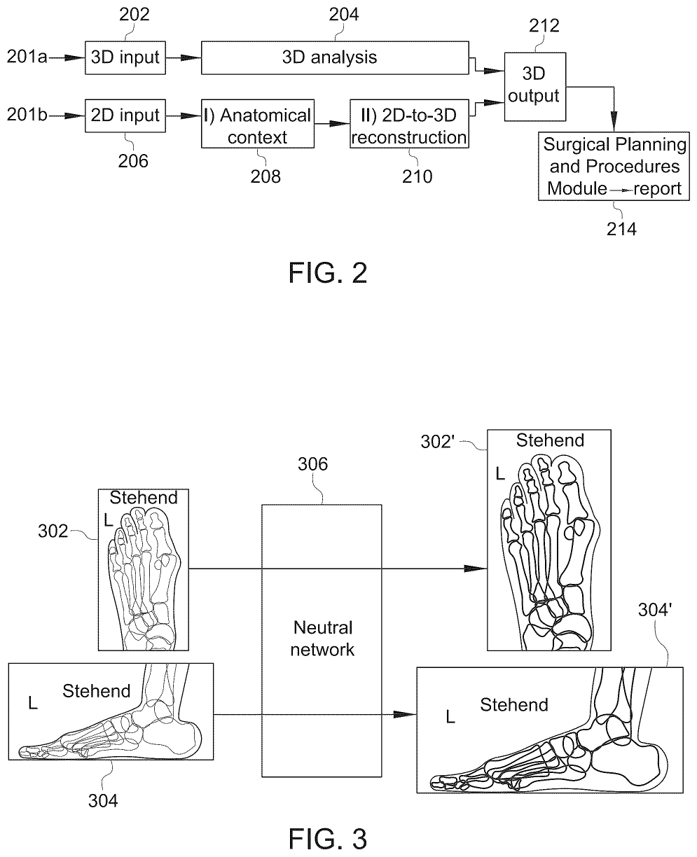

In accordance with some aspects described herein, methods are provided to facilitate surgical planning. Such methods can incorporate 3D model generation and use. Initially, aspects are described with reference to FIG. 1, depicting an example process for surgical planning in accordance with aspects described herein, and FIG. 2, depicting an example workflow used in surgical planning in accordance with aspects described herein.

Initially referring to FIG. 2, the workflow can proceed under one of two paths depending on the particular patient imaging data that is available/input. Under a first path, 201a, the workflow receives 3D patient imaging data input 202 and proceeds with 3D analysis 204 to produce a 3D output 212. The 3D output refers to 3D model(s) of patient anatomy. Under a second path, 201b, the method receives 2D patient imaging data input 206 and proceeds with anatomical context processing 208 followed by 2D-to-3D reconstruction processing 210 to produce 3D output 212. In either scenario, the 3D output 212 is used in a next phase by a surgical planning and procedures module 214 to perform surgical planning and procedure determination, and then generate a report. Additional details of the workflow of FIG. 2 are described with reference to FIG. 1.

Aspects of the process of FIG. 1 can be performed by one or more computer systems, for instance computer(s) that execute program code to perform features described herein. Optionally, some aspects of FIG. 1 (for instance 110) might be performed manually by a user, though it is understood that such manual action by a user might have a corresponding action performed by the computer system (e.g., in the example of 110, the computer system would receive the input patient data).

The process 100 of FIG. 1 includes input (110) of patient data for a patient. By way of example and not limitation, the input patient data could include one or more of the following:
Imaging data, such as x-rays from multiple views, magnetic resonance imaging (MRI) images, computed tomography (CT) images, weight-bearing CT images, etc. of patient anatomy. In examples, imaging data may be in the form of digital imaging and communication and medicine (DICOM) images of patient anatomy;
Data from optical scanners (e.g., to capture properties/features of skin and other anatomy;
Data from pressure sensors (e.g., force plates);
Tracking data (e.g., from optical or magnetic trackers); and
other data of/about the patient, such as age, weight, height, shoe size, measurements of foot or other anatomy, DICOM data, etc.

More generally, any information about/describing patient features might be useful and therefore obtained as part of the patient data. The patient features might be features that can be compared against determined ‘normal’ or other reference features, such as features of a general or specific population of other individuals.

The process proceeds by generating (120) a patient-specific model, for instance as 3D output 212 of FIG. 2, for (at least) one anatomical region of the patient based on the input patient data. The generating (120) incorporates/uses at least some of the patient data provided as input at 110.

In examples where the input patient data includes imaging data, the generating (120) might proceed under one of two paths, and more specifically the paths 201a and 201b of FIG. 2. Referring initially to path 201a, the patient data input at 110 would include 3D image data and the generating (120) takes the 3D image data input and performs 3D analysis 204 (such as segmentation, registration, fitting, etc.) to produce the 3D output 212. 3D analysis 204 can include, in examples, use of an artificial-intelligence (AI) based model, such as a trained neural network, for instance an artificial neural network (ANN), and even more particularly, as an example, a convolutional neural network (CNN). The AI model can be configured to intake the 3D image data/images of a patient and that are reflective of at least an anatomical region (the images depicting an area as large as a whole patient body or as small as any portion of the patient body), produce a rough segmentation of the image(s), identify locations and properties of anatomical features, which could include anatomical structures present in the anatomical region, for instance bone(s), and use that information to generate an initial approximation of a 3D model of the anatomical region with proper position and orientation of the anatomical structures. The 3D analysis can process the model in different stages to provide the anatomical structures with the proper size, in the proper pose, and with proper shapes (e.g., with proper contours on bone anatomy) so that the 3D model accurately, and in detail, reflects correct patient anatomy. In some embodiments, 3D analysis 204 includes repeatedly measuring normal (i.e., at 90 degrees) distances between bone models as the model shape evolves toward the patient-specific shape, which serves dual purposes of preventing overlap between bone models (and therefore physically infeasible bone shapes) and filtering out scanned bone outer cortical surfaces that would result in overlap. The generated 3D model can be output as 3D output 212.

In examples where the input patient data includes 2D imaging data and, in some embodiments, does not include any 3D image data, the generating (FIG. 1 #120) proceeds under path 201b (FIG. 2), with 2D image data being input (206), followed by anatomical context processing 208 and 2D-to-3D reconstruction processing 210 to produce 3D output 212.

The anatomical context processing 208 uses 2D input, including, for instance, image(s)/image data of two or more views (i.e., different angles) of a patient anatomical region. In the context of planning for a Lapidus procedure as described elsewhere herein, the 2D inputs may be (i) top (dorsal plantar) view and (ii) medial or lateral (e.g., sagittal plane) view x-rays of the patient foot, as examples. The anatomical context processing 208 undertakes an annotating aspect described herein, which can involve a trained ANN, for instance a trained CNN, to process the image data and identify contours, edges, surfaces, etc. of anatomical structures, for instance of bones depicted wholly or partially by the image data. The CNN may be akin to the CNN described above with reference to path 201 a except that the CNN performs image segmentation in two dimensions to identify, e.g., locations of contours of patient anatomy. In this regard, the anatomy being identified is often at least partially overlapping and therefore such identification is impossible for a user to perform manually/mentally. The trained AI model (e.g., CNN) assists in this regard, and the analytical context processing/annotating may be done automatically, i.e., absent/without manual/user input. User involvement in this may be absent. It is noted, however, that user input may contribute to the training of the AI model prior to its use/application in the anatomical context processing. For instance, during a development stage of the AI model, a user might provide annotations in the generation of a collection of training data from which the AI model (e.g., network) learns. A goal of proper AI model training may be for the AI model to learn the annotation process and provide correct output, i.e., annotations to image data that identify properties of anatomical structures, without any user input.

Accordingly, one aspect of the processing (208 of FIG. 2) uses a pre-trained ANN, for instance one trained using the U-Net architecture available at https://arxiv.org/abs/1505.04597, or a modified version thereof. In a specific example of a training process, a collection of several hundred manually-annotated 2D-segmented images (dorsoplantar, lateral, and oblique X-rays for instance) are obtained. Some such obtained images (“samples”) may be bilateral images that can be split into unilateral versions to increase the count of samples in the collection. More broadly speaking, it may be desired to produce a sufficiently large collection of sample structures containing a plane of symmetry. The training process can thus augment the collection to further increase the total sample count, for instance by replicating samples by adding rotations, flips, translations, and/or other image manipulations. After this further augmentation, the collection can then be divided into subsets, for instance (i) a collection of training samples to train the model to an initial fit, (ii) a collection of validation samples against which the fitted model is used to predict responses, for instance for purposes of avoiding overfitting, and (iii) a collection of test samples as a separate set used to assess network performance.

The AI model is thereby trained to take 2D images (2D image data) as input and process the input to determine/generate/provide output by way of neural network inference. Output can be or include, as an example, a binary segmentation mask for each of multiple anatomical structures, for instance bones of an anatomical region depicted by the images. By way of example, in the case of a foot and ankle region, the AI model may look to identify approximately 30 bones. The 2D segmentations can then be processed into a set of 2D points by computing (i) a centroid of each detected anatomical structure (e.g., bone), and (ii) certain other definable landmarks, such as, in the case of a patient foot, proximal and distal ends of metatarsals and proximal phalanges. The neural network inferencing can be called separately for each input 2D image.

FIG. 3 depicts example inputs and outputs of an AI model for anatomical context processing, in accordance with aspects described herein. Input x-ray image 302 and image 304 are top and sagittal plane views, respectively, of a patient foot, and are inputs to AI model 306. Outputs of the model 306 are annotated versions of image 302 and image 304. Thus, image 302′ is an annotated version of image 302 and image 304′ is an annotated version of image 304. The annotations in these examples include outlines (indications of the peripheries) of identified bones of the patient foot. All visible bones are shown contoured on the 2D image plane with outlines of different bones being provided in different colors to aide identification and understanding. It is seen there is significant overlap in many of the bones that were identified. In examples, this annotated image data is displayed to a user on display device(s) of, or in communication with, a computer system. Other annotations and ways of identifying/distinguishing anatomy are possible.

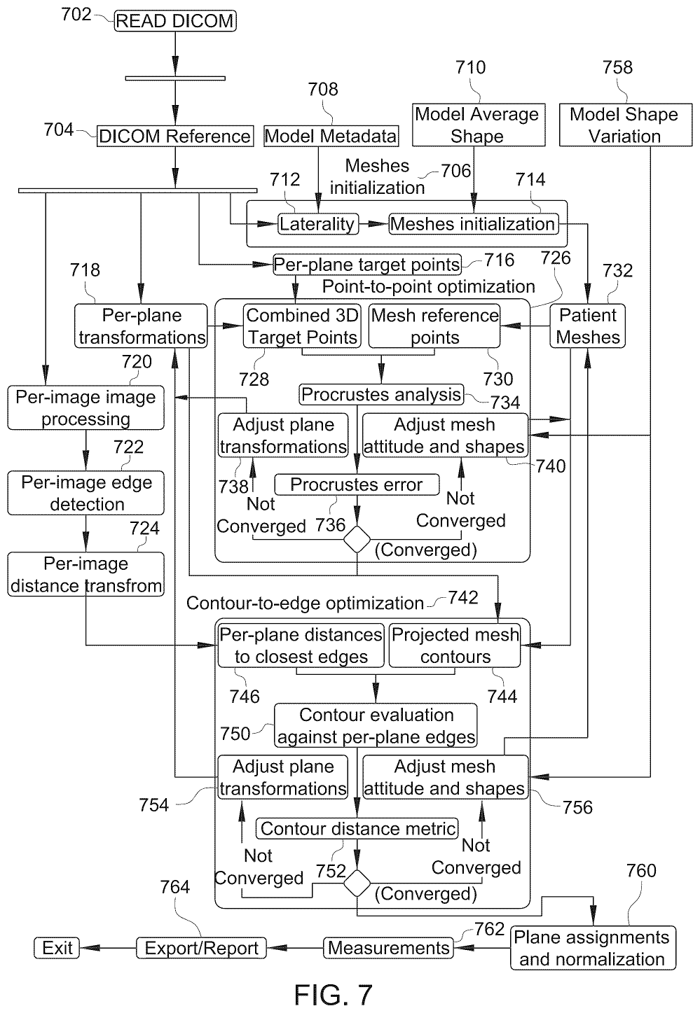

The anatomical context processing 208 provides the annotated image data, after which the workflow proceeds with the 2D-to-3D reconstruction at 210 of FIG. 2 (part of the generating the 3D model at 120 of FIG. 1) using the annotated image data. In the reconstruction 210, 3D image data/image(s) is/are constructed using the annotated image data. FIG. 7 depicts an example conceptual process of 2D-to-3D reconstruction activity, in accordance with aspects described herein. In the example of FIG. 3, there are two annotated x-ray images corresponding to two different views.

In some embodiments, the centroids of the contours are used in the reconstruction 210, though it is possible that other features of the annotations—the color-coded outlined bones here-could be used if desired. For example, the reconstruction includes processing that finds, for each annotated anatomical structure, i.e., bone in these examples, a centroid/center-point of the delineated area. The centroids of the anatomical structures may then be used in an optimization loop to roughly align planes with reference geometries (e.g., normal or average, for instance) for the anatomical structures, for instance from a whole-foot set of 3D models. In this aspect, the process can optimize, for each plane, its orientation, its in-plane translation (disregarding the arbitrary depth component of a projection), and its uniform scale factor. A goal may be to define commensurate points (‘point-pairs’) between certain planar features on the image data (e.g., X-rays) and 3D features on the reference geometries (e.g., 3D anatomical structure). Centroids (center-points) are one option as explained above. Based on the definition of these point-pairs, a process can then perform optimizations while measuring correspondence of the point-pairs as a fitness function, for instance.

In the different planes where there are point(s) representing the bone center-points, a ‘golden model’ may be used with statistical methods to fit the planes together in three-dimensional space, optimizing (though rotations and scaling—“transformations”) how the planes sit relative to each other and how the planes sit relative to the current best estimate of the whole 3D anatomy that is being processed, to provide an accurate anatomical representation of the anatomical region. The ‘golden model’ is, for instance, a model that has average or other ‘normal’ structures (size, shape, orientation, etc.) for that anatomical region—a human foot in this example. The golden model can also have encoded therein certain information about how the anatomy might be expected to vary—for instance variation in pose, individual bone shape, etc.—across a patient population.

The plane transformations, model shapes, and anatomical pose, referring to shape alignments with respect to each other, may be optimized in an iterative fashion. Planar centroids (from the image data/X-rays), for instance, can be compared to 3D centroids (center-points of actual 3D models), or more broadly, any defined commensurate point-pairs (2D feature correlated to 3D feature) can be compared. Additionally, ‘edges’ of the 3D models may be compared against edges (e.g., gradient boundaries) of the imaging data (taking into account the fact that the X-ray image data is on plane, the transformation of which is being iteratively estimated). In this manner, parameters are found for each of the planes while modifying anatomical pose. The planes are expected to converge generally toward the golden model, but the 2D image data informs, via image processing techniques such as edge detection and gradient spikes in the images, tweaking to the plane transformations to be consistent with what the actual 2D image data expresses. For instance, with respect to bone surfaces, an approach may be to look for cortical bone surfaces that are nearly perpendicular to the projection (X-ray view) direction. At such features, it can be expected to see gradient drops/spikes in X-ray signals. An edge detection method at various sensitivity levels can be used in order to capture both relatively faint edges on low-signal/low-contrast images while also being able to incentivize the optimization loop to find the relatively sharper/stronger edges on images, where more ‘false positives’ would occur. The equivalent for the X-ray contours on the 3D models are defined as the subset of model vertices (mesh points) where the surface normal is sufficiently perpendicular to the projection (view) vector.

An example of shape modeling in this fashion is provided using statistical shape modeling. In a particular embodiment, a pre-defined statistical pose and shape model (′SSM′, which is also used herein as an abbreviation to refer to the technique of statistical shape modeling using such a model) is obtained as model metadata 708, model average shape 710, and model shape variation 758 inputs of FIG. 7. The SSM may be generated based on a population of varying segmented anatomies that correspond to those anatomies to be modeled from given input patient images. Since the model may be tailored to particular anatomies/anatomical regions, there may be a selection of the SSM from a collection of different available such SSMs configured for different anatomies/anatomical regions.

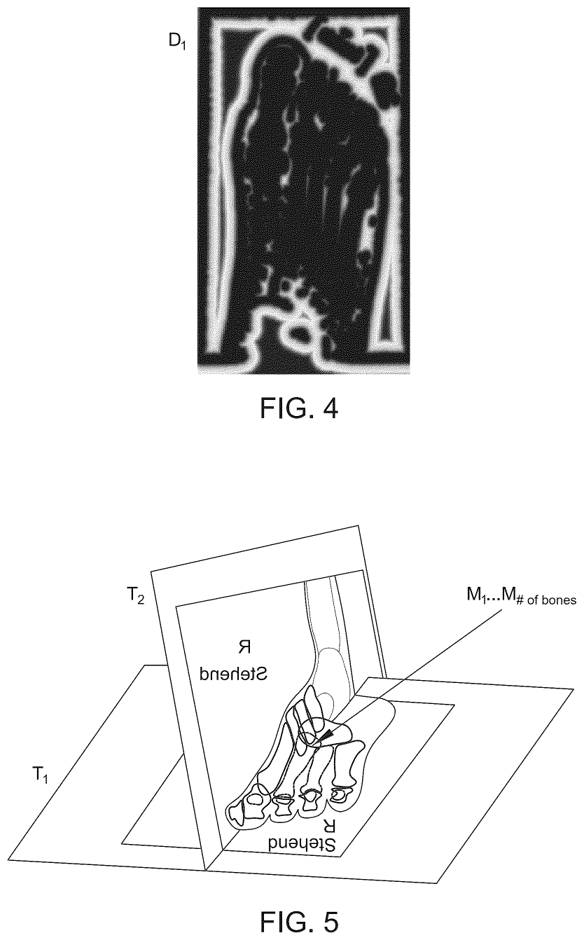

With the appropriate SSM obtained, the reconstruction can proceed against each of the annotated input images (read from DICOM data 702 as DICOM reference 704 input). For each such 2D image i (i=1, . . . , number of input 2D images), the process:
a) has the set of target points pᵢ (as planar X-Y coordinates on the 2D images) (716 of FIG. 7);
b) runs the following per-image image processing (720 of FIG. 7) steps:
   (i) detects image edges of individual anatomical features that are at least partially shown in the image (722 of FIG. 7) using a gradient-magnitude based method such as Sobel filtering or Canny method, as examples (image processing methods may look at the raw image data without any anatomical context but are expected to correlate to certain anatomical features of interest); and
   (ii) computes distance transform (724 of FIG. 7) Dᵢ of the binary edge map (see FIG. 4 for an example such distance transform Dᵢ of a binary edge map, in accordance with aspects described herein); and
c) initializes a 3D plane carrying (initially arbitrary) transformation Tᵢ representing:
   (i) 3 degrees-of-freedom for the plane orientation;
   (ii) 2 degrees-of-freedom for planar translation components (i.e., not three dimensions as the “depth” component of a projection view is arbitrary/undefined); and
   (iii) 1 degree-of-freedom for global scale factor of the plane.

The initialization can be an initialization of the per-plane transformations (718), referring to a collection of transformation parameters that may be tweaked (at 738, 754) and improved upon during the optimization. One example of such initialization is an assignment of arbitrary values for these parameters.

The per-plane target points 716 provides an anatomical context for the optimization, and can be based on a determination of anatomical structures (such as bones) contained within the field-of-view of the given image. Such determination could be made manually, automatically, or semi-automatically, and using any desired approach. In examples, this is done as a subprocess. In embodiments as described, an AI-based model, for instance an artificial neural network (ANN) is used. More specifically, the model may be trained to identify certain bones (or other anatomical structures) and segment them (e.g., trace their boundaries) on the 2D X-ray images. This identifies structures that do, and do not, exist in the image, provides rough estimates of where structures existing in the image are situated, and can be used for ‘point-to-point’ optimization. An advantage of an AI-model based approach is that this aspect could be fully automated.

Then, for each anatomical structure (such as bone) to analyze (j=1, . . . , number of anatomical structures), the process proceeds with meshes initialization (706 of FIG. 7) which initializes (714 of FIG. 7) a 3D model Mⱼ (sometimes termed a patient mesh, 732 of FIG. 7) based on laterality (712 of FIG. 7) by, for instance, retrieving, e.g., from model metadata 708 of FIG. 7 and model average shape 710 of FIG. 7, a mean pose and anatomical structure (e.g., bone) shape combination from the SSM. The SSM provides a baseline or ‘normal’ anatomy for each anatomical structure (e.g., bone) from which a proper model for said structure exhibited in the patient anatomy is eventually derived, e.g., by imparting modifications to the baseline until the baseline has been sufficiently modified to match the indicated patient anatomy as reflected by the images/scans. Starting with a mean shape (710 of FIG. 7) from the SSM, the process can make any number of modifications (740, 756 of FIG. 7) to the mean shape. The SSM can also encompass data relating to the variation of the shape, for example how it varies and how large the variation is from the normal (indicated by variation input 758). In this manner, the statistical shape model can inform (a) average shape (710 of FIG. 7) and (b) how the average shape varies (758 of FIG. 7) over a certain population, and a process can iterate over those until a sufficiently good match to patient anatomy as reflected by the input images is found.

By way of a specific example, a process runs (executes) optimization loops that iteratively optimize each component of 7, of each plane (718 of FIG. 7—per-plane transformations), each plane correlating to an input image, and each structure model Mⱼ (732 of FIG. 7) by retrieving meaningful pose and shape variation to test from the SSM. An example optimization loop is a point-to-point optimization loop (726 of FIG. 7), in which (i) for each plane, 3D target point set Pᵢ (728 of FIG. 7) is defined by combining each pᵢ (e.g., of 716) and current best estimates for each Tᵢ, and then (ii) Procrustes analysis (734 of FIG. 7) is used to find a rigid transformation minimizing sum of squared differences between Pᵢ and its according 3D reference points (730 of FIG. 7) on the current best estimates of Mⱼ (i.e., from 732 of FIG. 7). Each plane is optimized individually in this aspect (using the Procrustes technique, for instance). After these optimizations, the aggregated result, for instance a sum of squares of per-plane errors, is taken to check whether an improvement results in the global solution. If so, then the updates are made to the planes and meshes (and optionally there may be product-specific constraints that might prohibit certain impossible relative spatial configurations for the planes). Thus, in each iteration, the solution with the smallest Procrustes error (736 of FIG. 7) can be selected and iteratively refined in accordance with the above via (i) plane transformation adjustments (738 of FIG. 7) to produce per-plane transformations (718 of FIG. 7) and (ii) mesh attitude (i.e., pose) and shape adjustments (740 of FIG. 7) informed by model shape variation data (758 of FIG. 7) to produce updated patient meshes (732 of FIG. 7). At each iteration, there may be some selection in which certain updates are discarded if they would appear to increase the recorded error metrics. At the end of each iteration, there is a determination whether the error metric is satisfactorily low (i.e., a determination whether converged). If not, the iterating continues, otherwise the process proceeds with contour-to-edge optimization.

In embodiments, the optimization uses a multiscale iterative technique, though other options may be available, for instance one utilizing the Levenberg-Marquardt algorithm.

Another example optimization loop is a contour-to-edge optimization loop (742 of FIG. 7), in which, given the current best estimates for each Mⱼ (from 732 of FIG. 7) and Tᵢ (from 718 of FIG. 7), the process thresholds, based on vertex-normal directions, a contour set (744 of FIG. 7) of each patient mesh (3D model, Mⱼ), projects it onto each plane, and then evaluates (750 of FIG. 7) its accordance to Dᵢ as contour distance metric (752 of FIG. 7), i.e., assumes that the closer it reaches the optimal global solution, the closer each projected contour point will be to the closest detected edge (from 746 of FIG. 7) on the 2D input image. The contour-to-edge optimization 742 can iterate as above via (i) plane transformation adjustments (754 of FIG. 7) to produce per-plane transformations (718 of FIG. 7) and (ii) mesh attitude (i.e., pose) and shape adjustments (756 of FIG. 7) informed by model shape variation data (758 of FIG. 7) to produce updated patient meshes (732 of FIG. 7).

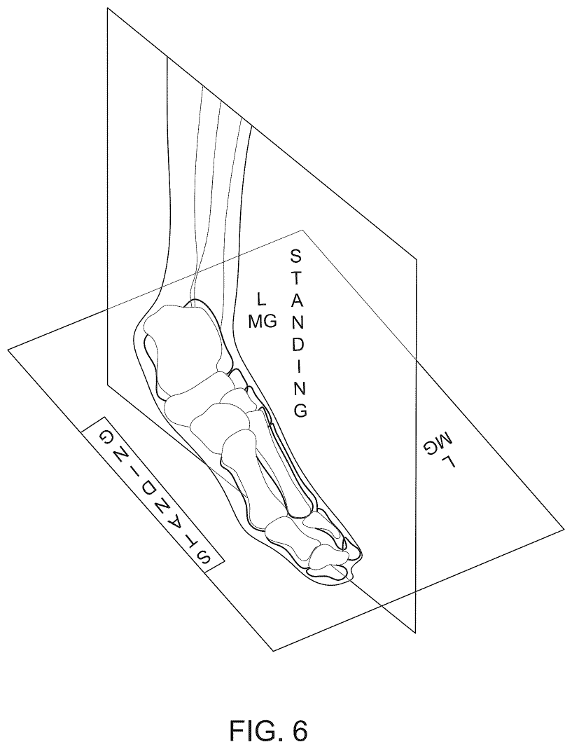

FIGS. 5 and 6 depict examples of plane transformations to reach an optimal solution for 3D modeled anatomical structures of a patient. In particular, FIG. 5 shows an intermediate or final combination of transformations for a plurality (in this case, 2) of planar images against an optimized shape model. Green contours depict planar projections from the 3D models visible in blue, projected onto both of the 2D input images. FIG. 6 is similar to FIG. 5 but with opaque mesh surfaces and more realistic lighting. These examples could be rendered in any desired fashion, for instance as 3D models using triangular meshes, or as point clouds or other non-mesh representations. Aspects of the reconstruction described above therefore estimate both the spatial configuration for all of the input planes (how they sit against the 3D models), as well as the shapes, pose, etc. of the 3D models depicting the anatomy.

Thus, in accordance with aspects of the above, a statistical shape modeling technique may be used in generating a 3D model from 2D image data input. This may be useful in situations where there is limited input image data available, for instance only x-ray (or other 2D image) data input with limited views, and in some examples as few as just two views. While a process can identify specific features on these projective planes/images, a significant amount of information informing a true 3D representation of the anatomy is not available, and therefore it is desired to estimate that information. In examples, the anatomy of a specific population is used to define a model (an SSM) that describes what may be expected, 3-dimensionally, on average in that given population, and therefore the SSM could reflect ‘normal’ anatomy and/or anatomy representing condition(s) of varying degrees of severity. The 2D image data therefore provides a subset of the 3D information that is needed to generate the 3D representation/model of specific patient anatomy. The 2D image data (e.g., input projective images containing the subset of information) is analyzed to identify features that serve as anchors from which to work to determine the rest of the information needed to build the patient anatomy 3D model, i.e., to inform variations in the SSM model to try. Specifically, the process uses the SSM (model) as the source from which to sample information to determine the missing 3D features that are not seen from the projective images. The SSM can encode a base (an example of which is an average or normal) shape/pose of specific anatomy, such as bone(s), in a subject population, as well as quantify how the base varies within that population. In embodiments, the base SSM can be modified toward what is observed as features from the projective planes taken of the patient, and then the missing information can be interpolated from the SSM. In this manner, the features exhibited from the projective images drive the extraction of relevant information from the SSM model to roughly meaningful positions.

As noted, the SSM model can include/indicate variations from a base, for instance variations that correspond to patient conditions. Example such conditions discussed specifically herein include hallux valgus and metatarsus adductus. In any case, parameters of the statistical shape modeling processing can be used to weight the importance of condition-specific variations/poses for use in the statistical shape modeling processing. That is, the SSM processing can be guided by emphasizing variations/deviations from a base, normal, or average to explore and correctly model the anatomy of a specific patient. These variations can correspond to specific conditions. The processing therefore utilizes a combination of parameters that approximate a base or average patient anatomy, and this might be with or without given condition(s) (such as a hallux valgus foot).

Additionally or alternatively, it might be desired to examine what is reflected by the 2D image data of the specific patient, identify characteristics about the patient or a patient population into which the specific patient best fits, and use this information to inform certain model parameters to facilitate the generation of the 3D model. For instance, what is seen in the input data about the patient anatomy could be used to identify a certain population and/or associated combination or model parameters—for instance a population with hallux valgus and/or metatarsus adductus, as examples—This can be used to inform a combination of model parameters to apply to an average-population SSM to help generate a 3D model data of the specific patient anatomy. As another option, various base SSM models could be maintained that correspond to different patient populations, and the characteristics identified from the input data could inform which of those base SSMs is to serve as a starting point for generating the patient-specific 3D model. A population could be defined at least in part by a certain affliction, like hallux valgus or metatarsus adductus, exhibited, but does not need to be related to any affliction per se; it could be defined by any other characteristic, trait, or the like.

Continuing with FIG. 7, the process assigns the planes (760 of FIG. 7), i.e., decides which one of the submitted views of the input data corresponds to which plane of the 3D representation (distal planar view, lateral view, etc.), normalizes the plane orientations to align with a pre-defined coordinate system, scales the structure models, and performs output validation. For scaling, any one or more options are possible. Examples include scaling based on input (from 110) indicative of patient anatomical size (such shoe size, foot length, etc. in the case of a foot), using image metadata to reconstruct scale, introducing objects of known dimensions in the scan/imaging process, i.e., use scale markers, and/or asking the user to input scale-related information and directly relate that to the images, as examples.

With respect to the output validation, it may be known how the planes being viewed should sit relative to the anatomy and one another. There may be criteria used for this, for instance. The validation effort can therefore identify any scanning errors in this way. The strictness of the validation can be set to any level desired, and can be adjusted depending on the anatomy, type of scans involved, and/or other factors. The content of the scans (the anatomy shown) and the resulting model can also be normalized such that—in the example of a foot-a ground plane/floor may be aligned with the long side of the lateral scan (the length of the foot). Additionally or alternatively, the process collects a distal first metatarsal rotation against a virtual floor plane acquired indirectly from the estimate, which would be the plane with normal parallel to the shorter (vertical) side of the lateral view. When measurements are taken, they can be done relative to the floor plane (in this example), which serves as a ‘horizontal’ reference. In the context of a foot, the process can consider both dorsoplantar and lateral views regardless of whether it is a left or right foot (for the dorsoplantar view, the view is about 15 degrees ‘forward’).

The process can additionally run axis fitting/refinement and measurement steps (762 of FIG. 7) as might be performed in workflow 201a, and export (764 of FIG. 7) the data as 3D output 212 for use by a surgical planning module performing surgical planning and procedures 214.

The generating (120) of the patient-specific model can include a segmentation step that is performed automatically and/or manually. In some embodiments, there may be manual pre-segmentation options adjustments, though in other examples the segmentation is fully automated. The automatic segmentation can include running image data through a neural network to perform a ‘crude segmentation’, e.g., identification of which structure is which, landmarks on the anatomy, etc., as examples. Then, processing assembles a patient-specific model of the anatomical region of the patient based on the AI model work from the anatomical context 208 and a knowledge of what a ‘normal’ patient anatomical region looks like.

In terms of validation for surgical planning, a goal may be to have a singular view in the report, the singular view containing a projective view and a few primary axes used for analysis/correction to manipulate structures in the anatomical region. A user can use these to partially validate the used input data.

The 3D model may be generated within, and/or assigned, a coordinate system. Further, the model may be annotated. Annotations were discussed as part of 3D analysis 204 and anatomical context processing 208, where a neural network creates rough 2D or 3D segmentations (labels) of the input image(s). In the 3D context (204), the processing can segment the 3D structural (e.g., bone) models from the 3D images, and in the 2D context (208) the processing can reconstruct 3D structural models from a plurality of 2D images.

In examples, as part of the 3D output 212, multiple axes of anatomical structures are fit on the 3D model(s) and various properties are measured and indicated as preparation for the surgical planning module 214.

In examples, distance mapping is used to examine, and if desired display to a user, how far anatomical components in the 3D model are from other components in the 3D model. This can be useful when showing a simulation of a correction or when taking needed measurements, for instance. With the 3D anatomical model, distance mapping samples the distance from a surface point, such as a bone surface point, in the 3D model along a surface normal to an encountered anatomical component. The surface normal refers to a direction perpendicular to the surface on which the surface point sits. This sampling can be done for each of several surface points in the 3D model. For each sample, the distance can be measured starting at the surface point and tracking/traversing along the surface normal away from the surface until another anatomical component, for instance another bone, in the model is encountered. This gives a distance between the surface point (e.g., of the bone) and an adjacent component (e.g., bone), for instance.

This information can be analyzed for each of several surface points and then represented graphically, for instance as a color map. The process can notify a user of the software if the 3D model reflects a problematic anatomical arrangement, for instance overlapping bones or anything else that can be taken as an anatomical impossibility and therefore indicate a possible error in the segmentation processing. In this regard, the distance mapping provides a ‘sanity check’ for the 3D model.

This distance mapping and color coding based on distances could be used on a 3D model of a current (pre-operative) patient anatomy to inform of abnormalities in a joint, positioning of an anatomical component in the joint relative to another anatomical component, and so on. This can be useful to a physician or other user about abnormalities beyond what the user might identify from just looking at the model without any distance-based color coding applied to it. Additionally or alternatively, the distance mapping and color coding could be used on a proposed post-operative 3D model to assess post-operative patient anatomy, for instance to check or verify whether post-operative position as reflected by the post-operative model is back to a ‘normal’ and/or a patient ‘original’ range (i.e., prior to development of the condition). In this regard, the distance mapping against a post-operative 3D model can be used to check (automatically and/or by a user) whether a surgical procedure has accomplished a desired adjustment, for instance accomplished a normative corrective position. In the case of a bunion correction, it can inform whether the distance(s) between selected anatomical features of the foot are within desired range(s), for example.

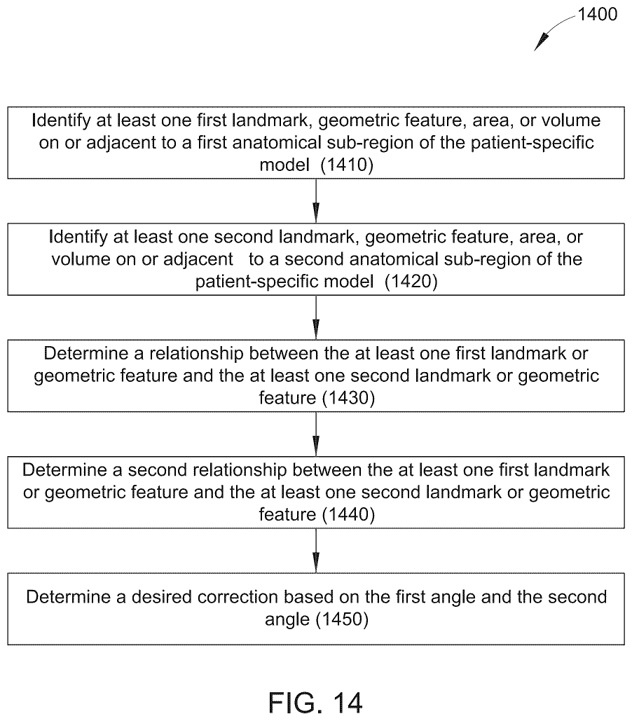

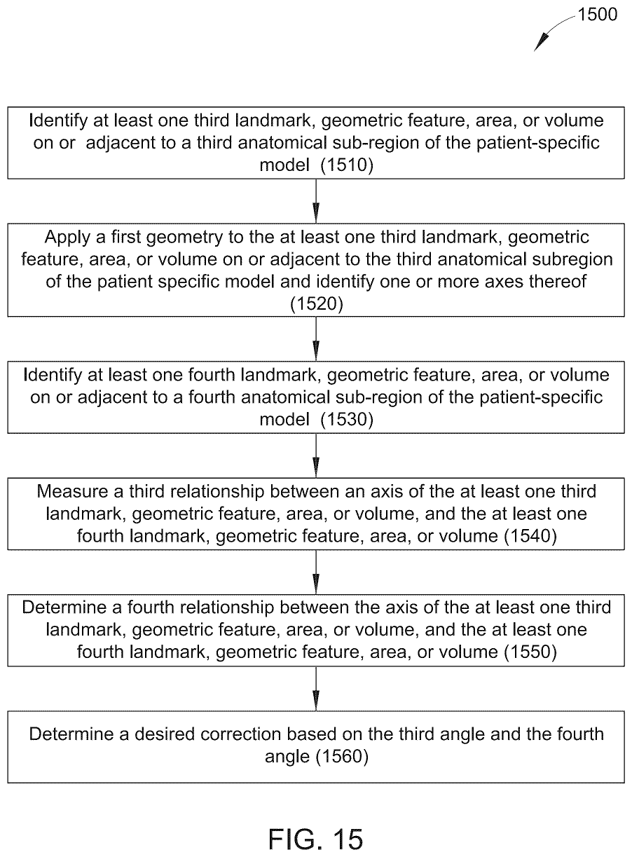

Continuing with the process of FIG. 1, after generating the patient-specific model for the anatomical region the process identifies (130), within the model, one or more anatomic landmarks within the anatomical region of the patient. Beyond surfaces and contours of anatomical structures, example landmarks are axes, for instance bone axes. In the context of a Lapidus procedure discussed herein, two main deformities may be measured, identified, and corrected, albeit at least partially concurrently in software as described below with reference to FIGS. 14 and 15. For instance, a bunion is anatomically corrected by correcting the angle between the long axis of the first and second metatarsals (intermetatarsal angle-“IMA”), and de-rotating the first metatarsal. Correcting the deformities of a bunion is not as straight-forward because rotating the first metatarsal to address the rotational deformity in many cases also lessens the IMA (but in some cases, perhaps not enough to lessen the angle to the desired angle to correct the deformity).

Continuing with FIG. 1, the process collects (140) one or more measurements within the anatomical region based on the landmarks, and calculates (150) a difference between the collected measurements and a set of normal values for the measurements. Then, based on the calculated differences, the process identifies (160) deformities in the anatomical region. Based on this, the process identifies (170) corrected position(s) of one or more anatomical structures and identifies (180) one or more corrections that, when performed, manipulate the one or more anatomical structures within the anatomical region from respective deformed position(s) to the corrected position(s). With respect to this aspect 180, these corrections might be a composite of manipulations that result in target positions. As alluded to above with reference to correction of bunion deformities, the manipulations might not be independent of each other, meaning that performance of one manipulation, such as an angular adjustment of the angle between two bones, might cause another manipulation, such as a rotational change in one of those bones. Thus, it might be that a net angular change of 5 degrees is desired between two bones and a net −3 degree rotational change is desired in one of those bones but that effecting this in two manipulations—a 5 degree angular change followed by a −3 degree rotation of the one bone-results in a net angular change and/or net rotational change that is other than the 5 degrees/−3 degrees desired. The 5 degree angular manipulation might in itself have imparted a −1.5 degree rotation, for example, in which case a further −3 degree rotation results, undesirably, in a net −4.5 degree rotational change.

The deformity can therefore be corrected with a more holistic, or composited, approach being taken to determine the proper corrections. Conventionally when addressing a bunion, the angular and rotational corrections to make would not be taken as a composite determination. In accordance with aspects described herein, the corrections can be determined holistically in order to, for example, generate accurate hardware such as cut guides, implants, etc.

Based on the measured deformed positions and calculated corrected positions, the process of FIG. 1 continues by generating (190) one or more hardware components. Part of hardware generation may be generation of specifications of such hardware, for instance specifications for 3D-printable hardware, the specifications being data/files to guide the printing or other formation of hardware by hardware generating equipment. The hardware components may be, for instance, guides for surgical manipulation, cutting, correction, or other procedures. For instance, the hardware might include cut guides that precisely control cuts to be made to anatomical features, correction guides for anatomical corrections, fixation devices for osteofixation, and/or implants, as examples.

Aspects of FIGS. 1-7 can be performed or facilitated by computer systems usable by end users, for instance medical personnel. The computer systems can execute software to perform aspects described herein and facilitate interaction with users, for instance input and output. In examples, a front-end is provided as user-facing software. The front-end could be a web application, for instance, with analyzed cases and downloadable reports for the cases. The downloadable reports can be reports of surgical planning and procedures, including presentation of any data, determinations, or the like discussed herein.

The front-end can be contrasted to a back-end of the software, which can run on one or more computer systems that may or may not be or include computer system(s) running the front-end. In examples, backend components include components implementing aspects of FIGS. 1 and 2, for example database(s), analyzers, and processers of image data (x-ray, CT, etc.), surgical planning and procedures module, and a reporting component. In a particular example, backend components run in a public, private, or hybrid cloud environment with application programming interfaces and management thereof. One or more databases may be provided, for instance to hold biometric and/or other supplemental information regarding the patients, for instance demographic, height/weight and other body properties, diagnoses, performed operations, etc. An analysis database may also be provided. In some examples, the database information can be combined for results from actual image analysis and possibly to detect/learn recurring patterns from this big data. The cloud platform can run container(s) and/or other processing environments (virtual machines, etc.) to implement components and perform functions such as for receiving image data input, performing analysis and other activities described herein against 3D and 2D images, implementing a surgical planning and procedures module, and generating reports and images.

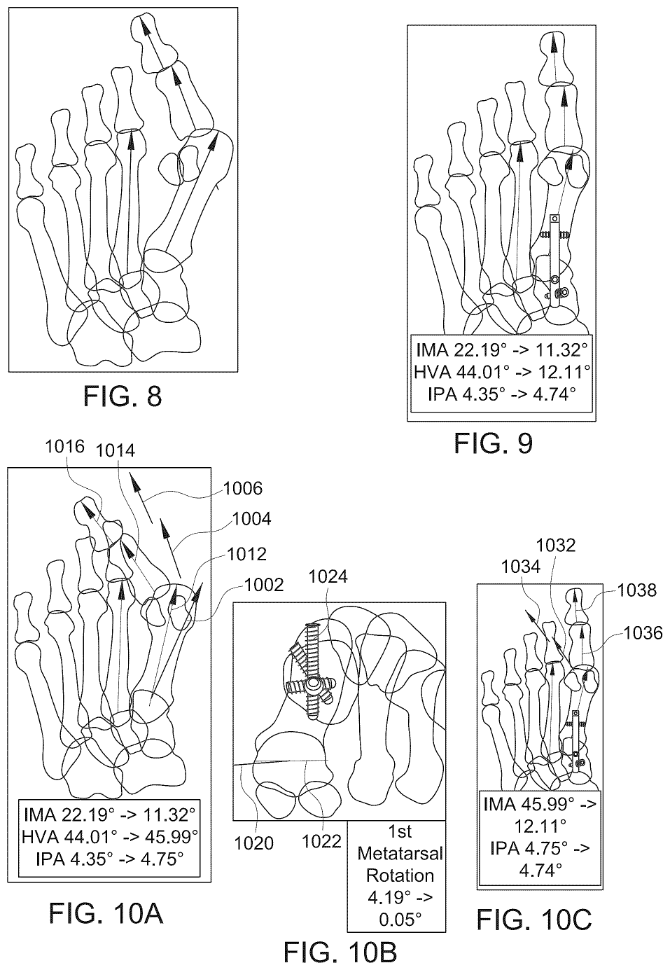

Disclosed aspects will be further illustrated and described with reference by way of example to a Lapidus procedure. FIGS. 8 and 9 depict example representations of patient anatomy before and after a Lapidus procedure. Initially, FIG. 8 depicts a pre-operative patient anatomy showing positions of bones indicative of a deformity referred to as a bunion (“hallux valgus”). The lines imposed on the first and second metatarsals, as well as the first proximal and distal phalanges (i.e., of the big toe) show axes of the respective bones. Example pre-operative measurements are:

| Measurement | Value (degrees) |
|---|---|
| 1st-2nd Intermetatarsal Angle (IMA) | 22.19° |
| Interphalangeal Angle (IPA) | 4.35° |
| 1st metatarsal Rotation | 4.19° |
| Hallux Valgus Angle (HVA) | 44.01° |

A positive 1^{st} metatarsal rotation indicates pronation, while negative indicates supination.

FIG. 9 depicts an example post-operative (corrected) patient anatomy, again with lines on the first and second metatarsals, as well as the first proximal and distal phalanges (i.e., of the big toe) showing axes of the respective bones. A fixation device is also reflected. Example post-operative measurements are:

| | After correction | Amount of total |
|---|---|---|
| Measurement | (degrees) | correction (degrees) |
| 1st-2nd Intermetatarsal Angle | 11.32° | −10.87° |
| (IMA) | | |
| Interphalangeal Angle (IPA) | 4.74° | 0.39° |
| 1st metatarsal Rotation | 0.05° | −4.14° |
| Hallux Valgus Angle (HVA) | 12.11° | −31.90° |

A positive amount of correction indicates an increase in measurement value, while a negative indicates a decrease.

The anatomical corrections involved in a Lapidus procedure can be generalized to an intermetatarsal angle correction, a first metatarsal rotation angle correction, and a hallux correction (which may not be a separate step). Further details are provided with reference to FIGS. 10 through 16.

FIG. 10A depicts an example intermetatarsal angle (IMA) correction. The correction reflects an angular adjustment (e.g., decrease) between the first and second metatarsals of the foot if considered in insolation of any other corrections (for instance a rotation) that might be performed as part of a standard Lapidus procedure. Lines 1002, 1004, 1006 represent a position, pre-IMA correction, of the first metatarsal, proximal phalange, and distal phalange, respectively. Lines 1012, 1014, 1016 represent position, post-IMA correction, of the of the first metatarsal, proximal phalange, and distal phalange, respectively.

FIG. 10B depicts an example first metatarsal rotation correction with lines 1020 and 1022 representing a rotation imparted on the first metatarsal. Line 1020 represents an axis pre-rotation and line 1022 represents an axis post-rotation. Also shown are fixation device(s) 1024.

FIG. 10C depicts a Hallux correction with lines 1032 and 1034 representing pre-correction positions of the first proximal and distal phalanges, respectively, and lines 1036 and 1038 representing post-correction positions of the first proximal and distal phalanges, respectively. The Hallux correction rotates the first proximal and distal phalange around the metatarsophalangeal (MTP) joint so that Hallux valgus angle is brought to a desired/normative reference value. As noted, the Hallux correction of FIG. 10C might not be implemented as a separate correction/procedure, as it might be imparted as part of the IMA and rotational corrections.

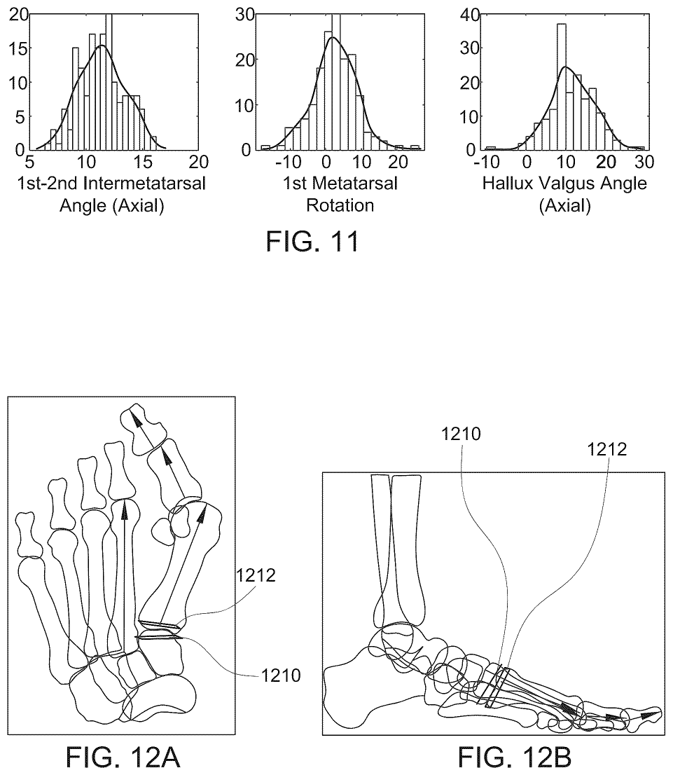

A surgical planning and procedures module (e.g., 214, FIG. 2) might provide, in the context of a Lapidus procedure, an extensible framework for performing a series of operations. Normative reference values of all desired measurements may be determined from set(s) of images, for instance computed tomography images. FIG. 11 depicts example normative measures of interest for Lapidus and Hallux correction procedures.

In FIG. 11, three distribution graphs are presented for: (i) intermetatarsal (first-second) angle correction, in degrees on the x-axis, when considering an axial (top-down) view, (ii) first metatarsal rotation, in degrees on the x-axis, and (iii) Hallux Valgus angle (proximal to distal), in degrees on the x-axis, when considering an axial (top-down) view.

The Lapidus and Hallux correction procedures aim to return selected measures of interest to a normative reference value, or at least a value appropriate for the patient under the circumstances.

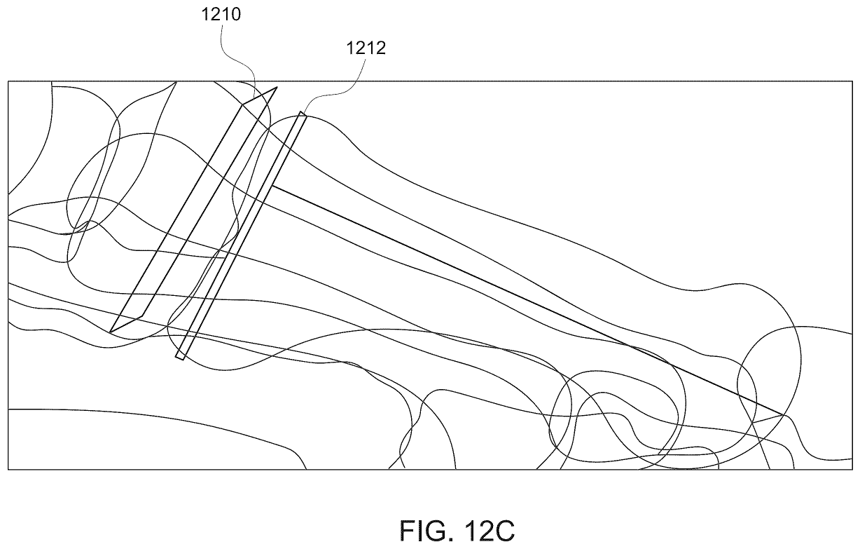

Lapidus arthrodesis refers to a procedure to cut and fuse two bones of the foot—the first metatarsal and the medial cuneiform. FIGS. 12A-12C depict example osteotomy locations for a Lapidus arthrodesis as represented on top (FIG. 12A) and sagittal (FIG. 12B) plane views of a patient foot. FIG. 12C depicts a closer view of an area depicted in FIG. 12B. In order to correct a bunion, a Lapidus arthrodesis can be used to provide a multiplanar correction incorporating IMA adjustment and de-rotation. But, as noted previously when discussing corrected positions of anatomical structures and interdependencies existing between corrections of different structures, manipulations of structures might not be independent of each other. Here, movement of the distal portion of the first metatarsal in the lateral direction can shrink the IMA. In examples, a manipulation is performed as a pivot or modified pivot about a point on or near the head (proximal end) of the first metatarsal, for example. With the lateral movement/pivot, the first metatarsal may be caused to rotate about its long axis. As viewed looking along the long axis of the metatarsal from proximal portion to distal portion thereof, the first metatarsal rotates about the long axis in a counter-clockwise direction (if the left first metatarsal) and a clockwise direction (if the right first metatarsal). Similarly, a rotation of the first metatarsal might inherently decrease the IMA. Consequently, the desired IMA and rotational amounts are to be composited into an overall correction, which itself might be implemented in one or more steps. In an example, the overall correction is implemented by precise osteotomy locations for the Lapidus arthrodesis and fusion. The osteotomy locations here are cut planes and their locations and other properties (including the angles of the cut planes relative to reference of given planes such as one perpendicular to the ground) are significant. One such plane (1210) is located on the distal end of the medial cuneiform and the other (1212) is located on the proximal end of the first metatarsal. These two bones are to sit flush with each other, and a fixation is applied to fuse them together.

In performance of the arthrodesis, a first metatarsal saw may be aligned with the joint gap and a medial cuneiform saw may be placed so that when the cut planes are aligned and rotation is corrected, the IMA and rotation are brought to (or within some threshold of) a desired reference value, for instance a normative value for the patient. Both planes may be moved along their plane normals so that a whole articular region is cut.

One approach for estimating rotation is to measure a first metatarsal (MT1) rotation angle from the MT1 distal head articular surface, further details of which are discussed below with reference to FIG. 15.

The Lapidus procedure can include a phantom intramedullary nail placement procedure and nail selection. A nail and attachment screws, and placement, are shown in FIG. 10C to fuse the first metatarsal to the cuneiform.

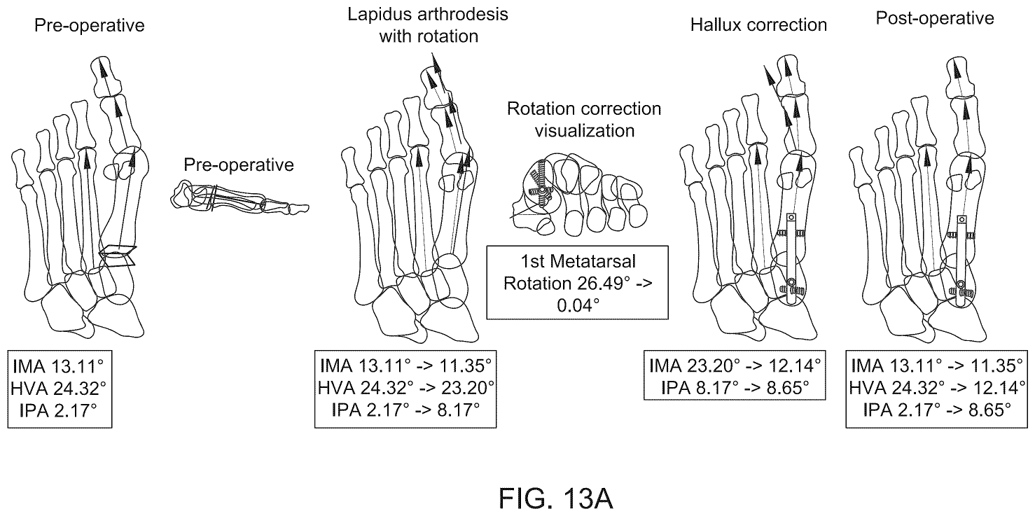

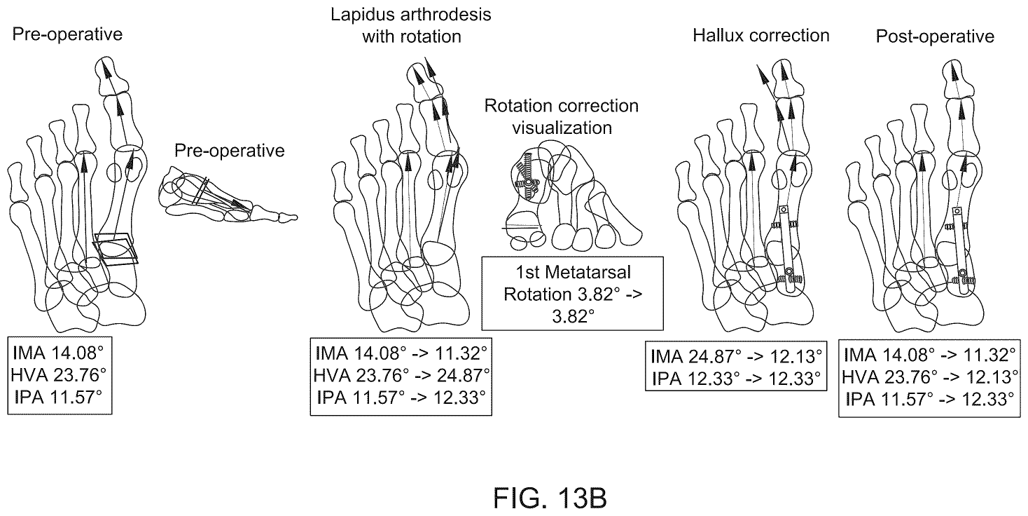

FIGS. 13A and 13B depict example visualizations of pre-correction and post-correction anatomy in Lapidus procedure planning, and optionally intra-operative steps, for example showing IMA correction before and after rotation correction, which may be provided by software executing to perform aspects described herein. In examples, a visual simulation can be generated automatically and presented on a display device for a user to visualize the transition of anatomical structure(s) made as part of the corrections. The visual simulation can use the 3D model and the representations of the structures as provided by the 3D model to show the transition of structure(s) from a first position to one or more other positions. One such other position, a second position, may be a target position that is consistent with target anatomical value(s) for the patient's anatomical features exhibiting the deformity/deformities. In some examples, the visual simulation can show a progression of the structure(s) through additional position(s) as the structure(s) are transitions from the first position to the second position. In this manner, the visual simulation can be an animation showing movement(s) being effected by the correction(s) being made. The correction(s) may be effected at least one surgical activity, for instance cut(s), coupling/decoupling of instruments, and so on, and the visual simulation can further include simulation(s) of such surgical activity, showing the activity relative to the anatomical structure(s), as represented in the 3D model, to effect the correction(s) being made.

The generated 3D model or portions thereof can be conveyed to a physician, patient, or other user visually on a display device as discussed above and/or provided in the form of 3D printed models. Examples of such models/portions include models of the first metatarsal and the medial cuneiform separate from each other, models of the first metatarsal and the medial cuneiform in an integral configuration (in one or both of deformed and proposed corrected positions), and/or models of the entire patient foot with the first metatarsal and medial cuneiform (in one or both of deformed and proposed corrected positions), as examples.

It is noted that in connection with a Lapidus correction discussed above, an additional correction might be needed relative to the patient's big toe, for instance a further straightening (via Akin osteotomy for instance to correct angle of big toe) in some situations, and if desired.

FIG. 14 depicts an example process 1400 for anatomic correction, in accordance with aspects described herein. The process of FIG. 14 can represent an example of aspects of FIG. 1 tailored to the Lapidus procedure case discussed previously, in which case the process of FIG. 14 in the context of a Lapidus procedure can provide intermetatarsal angle correction. The process includes identifying (1410) at least one first landmark, geometric feature, area, or volume on or adjacent to a first anatomical sub-region of a patient-specific model (e.g., 3D model). An example first landmark, geometric feature, area, or volume in the context of a Lapidus procedure is the long axis of the first metatarsal. An example first anatomical sub-region of the patient-specific model in the context of a Lapidus procedure is the first metatarsal (or medial column). The process also identifies (1420) at least one second landmark, geometric feature, area, or volume on or adjacent to a second anatomical sub-region of the patient-specific model. An example second landmark, geometric feature, area, or volume in the context of a Lapidus procedure is the long axis of the second metatarsal, and an example second anatomical sub-region of the patient-specific model in the context of a Lapidus procedure is the second metatarsal. 1410 and 1420 can correspond to 130 of FIG. 1.

The process determines (1430) a relationship (“first relationship”) between the at least one first landmark or geometric feature and the at least one second landmark or geometric feature. In the context of a Lapidus procedure, this determination can be a measurement of an angle (“first angle”) formed between the at least one first landmark or geometric feature (long axis of first metatarsal) and the at least one second landmark or geometric feature (long axis of second metatarsal). This first angle can be a measurement of an initial angle, the initial angle being the IMA associated with the anatomy in the deformed (e.g., bunion) state.

The process also determines (1440) a relationship (“second relationship”) between the at least one first landmark or geometric feature and the at least one second landmark or geometric feature. In the context of a Lapidus procedure, this determination can be a desired angle (“second angle”) between the at least one first landmark or geometric feature (long axis of first metatarsal) and the at least one second landmark or geometric feature (long axis of second metatarsal). This second angle can be a desired corrected angle, i.e., a desired IMA that the anatomy should reflect in a post-operative state to correct the deformity (e.g., bunion). For instance, the desired angle might be 11 degrees if the desire is to correct the patient to a 11 degree IMA. The desired angle, or more generally the desired relationship, can be determined in any desired manner. In one example, it is an average or other reference value based on a population (such as a “normal” value, or a range that is considered normal). Alternatively, it could be calculated and/or recommended by software based on the anatomy of the patient, or input manually by a user. In the context of a Lapidus procedure, the desired relationship could be a desired angle that is determined/selected as or based on (i) a known normal angle, (ii) a desired angle calculated and recommended by software based on the anatomy of the patient, and/or (iii) a desired angle provided by a physician, which could be input directly and/or a recommended or normal value that is tweaked by the physical.

The process of FIG. 14 additionally includes determining (1450) a desired correction based on the first and second relationship. In the context of a Lapidus procedure, the first and second relationships could be the first and second angles discussed above, and therefore a correction is determined based on these angles. The correction could therefore encompass an IMA correction to impart by way of a surgical procedure, for instance a Lapidus arthrodesis. In this regard, and as explained previously, the IMA and rotational corrections of a Lapidus procedure may be interdependent. Because of this, simply determining the corrective action (1450) to be an angular adjustment equal to the difference between the first and second angles may not be appropriate, since a needed rotational correction might impact the IMA. Consequently, the determination of the desired correction (1450) may be determined in conjunction with processing of FIG. 15, depicting an example process for a rotational correction, for instance in connection with a Lapidus procedure.

FIG. 15 involves application of a geometry to a landmark to aid in correction determination. In the context of a Lapidus procedure, the correction is a rotational aspect and the geometry is a cylinder, and more specifically what is referred to geometrically as a truncated prolate spheroid with circles of equal size on top and on bottom, i.e., a “barrel” shape. The landmark in this case is the distal-most portion of the first metatarsal. A cross section of this portion may be viewed against the 3D model, and the barrel can be fit thereto. The central axis of the geometry can then be used in comparison relative to some reference (such as a true horizontal/ground plane or some other reference plane) to account for a rotational deformity.

Referring to FIG. 15, the process identifies (1510) at least one third landmark, geometric feature, area, or volume on or adjacent to a third anatomical sub-region of the patient-specific model. In the context of a Lapidus procedure, an example third landmark, geometric feature, area, or volume may be the distal-most portion of first metatarsal, and the third anatomical sub-region of the patient-specific model may be the first metatarsal.

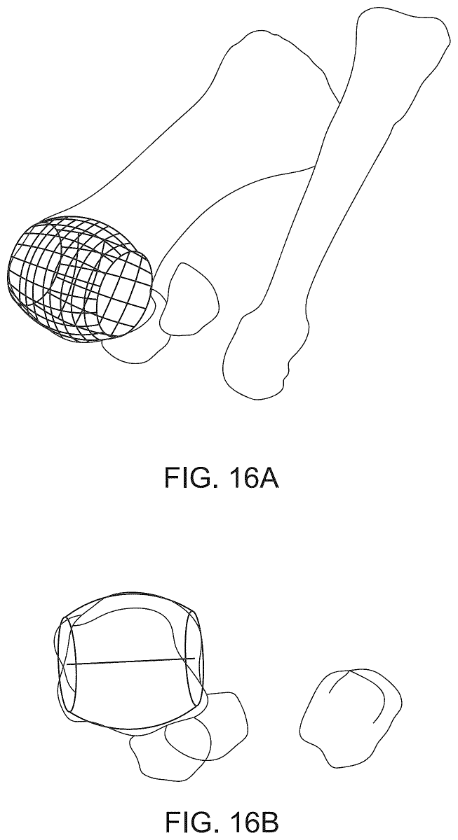

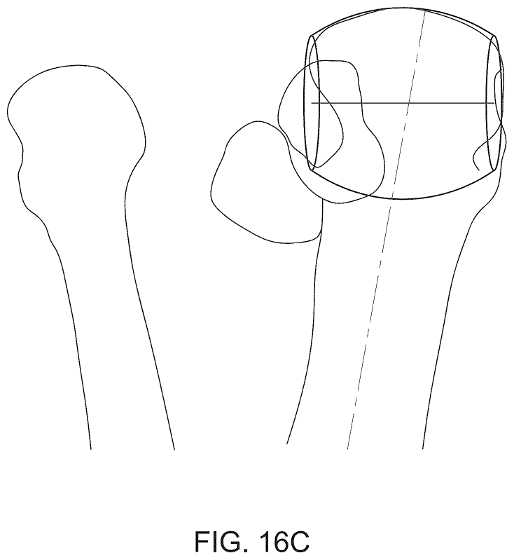

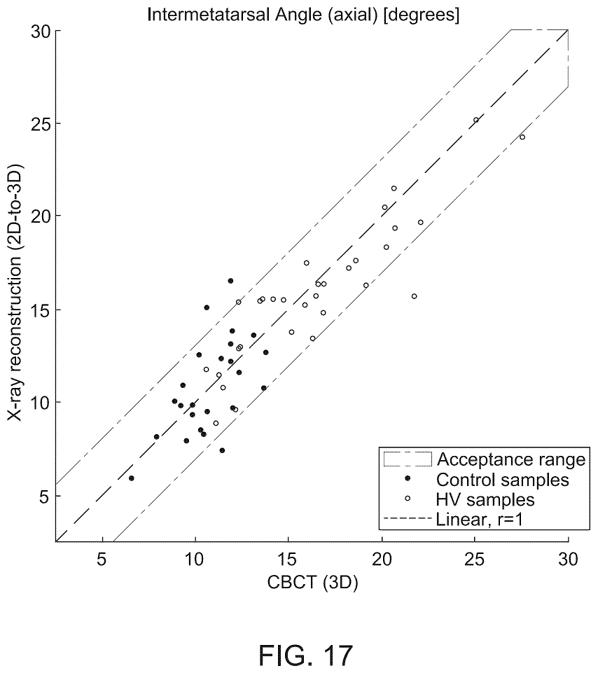

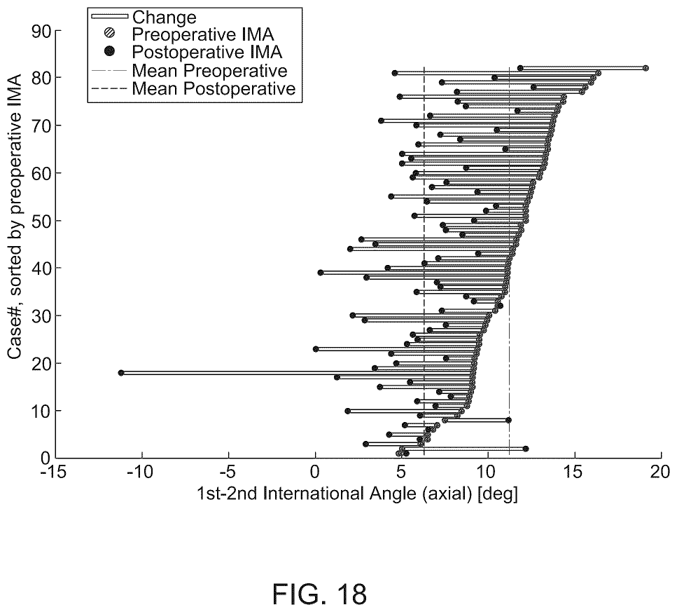

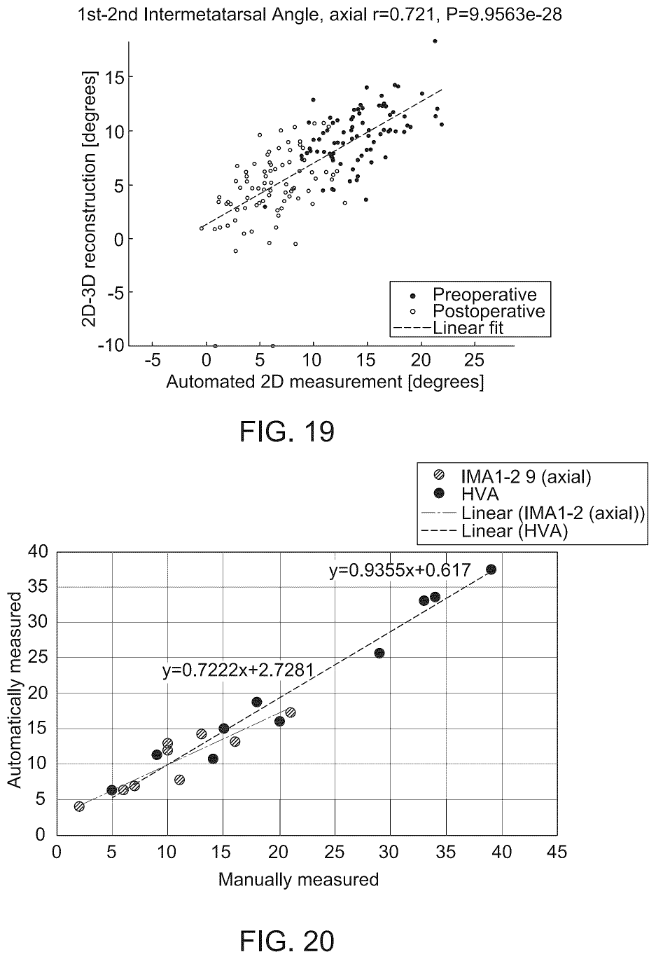

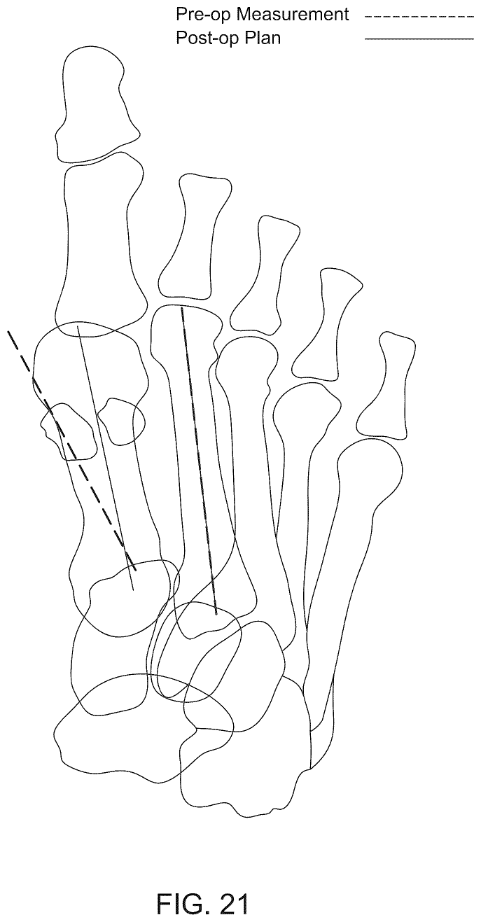

The process then applies (1520) a first geometry to the at least one third landmark, geometric feature, area, or volume on or adjacent to the third anatomical subregion of the patient-specific model and identifies one or more axes thereof. In the context of a Lapidus procedure, the geometry may be a barrel/modulated cylinder three-dimensional geometry, and the one or more axes can include a central axis of the barrel. In this application, or fitting, of the geometry, the geometry may be fitted to the third anatomical subregion/anatomy may be a bone surface, for instance the distal articular surface of the 1st metatarsal. The barrel axis can serve as an estimate of the first metatarsal's distal mediolateral axis, which may then be used, together with a fourth landmark (such as a floor plane as explained below with reference to 1530), to measure a correction (such as a metatarsal rotation around its longitudinal axis). FIGS. 16A-16C provide example depictions of a barrel geometry applied to the distal-most portion of first metatarsal. FIG. 16A depicts a superposition of the barrel geometry and a 3D triangle-mesh representation of the first metatarsal bone. FIG. 16B and FIG. 16C show outline of the barrel geometry on top of outlines of the 3D triangle-meshes in a plane perpendicular to the first metatarsal's longitudinal axis, and in an axial plane, respectively.

Continuing with FIG. 15, the process identifies (1530) at least one fourth landmark, geometric feature, area, or volume on or adjacent to a fourth anatomical sub-region of the patient-specific model. In the context of a Lapidus procedure, a fourth landmark, geometric feature, area, or volume may be an area beneath the foot, and the fourth anatomical sub-region of the patient-specific model may be a ground plane or horizontal against which rotational deformity may be measured. Depending on how the image data (e.g., x-ray) was captured, such ground plane might need to be defined. The process could use an axial plane provided/defined by the imaging device used to capture the initial image and/or an axial plane provided/defined by the patient-specific model, for instance.

The process measures (1540) a third relationship between an axis of the geometry fit to the at least one third landmark, geometric feature, area, or volume and the at least one fourth landmark, geometric feature, area, or volume. In the context of a Lapidus procedure, the third relationship can be an angle (“third angle”) between the axis of the geometry that was fit at least one third landmark and the area beneath the foot, and the relationship is an angle that is the measurement of the initial rotational deformity. In other words, in the context of the Lapidus procedure, the rotational deformity of the first metatarsal is measured from the first metatarsal distal mediolateral axis (e.g., as informed by the barrel axis) and the floor plane, in a direction perpendicular to the first metatarsal longitudinal axis.

With the third relationship measured, the process then determines (1550) a fourth relationship between the axis of the geometry that was fit to the at least one third landmark, geometric feature, area, or volume, and the at least one fourth landmark, geometric feature, area, or volume. In the context of a Lapidus procedure, the fourth relationship can be a desired angle (“fourth angle”) between the axis of the geometry that was fit to the at least one third landmark and the area beneath the foot, i.e., the desired angle for the corrected (rotationally) position. This fourth angle can be determined/selected as or based on: (i) a known “normal” angle, (ii) a desired angle calculated and recommended by software based on the anatomy of the patient, and/or (iii) a desired angle provided by a physician, as examples.

The process of FIG. 15 then continues by determining (1560) a desired correction based on the third relationship and the fourth relationship, for example the third and fourth angles in the context of a Lapidus procedure. The correction could therefore encompass a rotational correction to impart by way of a surgical procedure. In this regard, and as explained previously, the IMA and rotational corrections of a Lapidus procedure may be interdependent. Simply determining the corrective action (1560) to be a rotational adjustment equal to the difference between the third and fourth angles may not be appropriate, since a needed IMA correction might itself provide some rotation. Consequently, the determination of the desired correction (1560) may be determined in conjunction with processing of FIG. 14.

Therefore, some aspects of FIGS. 14 and 15 may be performed before, concurrent with, or after each other, though both might involve the determination of a correction (e.g., at 1450 of FIG. 14 and 1560 of FIG. 15), which may be implemented in software as a same determination, for instance a composite determination based on the processing that occurs in FIGS. 14 and 15. Alternatively, they could be separate determinations that account for each other—for example in FIG. 14 if the difference between the first and second angle is 11 degrees, this could be determined as part of 1450 which then adjusts that based on a rotational correction determined according to FIG. 15 (i.e. as part of 1560), to produce a desired correction that is actually −10.5 degrees, as an example. Similarly, a rotational amount could be determined as part of FIG. 15 (1560) but then adjusted as part of 1560 based on the angular correction (from FIG. 14) that is desired. As yet another example, each determination (1450, 1560) could be performed without regard to the other (e.g., determine apparent corrections as an apparent angular adjustment for IMA (say −11 degree IMA correction based on an apparent 11 degree angular correction needed) and apparent rotation (say a −4.2 degree rotation based on an apparent 4.2 degree difference), which are then each input into another determination (not depicted) that determines a composite/actual adjustment based on these apparent corrections, the composite being, for instance, an actual IMA adjustment of −10.5 degrees together with a −3.8 degree rotation to effect the desired post-operative anatomical outcome desired.

It is noted that while normative reference values were used in the example algorithm above for IMA correction and bone rotation, aspects discussed herein enable the creation of a solution that is specific to each patient. For example, a patient may be encountered who has a deformity in terms of IMA (e.g., IMA greater than the reference value) but no rotational deformity. In such a case, it would be desired to decrease and correct the IMA, but it would also be desired to identify that the patient does not have a rotational deformity, and for the operative planning to indicate that there is no need for rotational correction (or vice-versa for a rotational deformity with no IMA deformity). Otherwise, a risk exists of de-rotating the metatarsal when no rotational deformity exists, thus creating a new problem for the patient. In identifying whether a deformity exists, for IMA or rotation, a process could determine whether the patient's IMA or rotation value (in these example) is some threshold amount different from a ‘normal’ value. For example, if the ‘normal’ value for IMA is taken to be 11 degrees, the process could classify an IMA deformity in a patient if that patient's IMA is measured to be more than 3 degrees different from that normal value. In this regard, and as more data is collected, deformities may be classified in terms of as how many standard deviations from the ‘normal’ value a patient's IMA is. It is emphasized that the foregoing points are made with reference to IMA but could also be applied for rotational deformity. And even more broadly, different procedures will necessitate that different measurements of different anatomy be considered, and such measurements would have their own applicable normal/reference values to consider.

Example features described herein include a method directed to generation of a 3D model from 2D imaging data. The method can include obtaining 2D imaging data of an anatomical region of a patient. The anatomical region can include patient anatomical features. Anatomical features can be anything that can be compared against determined ‘normal’ or other reference features, such as features of a general or specific population of other individuals. They may be, or at least relate to, properties (dimensions, shape, orientation and position, etc.) of physical anatomical structures, and are presented and ascertained via the anatomical structures present in the anatomical region of interest. The method can also generate, using the 2D imaging data, a 3D model of the anatomical region of the patient. The 3D model is specific to the patient and provides a 3D representation of the patient anatomical features. One or more deformities of the anatomical features can be exhibited in the 3D model (though they may or may not be visually discernible to a user viewing the 3D model on a display), the deformities referring to deviations from the reference features. Example deformities could be anatomic components that deviate from a traditional or reference position, for instance due to trauma, for example fractures. Another example of deformities could be anatomic components that lack traditional features/characteristics (including, for example, a bone with an abnormal curvature, etc.).

The method can proceed by identifying such one or more deformities of the patient anatomical features. The deformities are identified based on the 3D model and are determined relative to target anatomical values for the patient anatomical features (e.g., ‘normals’ or other reference values). The identification of the deformities could include, for instance, identifying anatomical landmarks of the patient (e.g., angles, distances, orientations, or any other measurements to characterize patient anatomy), as exhibited in the 3D model, taking/obtaining anatomical measurements based on those anatomical landmarks, then comparing the anatomical measurements to the target anatomical values, and determining the one or more deformities based on that comparing. The target anatomical values could be or include definite values for anatomical measurements and/or desired ranges into which the anatomical measurements are to fall.

With the one or more deformities determined, the method can then determine, based on a relationship between the anatomical measurements and the target anatomical values, at least one correction to make to at least one anatomical structure of the patient. An example such relationship is a difference between patient anatomical measurement(s) and reference value(s). The difference might inform correction(s) of, or based on, that amount. A correction could indicate at least one corrected position for an anatomical structure or collection of anatomical structures. The corrections made to anatomical structures are ultimately to address the deformities by providing corrections that, when made, produce updated anatomical measurements. In some cases, a correction need not be one that conforms the patient anatomy to the reference values but instead brings the values associated with the patient anatomy more in accordance with, e.g., closer to, the reference values (to fall within desired range(s), for instance).

In situations involving patient-specific hardware to facilitate the at least one correction to make to the at least one anatomical structure, a method can optionally generate a specification of the hardware. The specification can be generated based on (i) the representation of the patient anatomical features as provided by the 3D model and (ii) the determined at least one correction(s). The specification can include measurements/parameters tailored to the patient based on the representation of the patient anatomical features as provided by the 3D model and on the determined correction(s). In examples, the patient-specific hardware includes at least one hardware guide for guiding surgical procedure(s) to provide the correction(s) to make to the at least one anatomical structure. The hardware guide(s) can include, for instance, cut-guide(s) for cutting procedure(s).

Optionally, a method can generate a visual simulation, where the visual simulation graphically presents a transition of the patient anatomical structure(s), as represented in the 3D model, from a first position to a second position. The first position can be an existing, pre-operative position, and the second position can be a position that is consistent with the target anatomical values for the patient anatomical features. The correction(s) may be effected by surgical activity/activities (cut(s), coupling/decoupling of instruments, etc.), and the visual simulation can include simulation(s) of the surgical activity/activities relative to the patient anatomical structure(s) as represented in the 3D model.

Additionally or alternatively, a method for generating a 3D model can include obtaining 2D imaging data of an anatomical region of a patient, where the anatomical region includes patient anatomical features, and generating, using the 2D imaging data, a 3D model of the anatomical region of the patient, the 3D model being specific to the patient and providing a 3D representation of the patient anatomical features. The generation of the 3D model can include performing anatomical context processing that includes annotating, using an artificial-intelligence (AI) model for instance, contours/edges/surfaces of anatomical structures, of the anatomical region, presented in the 2D imaging data, and performing 2D-to-3D reconstruction that optimizes orientation, placement, and scale of 3D digital volumes modeling the anatomical structures (e.g., the digital volumes representing anatomical structures such as bones for instance) to yield, as the 3D model, a 3D anatomical representation of the anatomical region. In embodiments, the 2D imaging data includes two or more images/different views of the anatomical region. The images/different views can be radiographs, for instance.

An AI model, if utilized, can perform image segmentation in two dimensions to identify locations for the contours/edges/surfaces of the anatomical structures. The image segmentation can produce 2D segmentations. The method can process the 2D segmentations into a set of 2D points, and this can be based on determining, for each anatomical structure of the anatomical structures, landmarks of the anatomical structure, the landmarks of the anatomical structure including, for instance, a centroid of the anatomical structure.

The annotating of features of the anatomical structures can provide annotations that include outlines of peripheries of the anatomical structures. In examples, the anatomical structures include bones, and the annotations show the bones contoured on one or more 2D image planes. The method could display, on a display, the outlines of the peripheries of the anatomical structures, with the outlines being presented with varying graphical properties, such as different colors for different bones, to facilitate identification and distinction between the anatomical structures.

In examples, the 2D-to-3D reconstruction includes fitting together, in 3D space, planes of 2D imaging data depicting the anatomical structures. The fitting can include iteratively transforming the planes to alter how the planes sit relative to each other. The transforming can be based on a comparison anatomical model (‘golden model’, or SSM). The fitting provides the 3D anatomical representation of the anatomical region.

In examples, the 2D imaging data informs, via image processing technique(s), for instance edge detection and/or gradient spikes as examples, plane transformations to ensure consistency between the plane transformations and what is depicted by the 2D imaging data. In this manner, the image processing techniques applied to source 2D data help keep the transformations reasonable/realistic, providing bounds on how extreme the transformations can be. Without these, one or more transformations could become unreasonable relative to what is depicted by the source 2D data.

The comparison anatomical model can be/include a 3D model that has structures (for instance, models of the individual bones in the anatomical region) having comparison properties (sizes/shapes/orientations) for the anatomical region. The comparison anatomical model can be provided with indications of how the comparison properties can vary based on a selected population of anatomical samples. By this is meant an indication of acceptable or reasonable deviations in how the properties might vary. These could be based on statistical averages or ranges, for instance. In examples, such indications could be encoded in the model itself, though indications could additionally or alternatively be provided as part of metadata and/or other data files with information about how the comparison model can reasonably vary.

A method could include building the AI model to process incoming 2D imaging data and provide annotations thereto. The building of such a model could include training the AI model on 2D segmented images that are pre-annotated as to landmarks of anatomy. As noted, landmarks could include features of bones, for instance bone centroids and proximal and distal ends of the bones. In examples, a training dataset of samples is built, with the samples including the 2D segmented images. To increase sample count, the building of the training dataset could include obtaining bilateral images and splitting the bilateral images into unilateral images that form some of the samples. Additionally or alternatively, rotation(s), flip(s), translation(s), and/or other image manipulations could be applied to existing samples of the training data set in order to produce additional samples for training.

Additionally or alternatively, a method is provided that obtains a 3D model of an anatomical region of a patient, the anatomical region having patient anatomical features, identifies at least one deformity of the patient anatomical features, where the at least one deformity is identified relative to target anatomical values for the patient anatomical features, wherein the target anatomical values include desired ranges into which anatomical measurements are to fall, and where the at least one deformity includes at least one of a rotational deformity and an angular deformity, and determines, using the anatomical measurements, at least one planar correction to make to at least one anatomical structure of the patient. The at least one deformity can include a plurality of deformities. A deformity of those could include an angular deformity, for instance an inter-anatomical angle between first and second anatomical features of the patient anatomical features. The first and second anatomical features could be, for instance, first and second metatarsals of the patient, such as in the case of a bunion.

Additionally or alternatively, the plurality of deformities can include the rotational deformity, such as (in the case of a bunion) a rotational deformity in a first metatarsal of the patient.

In examples, the identification of the at least one deformity includes selecting a geometric volume and applying the geometric volume to an anatomical feature, of the patient anatomical features, presented in the 3D model, using a property of the geometric volume, taken as a property of the anatomical feature, to determine a measurement of the anatomical measurements, and identifying a deformity, of the at least one deformity, based on the determined measurement.

The anatomical feature to which the geometric feature is applied could be/include a cross-section of a bone, or an articular surface of a bone, as examples.

In examples, a method includes obtaining imaging data of the anatomical region of the patient, and the obtaining of the 3D model includes generating the 3D model (such as described above) from the obtained imaging data.

In situations involving patient-specific hardware to facilitate the at least one correction to make to the at least one anatomical structure, the method can optionally generate a specification of the hardware. The specification can be generated based on (i) the representation of the patient anatomical features as provided by the 3D model and (ii) the determined at least one correction(s). The specification can include measurements/parameters tailored to the patient based on the representation of the patient anatomical features as provided by the 3D model and on the determined correction(s). In examples, the patient-specific hardware includes at least one hardware guide for guiding surgical procedure(s) to provide the correction(s) to make to the at least one anatomical structure. The hardware guide(s) can include, for instance, cut-guide(s) for cutting procedure(s).

Aspects of processing performed by software described herein can aid in correction of various conditions. Another example condition is metatarsus adductus, a deformity in which the forefoot turns inward. This deformity can optionally be considered and addressed in accordance with aspects described herein when addressing other condition(s) such as a Hallux valgus condition. Metatarsus adductus is sometimes exhibited in bunion cases.

The software can identify metatarsus adductus based on a 3D model generated as described herein. In a specific embodiment, this is detected by measuring a single angle, specifically the 2^{nd} tarsometatarsal angle (e.g., the angle between the longitudinal axes of the second metatarsal and intermediate cuneiform) (in the axial plane). If this angle exceeds a threshold, for instance −24 degrees, then metatarsus adductus is detected. There are varying options if metatarsus adductus is detected.

One option is to (virtually) correct the metatarsus adductus by making a rotation of the second and third metatarsals (in the axial plane) until the 2^{nd} tarsometatarsal angle is in a normative reference value range. This correction is expected to increase the 1^{st}-2^{nd} intermetatarsal angle (IMA) that is the subject of the Lapidus procedure, and therefore affect the extent of the IMA adjustment needed as part of the bunion correction. In conventional bunion treatment, no such consideration of a correction for a metatarsus adductus deformity is given as part of a Lapidus procedure. In this regard, aspects provide a surgical planning method for a Lapidus procedure that initially plans a correction for a metatarsus adductus deformity, and then plans a Hallux valgus correction based on the (corrected) metatarsus adductus deformity.

Another option is to take the metatarsus adductus condition into account in determining the amount of correction to perform as part of the Lapidus procedure to correct Hallux valgus. Under this approach, the second metatarsal is not initially rotated as was the case in the option above; instead, the planning initially rotates the first metatarsus to an overcorrection (relative to a normative reference value) as part of addressing the Hallux valgus, such that the end result addresses the metatarsus adductus deformity as well as the bunion. Additionally, the amount of correction is limited by the distance between the 1st and 2nd metatarsal head, for instance the software prevents their overlap, which may limit the amount of correction.

In specific embodiments, the surgical planning software identifies and notifies the user if metatarsus adductus is detected, for instance based on the 2nd tarsometatarsal angle (axial plane) exceeding a threshold. In specific examples, the threshold is −24 degrees. The user can be given an option to continue with Lapidus procedure planning, for instance an option to continue without correction(s) to address the metatarsus adductus and an option to continue with correction(s) to address the metatarsus adductus.

In examples, selection of the option of not addressing/correcting the metatarsus adductus results in the first ray of the foot (i.e., segment composed of the first metatarsal and first cuneiform bones) being overcorrected, meaning that the 1^{st}-2nd intermetatarsal angle (axial plane) is corrected to its normative reference determined as if the 2nd metatarsal was in its normal position defined by the normative reference value of the 2nd tarsometatarsal angle (axial plane). FIG. 21 shows an example of Lapidus virtual correction output based on selection of the option to not address the metatarsus adductus. In this example, the planned post-operative 1st-2nd intermetatarsal angle (axial plane) is 5.7 degrees, the 2nd metatarsal stays in its pre-operative position, and the overcorrection is limited so that first ray bones cannot overlap with the bones on the 2nd ray (i.e., segment composed of the second metatarsal and second cuneiform bones).

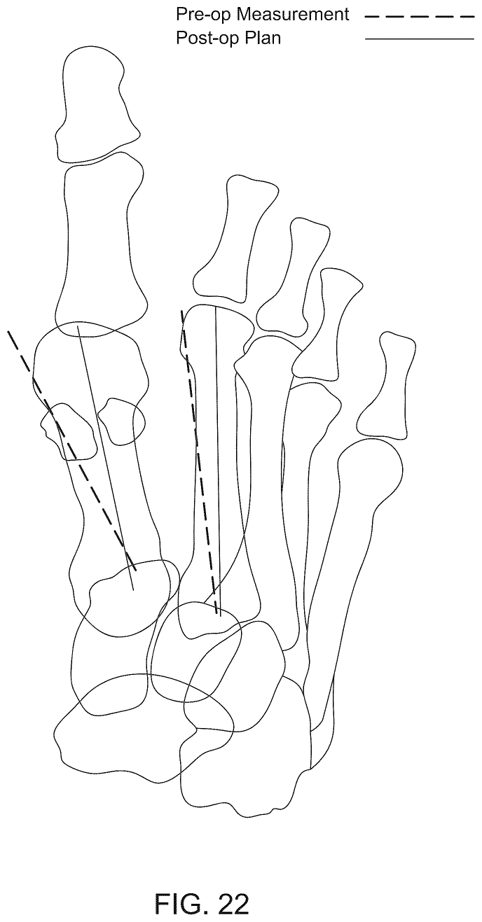

In examples, selection of the option of addressing/correcting the metatarsus adductus results in the 2nd ray and 3rd ray (i.e., segment composed of the third metatarsal and third cuneiform bones) being rotated so that the 2nd and 3rd tarsometatarsal angles (axial plane) match to their normative reference values. The Lapidus arthrodesis procedure can be performed normally after that, though as mentioned previously the IMA correction performed as part of the Lapidus procedure may be different than if the metatarsus adductus were not being addressed. FIG. 22 shows an example of Lapidus virtual correction output with the option of the metatarsus adductus being addressed. The corrections for the 2nd and 3rd rays are shown for visualization. With this example case, the planned post-operative 1st-2nd intermetatarsal angle (axial plane) is the default reference value 11.4 degrees.

An additional consideration can be given to plantar flexion, specifically plantar flexion of the first metatarsal, when making corrections to anatomy of the foot. A resection at the tarsometatarsal (TMT) joint results in a shortening in the first ray since the 1^{st} metatarsal is moved in the posterior direction toward the medial cuneiform. Because the first metatarsal is angulated to the floor plane, the metatarsal head elevates from the floor plane.

It may be desired to avoid this by keeping the first metatarsal head elevation constant, substantially the same as its elevation prior to the surgical procedure being performed, or another elevation. To this end, the software can simulate, based on the generated 3D model of patient anatomy, the position of the first metatarsal head on the patient if the deformity that is the subject of the surgical procedure did not exist. In other words, the software can determine where the first metatarsal head would sit for this patient if the angles (or other markers of deformities) were in healthy range(s). In a particular example, the software simulates a correction of the sagittal IMA and rotation without any resection to the TMT joint, and captures the elevation of the head in this ‘corrected’ position. The software can then move onto next step(s) of the planning procedure—for instance planning the resections—and use that first metatarsal head elevation as a constraint/parameter to the resection planning.

Aspects described herein can additionally be used to facilitate generation of simulated weight-bearing computed tomography (WBCT) models. CT imaging data is typically collected with the patient in a non-weight-bearing position, for instance while the patient is lying down. In contrast, aspects can be used to create a simulated WBCT model by pairing a 3D model of patient anatomy, for instance one that was generated in accordance with aspects described above, with indications of the locations of patient anatomical components when in a weight-bearing position (for instance, created based on one or more x-rays of the anatomy, such as the foot, foot in a weight bearing position).

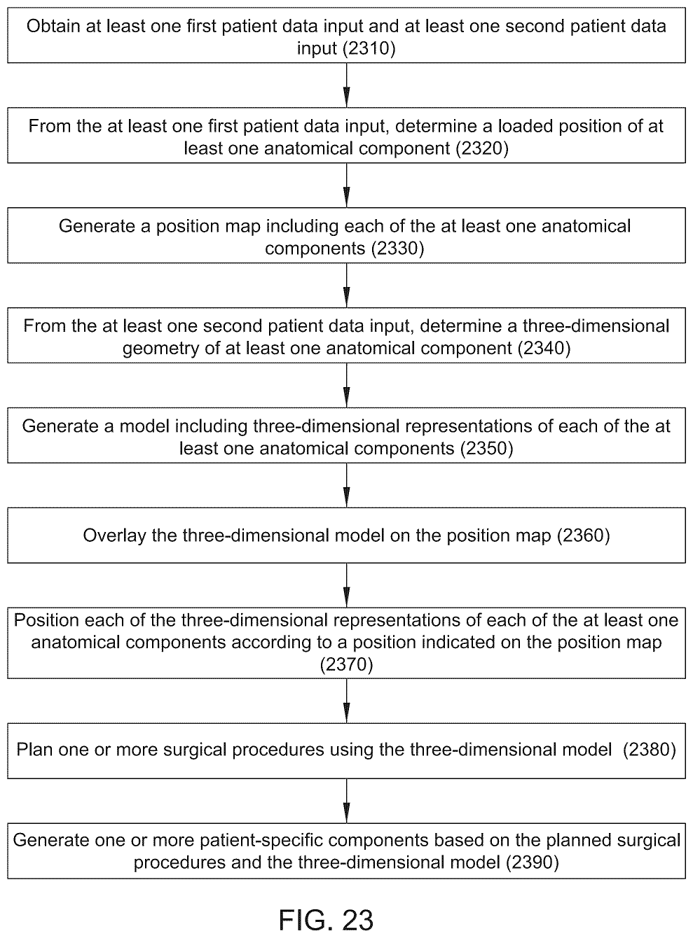

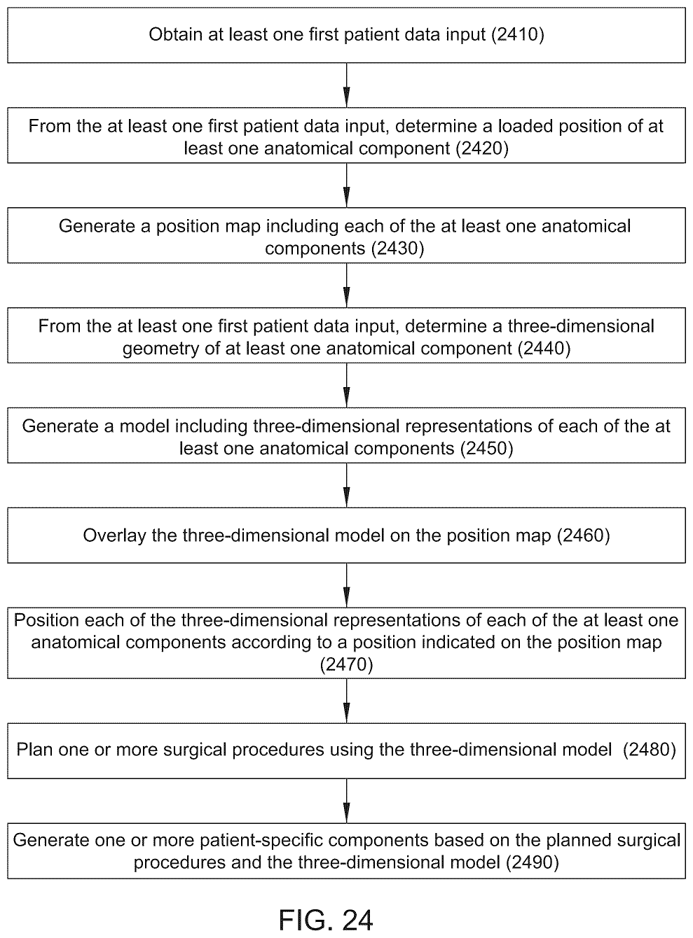

Examples processes for simulated WBCT model generation are depicted by FIGS. 23 and 24, and explained as follows. Referring initially to FIG. 23, the process obtains (2310) at least one first patient data input and at least one second patient data input. In embodiments, the first patient data input is weight-bearing x-ray(s) image data (WBXR), for instance x-ray images/image data in two planes, though other forms of radiographs, including multiple digitally reconstructed radiographs for instance, may be used. In embodiments, the second patient data input is traditional (non-weight-bearing) CT image data. However, WBCT data may be input into this process as well, for example if a weight-bearing aspect of the WBCT may be flawed or inaccurate.

From the at least one first patient data input, the process determines (2320) a loaded position of at least one anatomical component. For instance, the process determines a loaded position of each bone (each of the anatomical components) exhibited in the first patient data input when in a weight-bearing (“loaded”) position in three dimensions. While in some examples there may be an indication of the angle at which the superior view WBXR was taken, for instance to account for it not being perfectly positioned relative to the target anatomy, there are no strict requirements for this. A wide range of input views may be supported, and in situations involving a non-supported view, for instance if there is too large an angular deviation from an expected view angle, this could be dealt with in various semi-automated or fully-automated ways. For instance, an AI model could be trained on such views to learn how to work with them.

The process also generates (2330) a position map that includes each of the at least one anatomical component. In specific examples, this position map is a superior view showing the location of each of the anatomical components seen in the WBXR. In this aspect, the anatomical components can be identified/segmented manually or automatically.

From the at least one second patient data input, the process determines (2340) a three-dimensional geometry of at least one anatomical component. For instance, 3D geometry including volume, shape, surface topology, etc. is determined for each of the anatomical components (e.g., bones) exhibited in the second patient data input. From the 3D representations available from the second patient data input, the process generates (2350) a 3D model that includes three-dimensional representations of each of the at least one anatomical component. The 3D model may be generated by the software to provide the form of 3D objects that the software uses to manipulate, measure, etc. the 3D geometries reflected in second patient data input. In examples, the generating (2350) is performed as described above, for instance with reference to path 201a of FIG. 2, and the second patient data (e.g., CT data) is either manually or automatically segmented to create the model. It is noted that exact positioning of the anatomical components (e.g., bones) is not critical at this point. The process overlays (2360) the three-dimensional model on the position map and positions (2370) the three-dimensional model by positioning each of the three-dimensional representations of each of the at least one anatomical component according to a position indicated on the position map, for instance by lining up each of the 3D representations of the individual anatomical components (e.g., bones) with the matching positions thereof based on the WBXR. In this aspect, each individual 3D anatomical model (e.g., bone model) is positioned relative to the position indicated for that bone on the position map, matching an ID of the 3D component to a position with a complementary ID.

The process then plans (2380) one or more surgical procedures using the positioned 3D model. For instance, the planning includes identifying axes, measuring angles, comparing measurements to normal values, etc., Finally, the process generates (2390) one or more patient-specific components based on the surgical plan (planned one or more surgical procedures from 2380) and the positioned 3D model. Examples of such patient-specific components include guides, implants, instruments, etc. based on the surgical plan.

FIG. 24 depicts an example process for simulated WBCT model generation in which the second patient data input, for instance traditional (non-weight-bearing) CT image data, is not required. Instead, first patient data input, for instance weight-bearing x-ray(s) image data, and more specifically x-ray images/image data in two planes as an example, is obtained (2410), though other forms of radiographs may be used. From the at least one first patient data input, the process determines (2420) a loaded position of at least one anatomical component. For instance, the process determines a loaded position of each bone (each of the anatomical components) exhibited in the first patient data input when in a weight-bearing (“loaded”) position in three dimensions. The process also, in this example, generates (2430) a position map that includes each of the at least one anatomical component. Generation of the position map is used in this example, as in the example above, but is not strictly necessary. More generally, positions on the WBXR are identified and the 3D model components are assigned to corresponding positions.

From the at least one first patient data input, the process determines (2440) a 3D geometry of at least one anatomical component. For instance, 3D geometry including volume, shape, surface topology, etc. is determined for each of the anatomical components (e.g., bones) exhibited in the first patient data input, then the process generates (2450) a model that includes three-dimensional representations of each of the at least one anatomical component, overlays (2460) the 3D model on the position map and positions the 3D model by positioning each of the three-dimensional representations of each of the at least one anatomical component according to a positions indicated on the position map, for instance by lining up each of the 3D representations of the individual anatomical components (e.g., bones) with the matching positions thereof based on the WBXR. In this aspect, each individual 3D anatomical model (e.g., bone model) is positioned relative to the position indicated for that bone on the position map, matching an ID of the 3D component to a position with a complementary ID.

The process then plans (2480) one or more surgical procedures using the positioned 3D model. For instance, the planning includes identifying axes, measuring angles, comparing measurements to normal values, etc., Finally, the process generates (2490) one or more patient-specific components based on the surgical plan (planned one or more surgical procedures from 2380) and the positioned 3D model. Examples of such patient-specific components include guides, implants, instruments, etc. based on the surgical plan.

Further details of aspects described herein, and additional aspects, are presented within the context of a description of example surgical planning software (“software”) and functionality thereof. The surgical planning software can be intended for use by healthcare professionals, for instance by orthopedic professionals to assist in the characterization of anatomical structures of various anatomy, such as the foot and ankle, using three-dimensional mathematical modeling and radiographic measurements. Combined information from structural models and radiographic measurements can be used for diagnostic and treatment planning purposes. Various different medical imaging types may be used as input to the software, for instance x-ray, CT, weight-bearing plain film x-ray, and weight-bearing CT (WBCT), as examples.

The software can be used by orthopedic (and other) healthcare professionals for diagnosis and surgical planning in a hospital or clinical environment. The software can provide the following and other functionality for the user:
Visualization reports of three-dimensional (3D) mathematical models and measurements of anatomical structures (e.g., feet and ankles);
Measurement templates containing radiographic measures of anatomy of the anatomical structures; and
Surgical planning for visualization of anatomical three-dimensional structures, radiographic measures and three-dimensional models of orthopedic fixation devices and surgical guides.

In examples, a visualization report containing measurements of anatomical structures can be used for the diagnosis of orthopedic healthcare conditions. The surgical planning application containing the visualizations of the three-dimensional structural models, orthopedic fixation device models, and surgical guide models, combined with the measurements, can be used for the planning of treatments and operations to correct orthopedic healthcare conditions. The surgical planning application/software output can also be used for designing patient-specific orthopedic surgical instruments.

In some embodiments, the software is provided as a web application without specific hardware requirements, in which the software is presented and used via a web browser.

By way of non-limiting example, the software can accept computed tomography (CT) and cone beam computed tomography (CBCT) three-dimensional (3D) imaging data in DICOM format. The quality of the visualization and the outputs from the software could be dictated by the quality and resolution of the original DICOM image series. Example CBCT imaging parameters are presented in Table 1:

TABLE 1

| Parameter | Value |
|---|---|
| Imaging Protocol: | Standard |
| Projections: | 400 |
| Filtering: | None |
| Voxel size: | 0.4 × 0.4 × 0.4 mm³ |
| Tube voltage: | 96 kV (KVP, peak kilo voltage output of the |
| | x-ray generator used) |
| Tube current: | 6.3 mA (x-ray tube current in mA) |
| Pulse length: | 20 ms |
| Field-of-view: | All metatarsals, phalanges, and bones of the |
| | midfoot completely within view. For the 3D |
| | workflow, the field-of-view is limited to the |
| | distal portion of the foot, containing |
| | approximately one third of distal tibia and |
| | fibula (390 mm proximally measured from |
| | the sole of the foot). |
| Additional Notes: | DICOM image series reconstructed with |
| | hard kernels and with even slice spacing. |
| | The effective slice thickness may be up to |
| | 0.75 mm, above 1 mm is not recommended. |
| | Images with slice thickness or pixel spacing |
| | below 0.4 mm are resampled up to 0.4 mm. |

The software could accept native x-ray images. Dorsoplantar and lateral views may be used to construct three-dimensional (3D) digital models from planar x-ray images. Example native x-ray imaging parameters are presented in Table 2:

TABLE 2

| Parameter | Value |
|---|---|
| Source-image receptor | 1000 mm |
| distance (SID): | |
| Patient position: | All images should be taken under |
| | weightbearing, with the patient standing. |
| | The pose must remain constant between the |
| | scans. |
| | Dorsoplantar series: AP projection with X- |
| | ray tube rotated 15° frontally to allow |
| | weight-bearing, beam centered on the base |
| | of 3^{rd} Metatarsal |
| | Lateral series: True lateral view, beam |
| | centered on the base of the metatarsals. |
| Exposure: | 3-6 mAs, 50-60 kVp |
| Field-Of-View: | Dorsoplantar series: All metatarsals, |
| | phalanges, and bones of the midfoot |
| | completely within view. The talus and the |
| | calcaneus should also be within view even |
| | if occluded by distal aspect of tibia and |
| | fibula. |
| | Lateral series: All metatarsals, phalanges, |
| | and tarsal bones completely within view. |
| Other Projections: | Unsupported orientations, such as coronal |
| | view, should not be submitted. |

Certain DICOM tags, with conformance to NEMA PS 3.1-3.20 2021e, be present on images used for analysis. If a tag is missing, the DICOM image series may be considered invalid, and the software may be unable to use the image series. Example tags are listed in Table 3

TABLE 3

| | | Required tags by imaging modality | | | |
|---|---|---|---|---|---|
| | | | Enhanced | | |
| Tag | Name | CT | CT | DX | CR |
| (0008, 0016) | SOP Class | x | x | x | x |
| | UID | | | | |
| (0028, 0004) | Photometric | x | x | x | x |
| | interpretation | | | | |
| (0028, 0010) | Rows | x | x | X | X |
| (0028, 0011) | Columns | x | x | x | X |
| (0028, 0100) | Bits | x | x | x | X |
| | Allocated | | | | |
| (0028, 0102) | High Bit | x | x | x | x |
| (0028, 0030) | Pixel | x | x | x*) | x*) |
| | Spacing | | | | |
| (0020, 0037) | Image | x | x | | |
| | Orientation | | | | |
| | (Patient) | | | | |
| (0020, 0032) | Image | x | x | | |
| | Position | | | | |
| | (Patient) | | | | |
| (0028, 0008) | Number of | | x | | |
| | Frames | | | | |
| (0018, 1164) | Imager Pixel | | | x | x |
| | Spacing | | | | |

*)Required if the image has been calibrated

Example software measurement range and precision (for feet/ankle imaging area) are as follows:
Range: ±180°, ±500 mm (foot and ankle imaging area)
Precision: 0°, 0 mm (deterministic automatic image analysis)

In examples, the software includes a web user interface component for sending images for analysis and reviewing output report(s), and a cloud-based service providing/outputting the analysis, measurements, and a surgical plan.

Example specifications for connection requirements to connect to the cloud service are: 1. Protocol: Hypertext Transfer Protocol Secure (HTTPS); 2. Encryption: Transport Layer Security (TLS); 3. API domain: [a specified domain to the service]; 4. Port: 443 (TCP).

The software can have a data management feature for downloading existing case reports, as described further below.

Example cybersecurity controls of the software are presented in Table 4:

TABLE 4

| | |
|---|---|
| User authentication: | Microsoft Azure Active Directory (AD) |
| Protocol: | Hypertext Transfer Protocol Secure (HTTPS) |
| Encryption: | Transport Layer Security (TLS) |
| Data encryption: | Encrypted data at rest |
| Firewall: | Local IT Firewall configuration applies |
| Anti-virus policy: | Computers using the software should have |
| | up-to-date virus and malware protection |

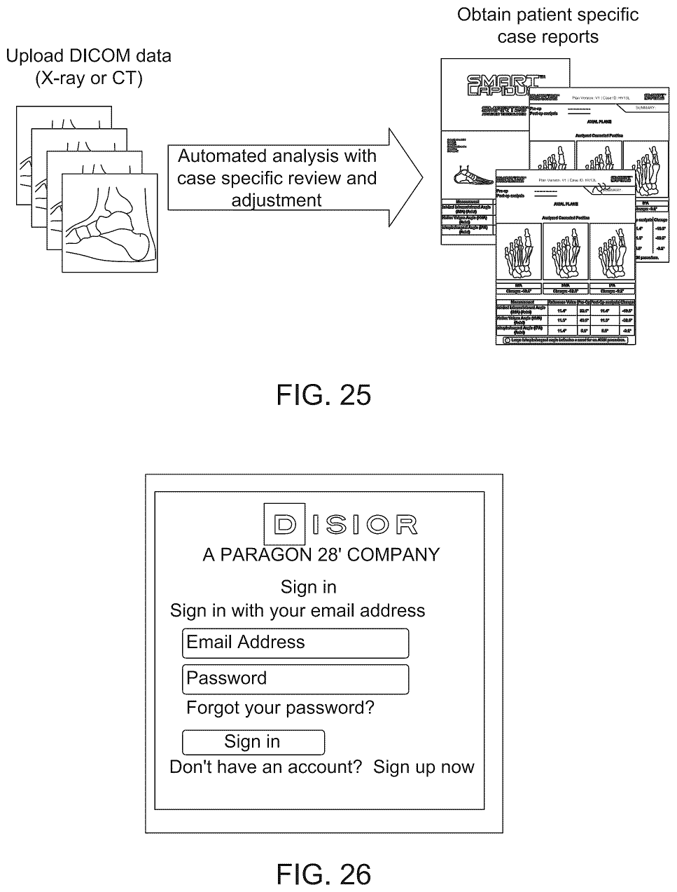

FIG. 25 depicts an example overall workflow for surgical planning in accordance with aspects described herein, which includes upload/provision of image data, for instance DICOM data in the form of x-ray or CT images, automated analysis with case-specific review and adjustment, and patient-specific case reporting for the user of the software. The workflow of the software could be fully automatic from the point of image upload, through the analysis reporting and the surgical plan delivery. The workflow can include of the following steps, as examples:
1) User authentication (sign in with email and password);
2) Case management (old case selection and report download);
3) Start a new case;
4) Image type (post-op/pre-op) and laterality selection;
5) Start analysis;
6) User confirmation for analysis;
7) Adjust target values (optional);
8) Download report;
9) Save and export results.

Further details of the 9 steps above are as follows.

FIG. 26 presents an example interface for user authentication after opening/loading the software (e.g., the web app) in a browser. The user is prompted to enter the user's email address and password.

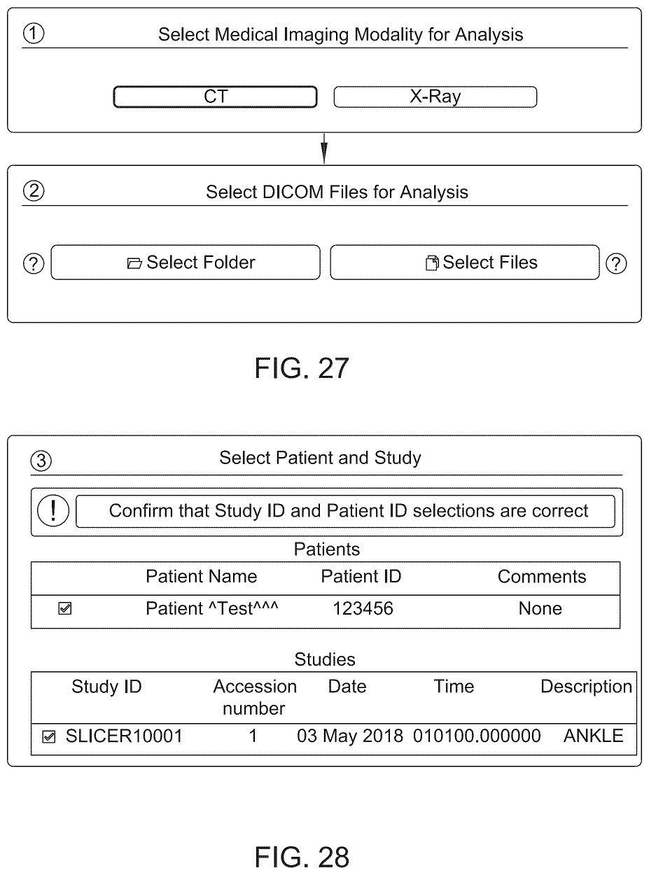

FIG. 27 presents an example interface for case management, in accordance with aspects described herein. The user can start a new case by selecting the New Case button, or select in the “Your Previous Cases” an existing case to view/download. Selection of an existing case presents details in the Case Information interface area, and enables the user to Open the report or update the analysis, in this example.

Also shown is a ‘case in progress’ dialog area indicating when the case was started and a status of the case (e.g., that analysis of the case is updating).

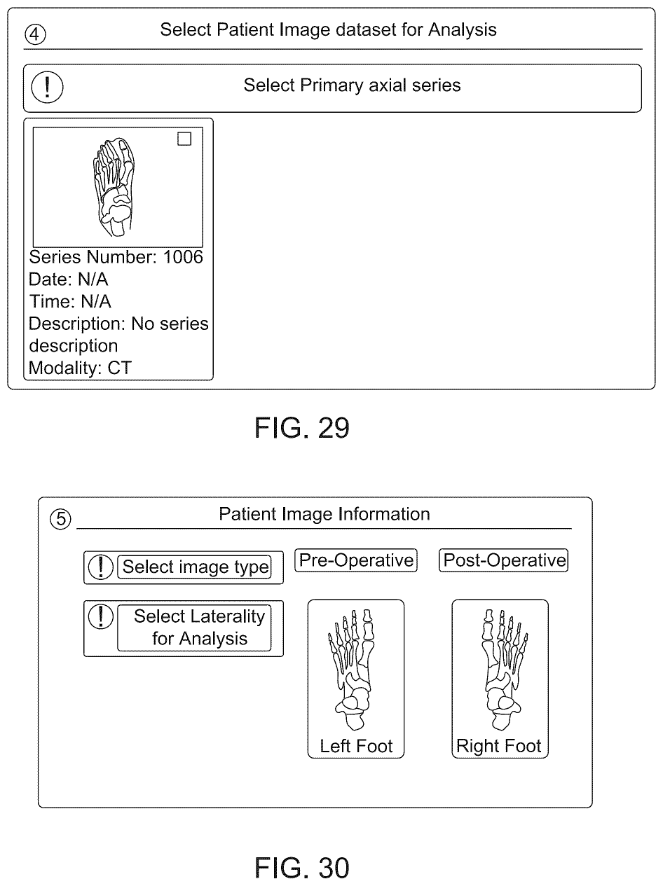

FIGS. 27-29 depict example interfaces for starting a new case. If selecting to start a new case, the user selects a medical imaging modality (e.g. as between CT and X-RAY) and related DICOM imaging folder/files (see FIG. 27). In a two-dimensional (2D) case using a native planar x-ray modality, images for Lateral and Dorsoplantar views are provided by the user. In a three-dimensional (3D) case, (at least) one image series is needed. The user uploads the DICOM files from the selected folder. For folders containing multiple DICOM image series, all series can be listed in the software with the associated, respective metadata and image preview displayed (see FIGS. 28, 29). The user selects the DICOM image series (for 3D workflow) or the DICOM images (for 2D workflow) for analysis from the list generated. If the folder contains a large number of DICOM series, then it could take longer to load.

The workflow proceeds to image type and laterality selection (see FIG. 30) at which the user chooses the image type (pre-operative or post-operative) and the laterality based on the pictograms of the anatomy (e.g., left or right foot).

The analysis report as shown below in connection with downloading/viewing the report can be obtained for pre-operative and post-operative cases, and can include the following data (in this example) based on the selected image type:

| Pre-operative case: | Post-operative case: |
|---|---|
| 1) Case ID | 1) Case ID |
| 2) Username | 2) Username |
| 3) Date and time | 3) Date and time |
| 4) Foot laterality | 4) Foot laterality |
| 5) Image modality and type | 5) Image modality and type |
| 6) Pre-operative foot position | 6) Analyzed foot position |
| 7) Corrected foot position | 7) Measurements |
| 8) Measurements in pre-operative condition | |
| and after correction | |
| 9) Amount of total correction for | |
| measurements | |
| 10) Intra-operative plan with correction | |
| steps | |

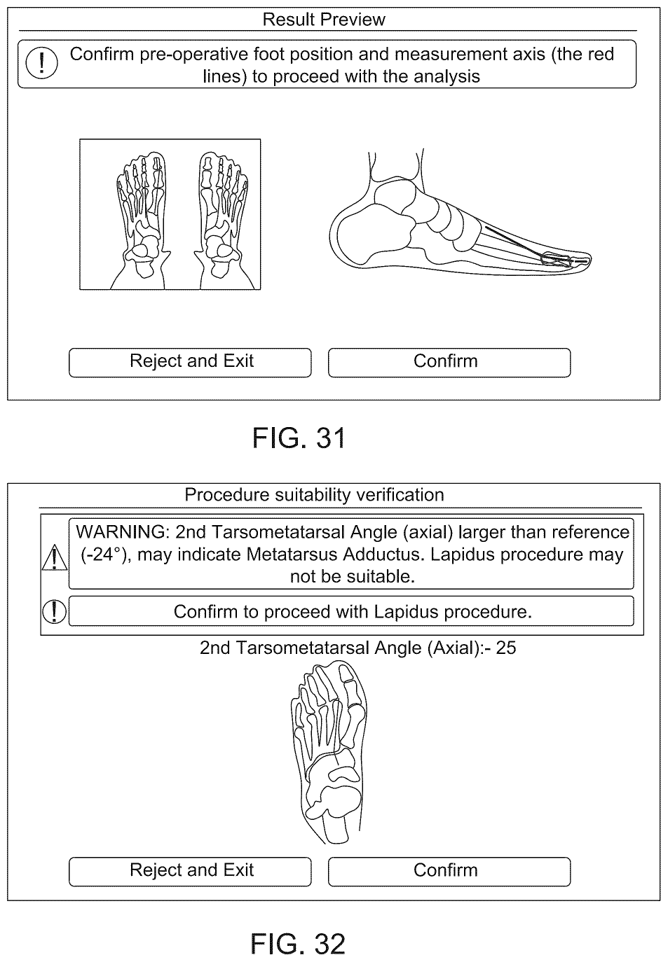

When the workflow starts analysis, the software can prompt the user to confirm pre-operative foot position and measurement axis to user in proceeding with the analysis, as shown in FIG. 31.

If a possible Metatarsus Adductus is detected, for instance if the software detects a 2nd Tarsometatarsal Angle (Axial) larger than a predefined reference (such as −24°), then an additional confirmation (shown in FIG. 32) may be presented to continue with (confirm) Lapidus procedure planning (press confirm button), or reject the planning if the user does not desire to continue with Lapidus procedure planning (press Reject and Exit-button).

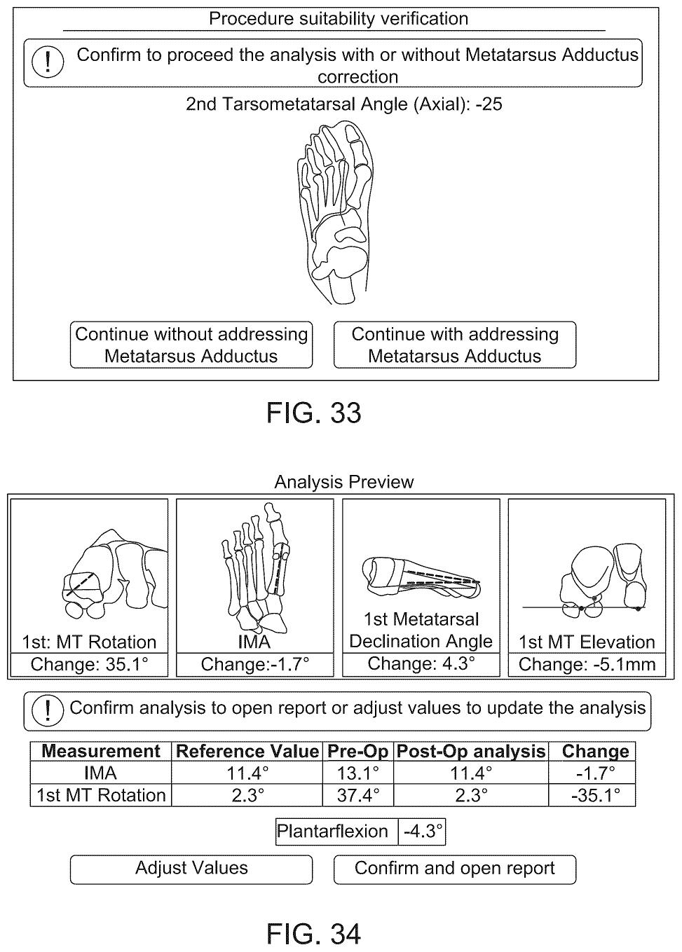

If the user desires to continue with the Lapidus procedure planning (selecting the Confirm button in the interface of FIG. 32), then the user is presented with an interface (example shown in FIG. 33) to make a selection as between (i) continuing the analysis with correcting the Metatarsus Adductus condition and (ii) continuing the analysis without correcting the Metatarsus Adductus condition. These options are described further below.

Based on the user's selections, a correction preview may be presented. An example interface providing a correction preview is presented in FIG. 34. The user can confirm and open a report by selecting the appropriate button, or can choose to adjust target values by selecting the appropriate button.

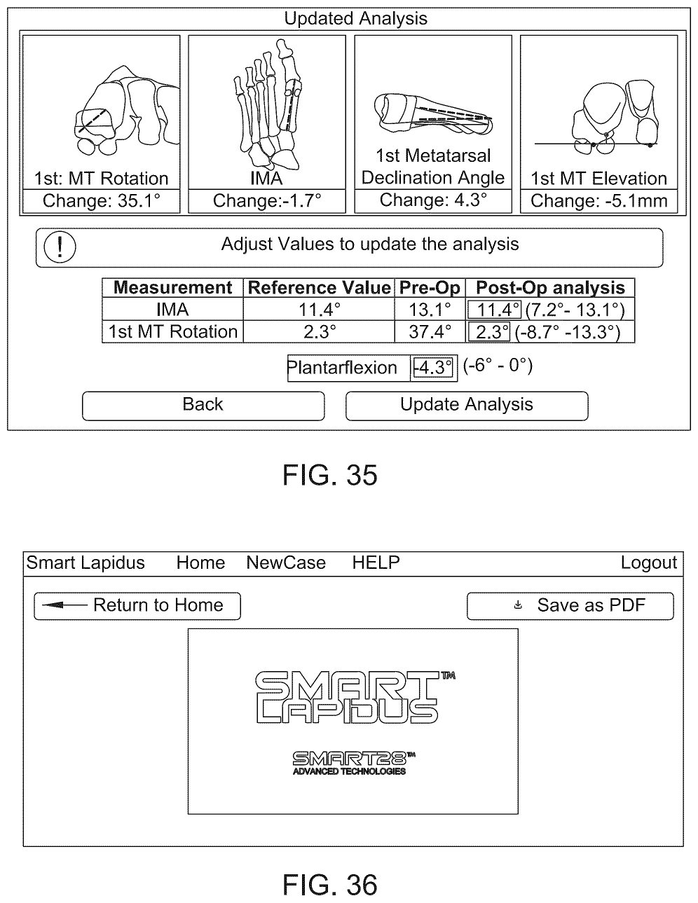

If the user selects to adjust target values, the user can enter new values in input boxes (see FIG. 35) which may be outlined (with green in this example) or otherwise highlighted, to provide updated targets and select the Update Analysis button. As a result, the software will update its analysis and present a new correction preview (FIG. 34).

If the user confirms to open the report (FIG. 34), the workflow proceeds to a report view/save interface as shown (partially) in FIG. 36, where the user can download/save the report by clicking the Save as PDF button, or simply view the report in the current interface.

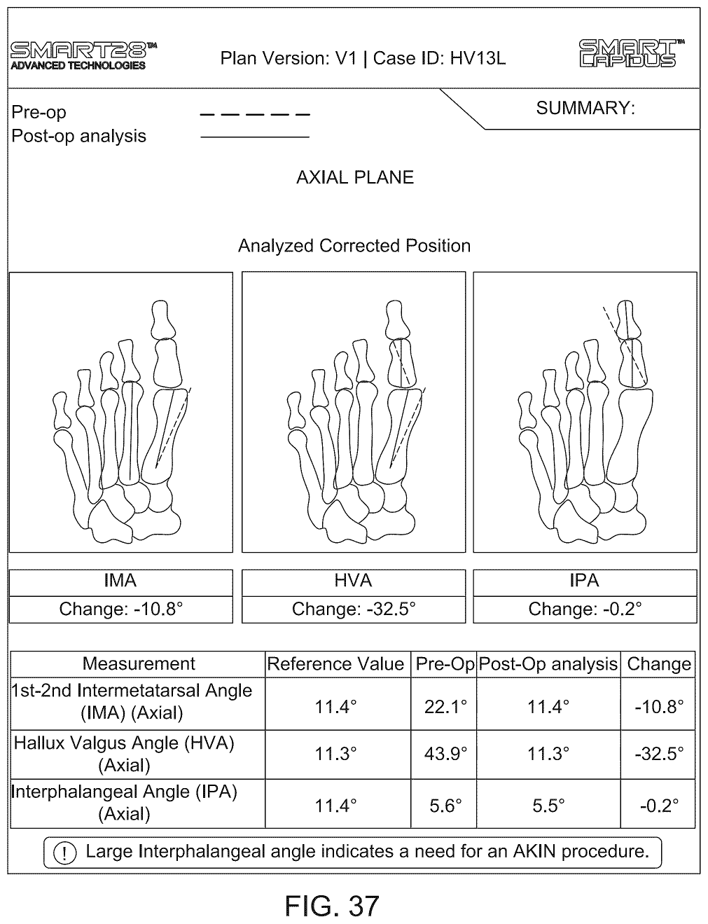

An example summary page of a report is shown in FIG. 37. The software also enables the user to save and export results. For instance, the analysis from the software can be exported for review outside of the software, and also saved in a (proprietary) format for later review within the software at a later time. The analysis report can also be saved in the software.

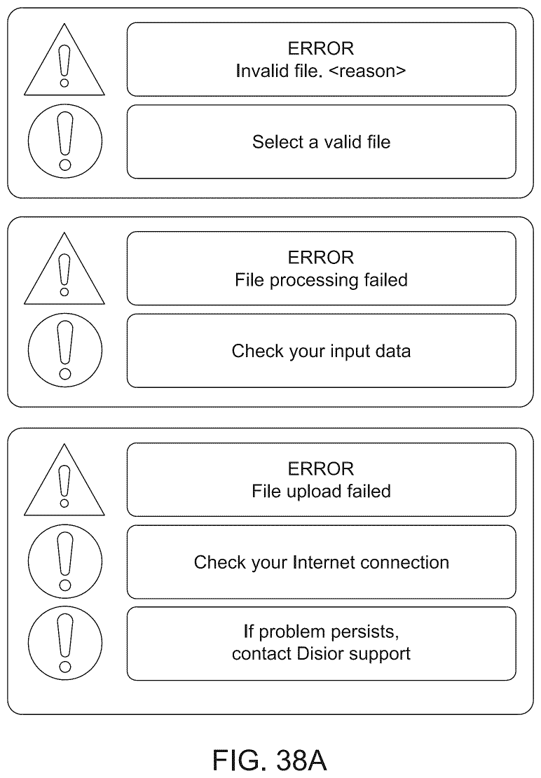

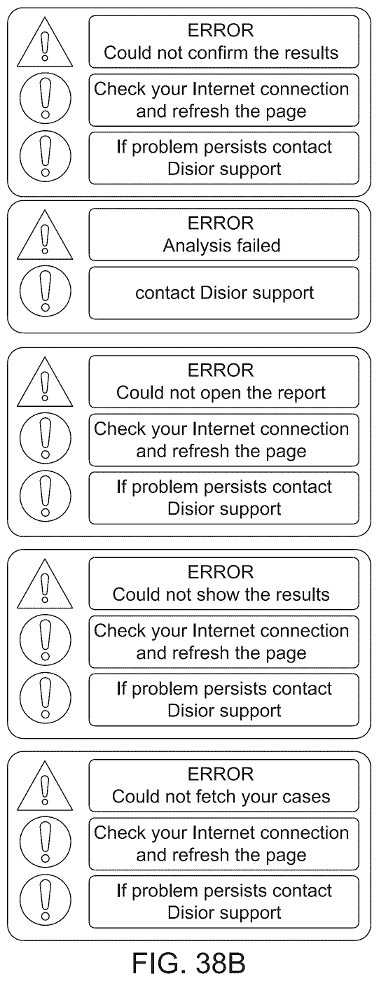

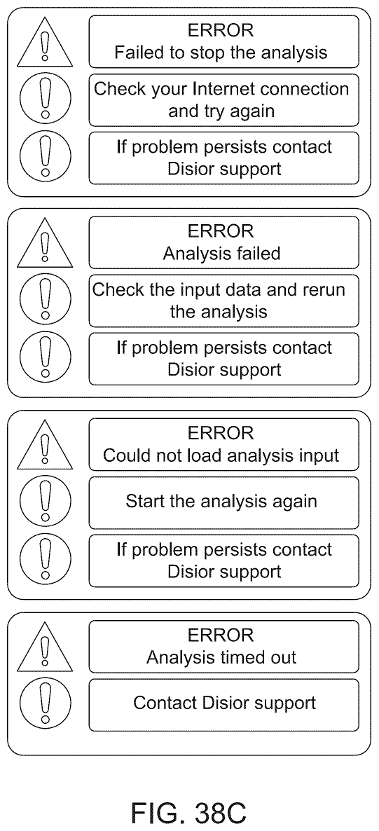

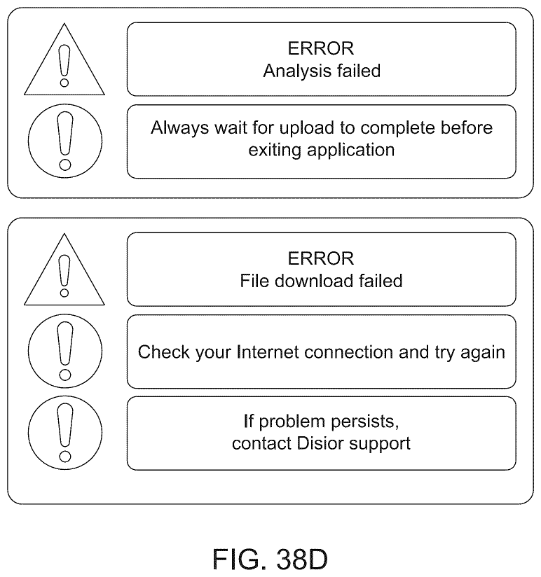

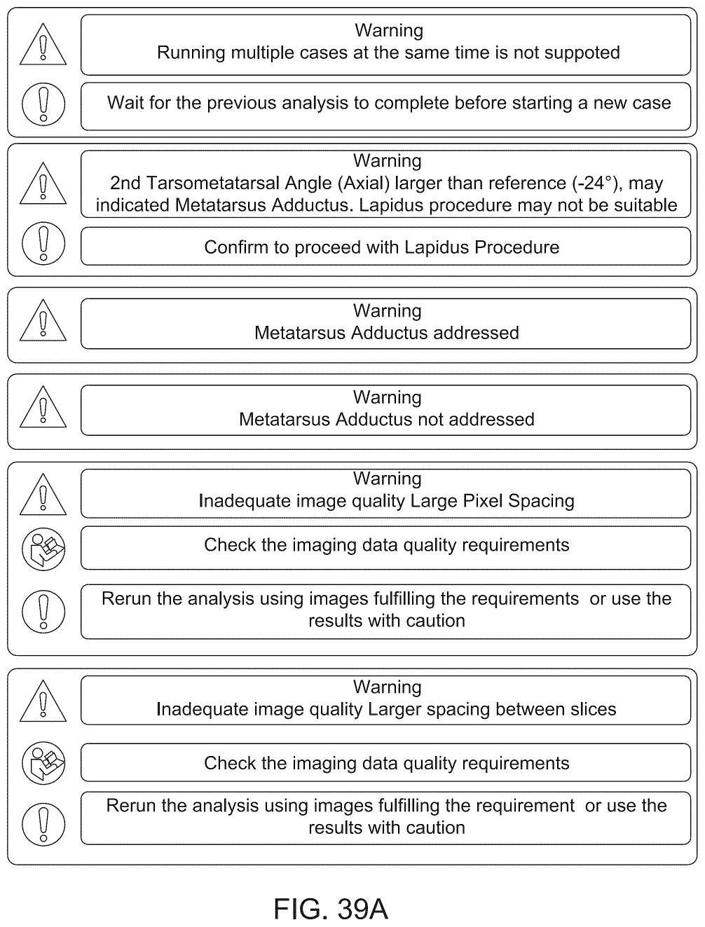

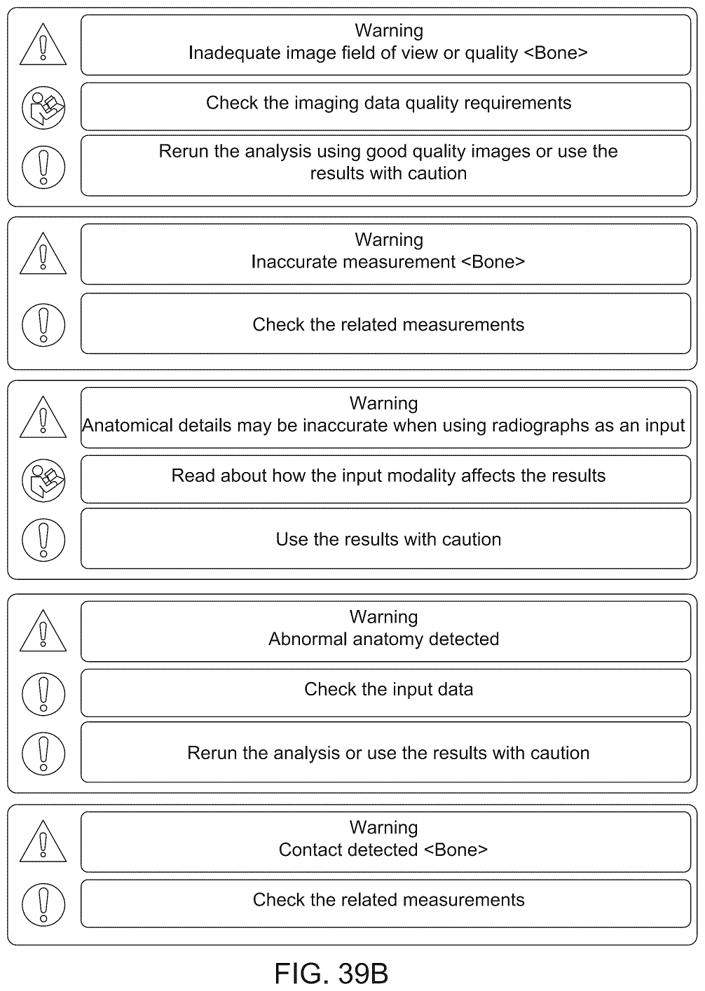

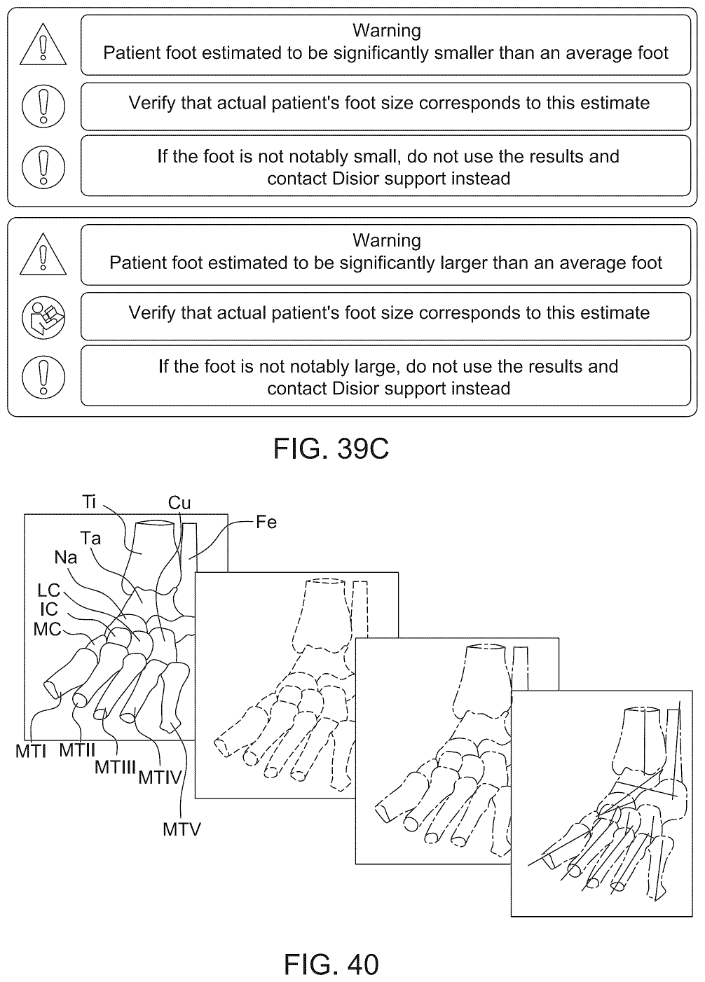

Example error messages that may be presented to a user of the software are shown in FIGS. 38A-38D. Example warning messages that may be presented to a user of the software are shown in FIGS. 39A-39C.

The software, as part of its analysis, can perform automated anatomical measurements, for instance to automatically calculate distances and angles between bones and specific landmarks to reliably evaluate human anatomy in three-dimensions (3D). Some principles and processes behind different measurement types, and some measurements available per anatomical area are now presented.

Automated and accurate bone tissue segmentation and shape analysis of the software enables, for instance, calculation of inter-bone angles and distances between clinically relevant landmarks in a patient-specific coordinate system, for example, to quantify dislocations and malformities such as hallux valgus. Measurements available for the foot and ankle area can be calculated to define or identify, for instance, forefoot deformities, and Hallux valgus. The following describes such measurements and the axes, landmarks, and surfaces used for calculation thereof. Various model types are shown in FIG. 40 to illustrate example color-coded, solid bone, and transparent overlapping bone (with indications of bone axes).

For foot and ankle measurements, angle measurements may be calculated based on 2D projections of 3D axes. 2D projection planes may be deduced from the imaging device's patient coordinate system, and measures may be shown with + or − signs. Meary's angle (sagittal) uses a sign negative towards pes planus and positive sign towards pes cavus.

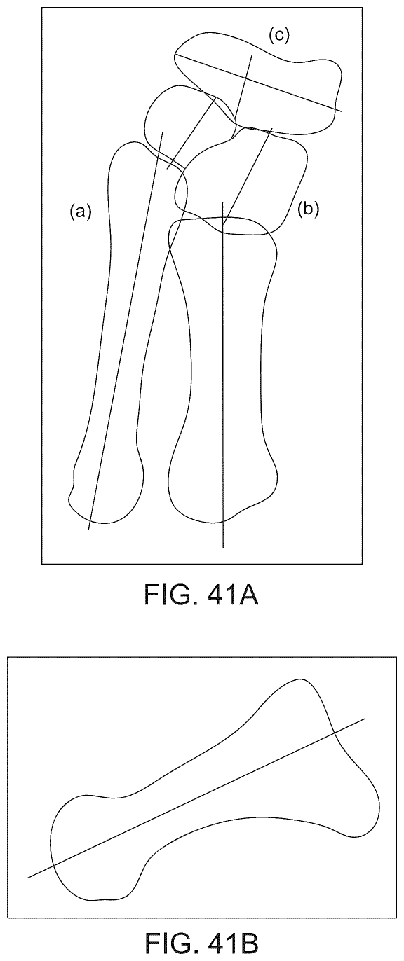

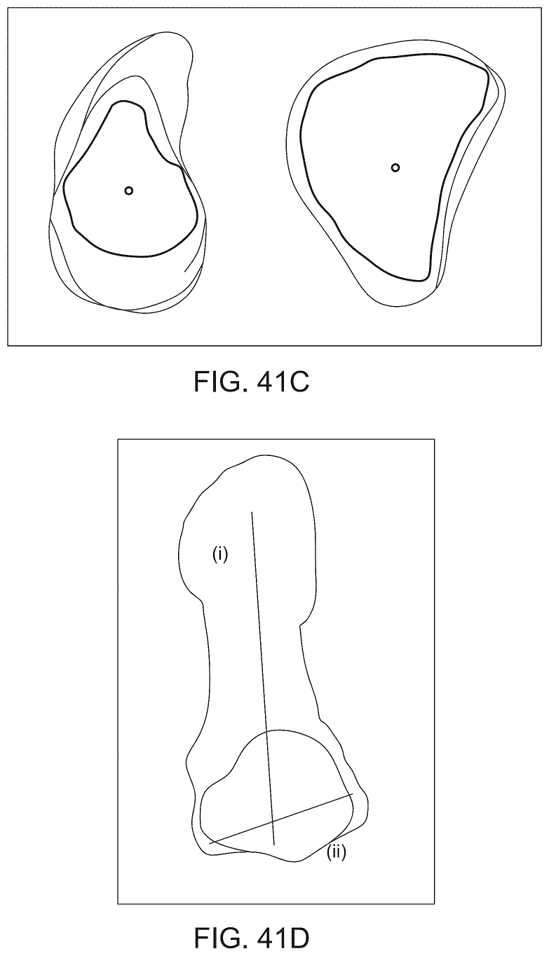

With respect to bone axes, and referring to FIGS. 41A-41D, the original analysis from the solver can define axes differently for each category of bone (a-c). Referring to FIG. 41B, elongated bones (category a), for instance Metatarsal and Proximal phalange bones, uses a longitudinal axis. The software can scan the bone and determine its cross-section at various locations. A weighted center point is computed for each cross-section, then robust line-fitting is used to find a straight-line representative for the center points of the cross-sections. Referring to FIG. 41C and category (b), an example of which is the Medial cuneiform, the software determines a cuneiform anteroposterior axis which extends through the navicular-cuneiform articular surface weighted center point, and through the cuneiform-metatarsal articular surface weighted center point. Referring to FIG. 41D depicting the 1st Metatarsal (a), the software determines (i) a longitudinal axis as for the elongated bone, and (ii) distal mediolateral axis, which is the axis of a virtual cylinder aligned with the 1st metatarsal distal articular surface.

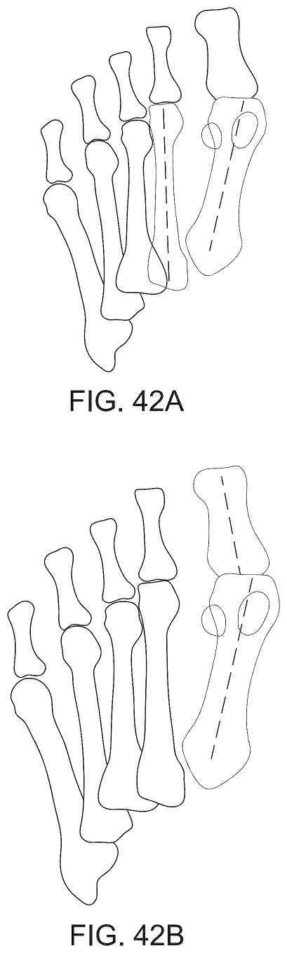

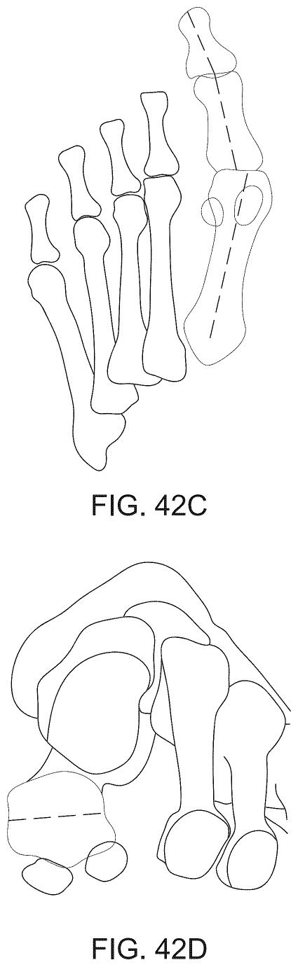

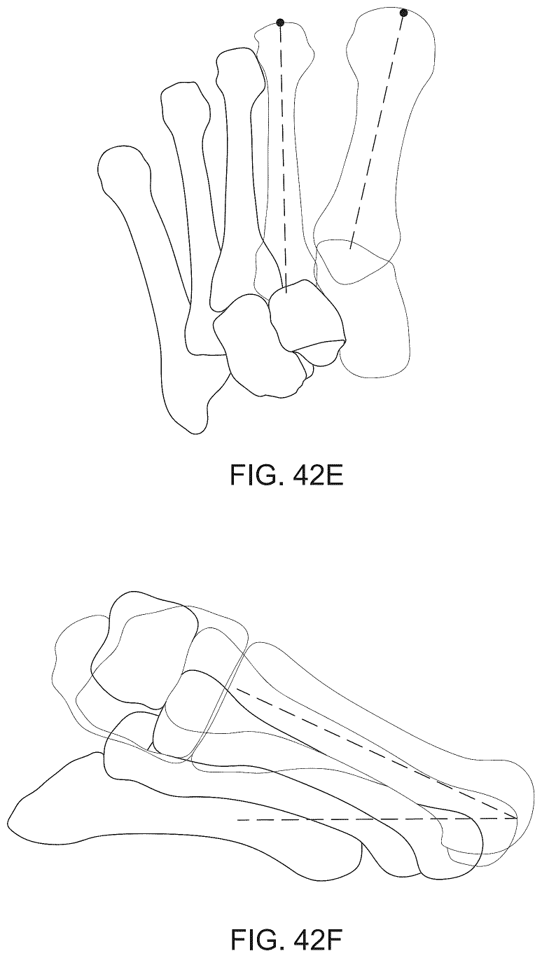

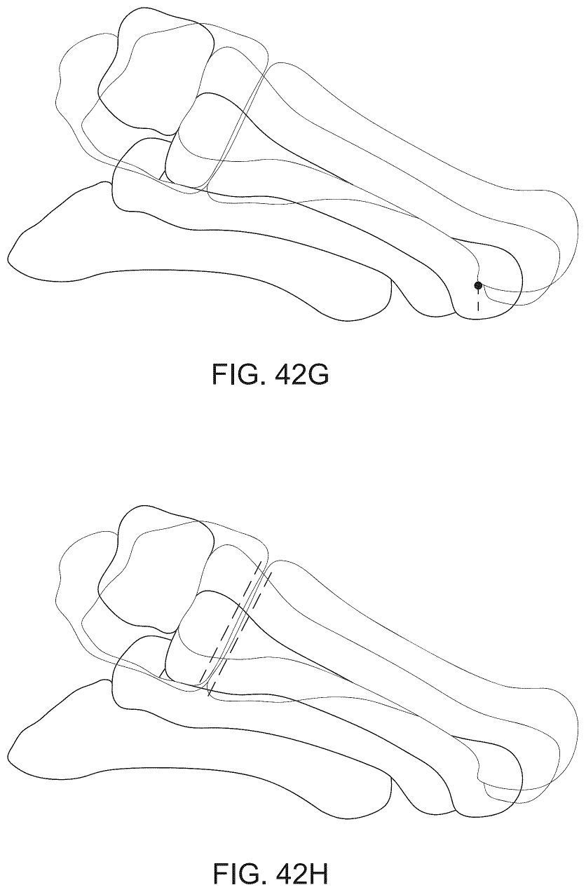

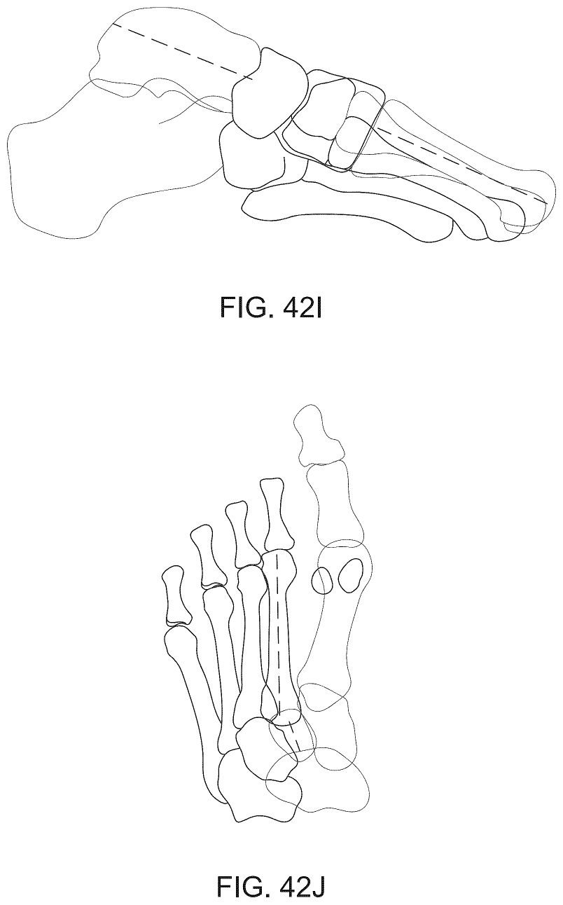

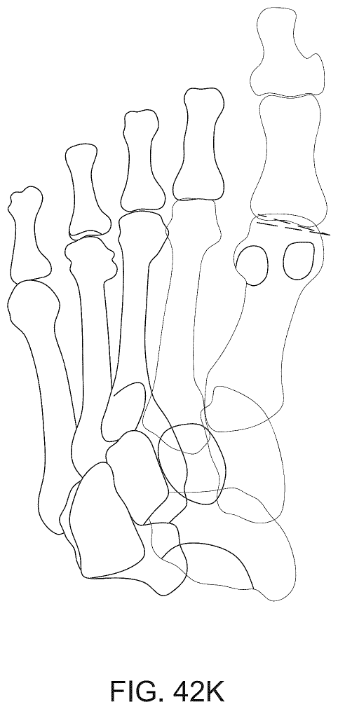

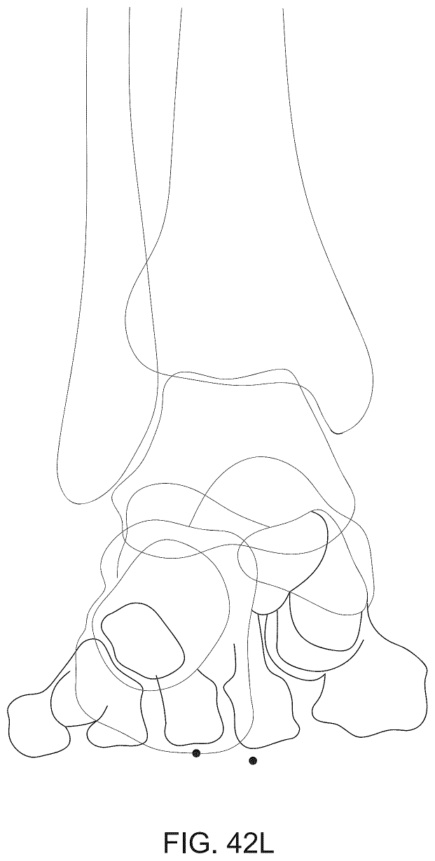

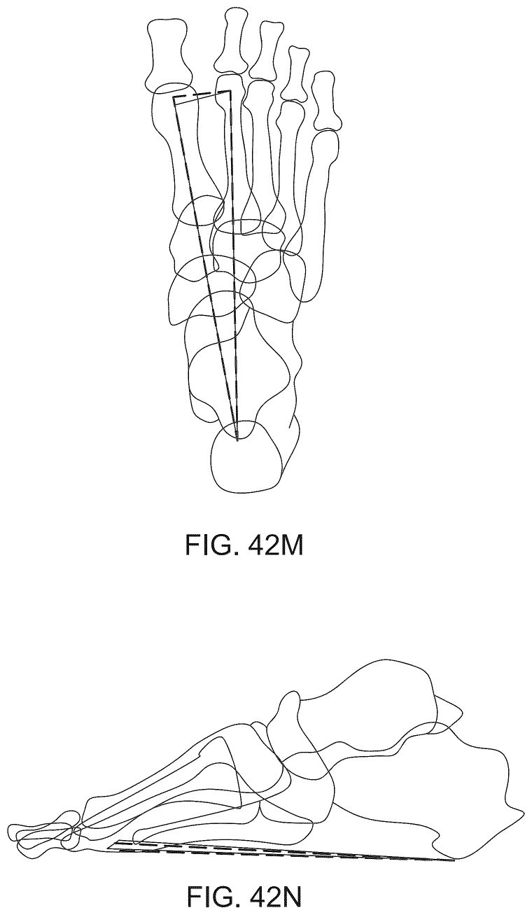

FIGS. 42A-42N illustrate example geometric measurements determined by the software relative to the foot and ankle region. For intermetatarsal angles, the 1st-2nd Intermetatarsal Angle (Axial) refers to the angle between the 1st metatarsal longitudinal axis and the 2nd metatarsal longitudinal axis measured in the axial plane, as shown in FIG. 42A.

With respect to Hallux valgus-related measurements, the Hallux Valgus Angle (Axial) refers to the angle between the 1st metatarsal longitudinal axis and the 1st proximal phalanx longitudinal axis, measured in the axial plane, as shown in FIG. 42B.

Referring to FIG. 42C, the Interphalangeal Angle (Axial) refers to the angle between the 1st proximal phalanx longitudinal axis and the 1st distal phalanx longitudinal axis measured in the axial plane.

Referring to FIG. 42D, a 1st Metatarsal Rotation refers to the angle between the 1st metatarsal distal mediolateral axis and its projection to a virtual floor plane, measured in a plane perpendicular to the 1st metatarsal longitudinal axis.

Referring to FIG. 42E, the Relative Length 1st-2nd Metatarsal refers to the distance between the distal points of the 1st and 2nd Metatarsal longitudinal axes, measured along the 2nd Metatarsal longitudinal axis.

Referring to FIG. 42F, the 1st Metatarsal Declination Angle refers to the angle between the longitudinal axis of the 1st Metatarsal and the floor level, measured in the sagittal direction.

Referring to FIG. 42G, the 1st Metatarsal Elevation refers to the distance of the lowest point of the 1st Metatarsal and the floor level.

Referring to FIG. 42H, the Plantar Gapping Angle refers to the angle between the distal joint surface of the Medial Cuneiform and the proximal joint surface of the 1st Metatarsal, measured in the direction of proximal surface of the 1st Metatarsal.

Referring to FIG. 42I, the Meary's Angle (Sagittal) refers to the angle between the talus longitudinal axis and the 1st metatarsal longitudinal axis.

Referring to FIG. 42J, the 2nd Tarsometatarsal Angle (Axial) refers to the angle between the longitudinal axes of the 2nd Metatarsal and the Intermediate cuneiform.

Referring to FIG. 42K, the Distal Metatarsal Articular Angle refers to the angle between a modulated cylinder fitted to the 1st metatarsal distal articular surface and its projection to a plane perpendicular to the 1st metatarsal.

Referring to FIG. 42L, the Hindfoot Moment Arm (Posterior) refers to the mediolateral distance between the longitudinal axis of the tibia and the most inferior point of the calcaneus.

Referring to FIG. 42M, the Triangular ratios (Axial) refers to a visualization of how the 1st metatarsal, 2nd metatarsal, and calcaneus are situated in relation to each other. A triangle is formed by the following three points: the most inferior point of the calcaneus, the centroid of the 1st metatarsal distal head, and the centroid of the 2nd metatarsal distal head.

Referring to FIG. 42N, the Triangular ratios (Sagittal) also refers to a visualization of how the 1st metatarsal, 2nd metatarsal, and calcaneus are situated in relation to each other. In this Sagittal case, a triangle is formed by the following three points: the most inferior point of the calcaneus, the most inferior point of the distal 1st metatarsal, and the most inferior point of the 2nd metatarsal.

The software can aid in automated surgical planning for a Lapidus procedure. For instance, the software performs a virtual Lapidus Arthrodesis procedure (against a digital anatomical model) to correct relevant measures of interest into their normative reference values. The measurements used can include 1st-2nd Intermetatarsal Angle (Axial) and 1st Metatarsal Rotation. Example normative reference values for these are presented in Table 5, showing a summary of measurements and normative reference values used for Smart Lapidus correction and shown concomitant procedures. In some examples, these values were computed from any number of patient WBCT images.

TABLE 5

| | | Standard deviation |
|---|---|---|
| Measurement | Average (deg.) | (SD) |
| 1^{st}-2^{nd} Intermetatarsal Angle | 11.4 | 2.0 |
| (Axial) | | |
| 1^{st}-2^{nd} Intermetatarsal Angle | 3.2 | 2.1 |
| (Sagittal) | | |
| 1^{st} Metatarsal Rotation | 2.3 | 6.2 |
| Hallux Valgus Angle (Axial) | 11.3 | 5.7 |
| Hallux Valgus Angle | 10.6 | 4.2 |
| (Sagittal) | | |
| 2^{nd} Tarsometatarsal Angle | −19.6 | 3.1 |
| (Axial) | | |
| 3^{rd} Tarsometatarsal Angle | −19.2 | 2.4 |
| (Axial) | | |

It is noted that any desired reference values can be used as the ‘normative’ reference values used by the software. The normative reference values presented above in Table 5 are similar to corresponding measurement values presented in various literature, as shown by Table 6:

TABLE 6

| Measurement | N | Average (deg.) | Reference |
|---|---|---|---|
| 1^{st}-2^{nd} Intermetatarsal | 19 | 11.3 | de Carvalho et al. |
| Angle (Axial) | | | 2022a |
| | 20 | 11.2 | de Carvalho et al. |
| | | | 2022b |
| | 100 | 11.5 | Zaidi et al. 2022 |
| 1^{st}-2^{nd} Intermetatarsal | 100 | 3.2 | Zaidi et al. 2022 |
| Angle (Sagittal) | | | |
| 1^{st} Metatarsal Rotation | 62 | 2.1 | Steadman et al. |
| | | | 2021 |
| Hallux Valgus Angle | 19 | 9.6 | de Carvalho et al. |
| (Axial) | | | 2022a |
| | 20 | 8.8 | de Carvalho et al. |
| | | | 2022b |
| Hallux Valgus Angle | 20 | 10.7 | de Carvalho et al. |
| (Sagittal) | | | 2022b |
| 2^{nd} Tarsometatarsal | | −19.6 | 3.1 |
| Angle (Axial) | | | |
| 3^{rd} Tarsometatarsal | | −19.2 | 2.4 |
| Angle (Axial) | | | |

Based on whatever normative reference values are used, if the measured 1st-2nd Intermetatarsal Angle (Axial) is smaller than the normative reference value, no correction is attempted on this angle. Moreover, the software may perform 1st metatarsal plantarflexion adjustment to take into account 1st ray shortening occurring as part of the Lapidus Arthrodesis procedure. The 1st metatarsal plantarflexion may be adjusted by setting the height of the 1st metatarsal head with respect to the 2nd metatarsal head to a level indicated by the normative reference value of the sagittal 1st-2nd Intermetatarsal Angle (see Table 5) before performing the Lapidus Arthrodesis procedure. Then, the 1st metatarsal head height may be kept constant during the procedure. As the 1st ray shortens in the Lapidus process, the software can include in the case report the following note: 1st MT shortening can be compensated with a flat wedge. With wedge, consider adjusting plantarflexion values. Thus, the user may restore the original 1st ray length by adding an implant wedge. The software can make the plan without the wedge and the user, if desiring to add one, can adjust the plan accordingly, for instance to lessen the 1st MT plantar flexion which is used to compensate for the shortening. Thus, the user may, if desired, adjust the target values for 1st-2nd Intermetatarsal Angle (Axial), 1st Metatarsal Rotation, and Plantarflexion correction, causing the software to update the plan accordingly.

The software notifies the user if Metatarsus Adductus is detected, i.e., the 2nd Tarsometatarsal Angle (Axial) is greater than a defined threshold. Ny way of non-limiting example, the defined threshold is −24°. If the user confirms to continue the planning despite the possible Metatarsus Adductus, the user then selects whether to continue with or without addressing the Metatarsus Adductus condition as part of the plan. When the option to address the Metatarsus Adductus is selected, the 2nd and 3rd rays may be rotated so that the 2nd and 3rd Tarsometatarsal Angles (Axial) match to their normative reference values (see Table 5). The Lapidus Arthrodesis procedure is performed normally after that. Note that the corrections of the 2nd and 3rd rays may be shown only for visualization purposes without a surgical procedure being defined for that. When the option to not address the Metatarsus Adductus is selected, the 1st ray is overcorrected, meaning that the 1st-2nd Intermetatarsal Angle (Axial) is corrected to its normative reference computed as if the 2nd metatarsal was in its normal position defined by the normative reference value of the 2nd Tarsometatarsal Angle (Axial). However, the overcorrection may be limited so that 1st ray bones cannot overlap with the bones on the 2nd ray.

In addition, the software performs a Hallux correction procedure to show how the 1st proximal and distal phalanges would appear if their relative placement were corrected to the normative reference values of Hallux Valgus Angles (Axial and Sagittal) (see Table 5). The Hallux correction in the software may be only for visualization purposes, without a surgical procedure being defined for that.

If the Interphalangeal angle is large, an additional AKIN procedure might be needed to properly correct the whole 1st ray. The case report from the software can show a note: Large Interphalangeal angle may indicate a need for an AKIN procedure, though the software may not define further details for the AKIN procedure.

As noted, the normative reference values used (see Table 5) can be any desired normative reference values. In examples, they are determined using an analyzer included in the software that measures a set (say, 167) of weight bearing cone-beam computed tomography (WBCT) images of normal feet (some left feet, some right feet). The corresponding software-determined reference values for other reported measurements may be presented in the case report.

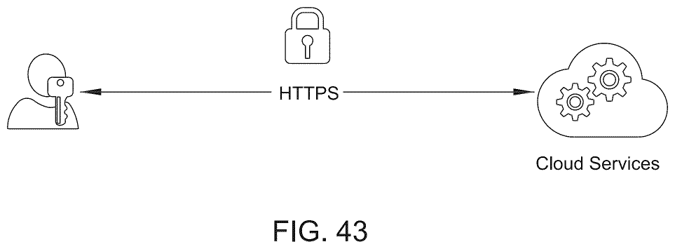

With respect to data management and software architecture, FIG. 43 depicts a conceptual diagram of secure communication (via HTTPS) between a user/client and cloud service(s) hosted on a cloud computing platform. As noted previously, the software may be provided as a cloud service with a web-based user interface. The user uses the web-based user interface to load DICOM (as one example) data to components/modules of the software executing on the cloud platform. As part of preprocessing or otherwise, 2D visualizations of input imaging data may be shown on the web interface before subsequent computations are started. Additionally, the user can provide, to the software via the interface, user-defined parameters for the software processing. Communication between the client's web browser/application and the cloud environment can be made over an HTTPS connection secured with a TLS certificate.

In the cloud environment, the software can perform computations to deidentify the DICOM data (e.g., strip personally identifiable information therefrom), calculate (by a solver) analysis models and measurements, save the results as numeric data, delete the original (uploaded) DICOM data but retain the deidentified DICOM data, use measurement results used for diagnostic purposes, and present the results to the user on the user interface, for instance by providing analysis report(s) for display/download across the HTTPS connection.

Regarding data flow and steps performed, one embodiment initially includes DICOM image handling being provided by the client workstation. For instance, DICOM data (or other image data file(s)) are read-in to a client computer system, for instance are read-in from a DICOM reader as an image series. The following information ascertained from the image data may then be displayed: Patient name, study identifier, AC number, comments; Study date time, description; Series number, date, time, modality, description. Then, when the user initiates analysis (start analysis), the user is prompted for patient evaluation information, for instance non-DICOM data related to the patient, such as height, weight, and deformities, as examples. After the user inputs this patient evaluation information, the patient evaluation information and the image series is sent to the cloud environment for analysis by the software executing in the cloud.

At the cloud, and based on the client initiating the file transfer through the secure HTTPS connection, a server/system of the cloud environment receives the image series and de-identifies the data (e.g., anonymizes it relative to any patient identifying information or other sensitive data that it might have contained), before analyzing/processing the image series in accordance with aspects described herein. The client can monitor this cloud analysis on the web-interface at the client. When the solver is ready and analysis is successful, one or more results file(s) are sent to the client through the secure HTTPS connection. The client receives the results file(s). At that point, the cloud deletes the original image series files and retains the de-identified image series. The user can save the analysis to a cloud account or other storage. Meanwhile, the cloud can delete/deallocate a software instance used to process the analysis. Analyzed cases can remain in the cloud, and existing case reports can be downloaded by the user at any time.

The Lapidus procedure used by way of example to describe features of the software is just one of multiple different surgical procedures that could potentially be employed to fix a bunion. As a result, the software could provide a feature, such as a toggle or other setting of the software, that enables a user to switch between options of (i) exploring a bunion correction procedure recommended by the software based on measurements of the anatomy (e.g., the IM angle, rotational deformity, any metatarsus adductus, etc.) that are determined/identified by the software from the user-provided image data, and (ii) being presented an interface to provide a desired quantifiable correction that the user wants to provide the patient, for instance by the user entering quantifiable corrective values for one or more measured deformities, with the software then suggesting a “best fit” procedure to achieve the desired quantifiable correction input by the user.

With respect to option (i), the software could employ a process that classifies various ranges of measurements of deformity into the specific procedures to best address those deformities. What is ‘best’ could be identified using any desired parameters, for instance based on revision percentages (lowest), invasiveness (minimal as possible), and/or any other desired factors. The ranges could be generated initially based on clinical data and then further refined on the AI based on patient outcomes. Patient outcomes (i.e., feedback) can be used to train an AI model to properly classify deformity measurement into the best procedure(s) to address them. For example, assume a patient has 22 degrees of rotational deformity, 18 degrees of IM angle deformity, and no indicated metatarsus adductus. The software could use these measurements to identify a surgical approach to address the deformities.

By way of example and not limitation or clinical practice, the software might construct the following classifications of deformity ranges for rotational deformity, IM angle deformity, and metatarsus adductus, and associate them with recommended procedures:

Rotational Deformity

| | |
|---|---|
| Range | Recommendation |

| | | |
|---|---|---|
| 0-10 | degrees | Procedure 1 |
| 10-20 | degrees | Procedure 2 |
| 20 or more | degrees | Procedure 3 |

IM Angle Deformity

| | |
|---|---|
| Range | Recommendation |

| | | |
|---|---|---|
| 0-10 | degrees | Procedure 1 |
| 10-20 | degrees | Procedure 2 |
| 20 or more | degrees | Procedure 3 |

Metatarsus Adductus

| | |
|---|---|
| Range | Recommendation |

| | | |
|---|---|---|
| 0-5 | degrees | whichever procedure best addresses |
| | | priorities 1 and 2 above |
| 5 or more | degrees | Procedure 3 |

By way of example, the procedures indicated in the above could be, but are not limited to being, a proximal rotational osteotomy of first metatarsal (‘PROMO’ procedure), a mid-shaft metatarsal osteotomy and first metatarsal shift with minimally-invasive chamfer screw fixation (‘MIS Chamfer’ procedure), and a Lapidus procedure (as described herein).

Based on the patient's example deformity measurements (22 degrees of rotational deformity, 18 degrees of IM angle deformity, and no indicated metatarsus adductus), the recommended procedure based solely on the rotational deformity is procedure 3, the recommended procedure based solely on the IM angle deformity is procedure 3, and the recommended procedure based solely on the (lack of) metatarsus adductus is one of the two. It is noted that any desired prioritization or weighting approach could be applied in situations like this where there are various (three here) deformity measurements factoring into the identification of a recommended procedure and different recommendations are produced. In one example, the rotational deformity is prioritized over the IM angle deformity and metatarsus adductus deformity. In other examples, the priority could be based on the measurements themselves, for instance if the metatarsus adductus measurement is above some threshold degrees, say 5, then the recommendation for procedure 3 might be prioritized over the recommendation resulting from either of the other two measurements.

In this example assume that the software prioritizes the recommendation of based on only the rotational deformity as the top priority. The software therefore suggests procedure 3. The software could optionally note that procedure 3 may be appropriate, as the recommendation based on the IM angle deformity.

The measurement ranges, priorities, and procedures noted above are provided strictly by way of example to illustrate aspects herein. The actual ranges, deformities considered, and prioritization scheme use could vary as desired. It is also noted that there could be fields or other inputs for the user (e.g., doctor) to offer other information to be considered by the algorithm. Examples include patient age, weight, diabetic status (yes/no), previous or concurrent procedures, arthritis condition, and/or other conditions, for instance those relevant to proper/complicated fusion/healing, etc.

With respect to option (ii) in which the user is presented an interface to provide a desired quantifiable correction and the software suggests a “best fit” procedure to achieve the correction, this might be especially useful in situations where the user, e.g., doctor or other medical professional, desires to over-correct or under-correct, for instance to facilitate another procedure that is to be performed with respect to adjacent anatomy. In this option (ii), the user is presented an interface to identify one or more desired correction(s) to patient anatomy for one or more measured deformities. The user might input the corrections themselves, and/or the user might provide desired results (measurements) of such corrections, which the software then uses to determine the correction(s) by calculating difference(s) between the starting/current patient measurement(s) and the user-provided desired results. There may also be fields for the user to input or indicate other information that is to be considered by the algorithm, such as patient age, weight, diabetic status (yes/no), previous or concurrent procedures, arthritis condition, and/or other conditions, for instance those relevant to proper/complicated fusion/healing, etc.

By way of example, assume the user identifies the following desired correction(s):
Rotational deformity desired corrected position: 4 degrees
IM Angle deformity desired corrected position: 14 degrees
Metatarsus adductus desired corrected position: 0 degrees

Assume further that the patient's pre-operative/current measurements for the above-noted deformities are 22 degrees (rotational deformity), 18 degrees (IM Angle deformity), and 0 degrees (metatarsus adductus). The software would in this case calculate the desired, or ‘total’, corrections based on the current measurements and the input desired measurements as follows:
(i) Rotational deformity:
   1. Current measured deformity: 22 degrees
   2. Desired corrected position: 4 degrees (provided by user)
   3. Total correction: 18 degrees (calculated by software)
(ii) IM angle deformity
   1. Current measured deformity: 18 degrees
   2. Desired corrected position: 14 degrees (provided by user)
   3. Total correction: 4 degrees (calculated by software)
(iii) Metatarsus adductus
   1. Current measured deformity: 0
   2. Desired corrected position: 0 degrees (provided by user)
   3. Total correction: 0 degrees (calculated by software)

With the desired corrections identified, the software could then recommend a ‘best’ procedure, for instance using the approach described above with reference to option (i), i.e., based on defined ranges ascertained from clinical data and/or based on AI and identifying for certain deformities the best procedures for correction thereof. For example, in the above scenario the software may recommend a Lapidus procedure as the best (or only) procedure capable of providing such a large (18 degrees) desired rotational correction and is also accommodating of the desired 4 degrees of angular correction. In another example, the software may recommend a PROMO procedure instead of a Lapidus. A PROMO procedure could also address an 18-degree rotational correction and the 4 degree angular correction. And, although the PROMO procedure cannot address metatarsus adductus, there is no such deformity in this instance and therefore the PROMO procedure remains a possibility. In this example, the PROMO procedure might be preferable for the patient over the Lapidus procedure on the basis that the PROMO procedure is less invasive and is expected to provide a quicker progression to post-operative weight-bearing.

As above, the measurement ranges, priorities, and procedures noted above are provided strictly by way of example to illustrate aspects herein. The actual ranges, deformities considered, and prioritization scheme use could vary as desired.

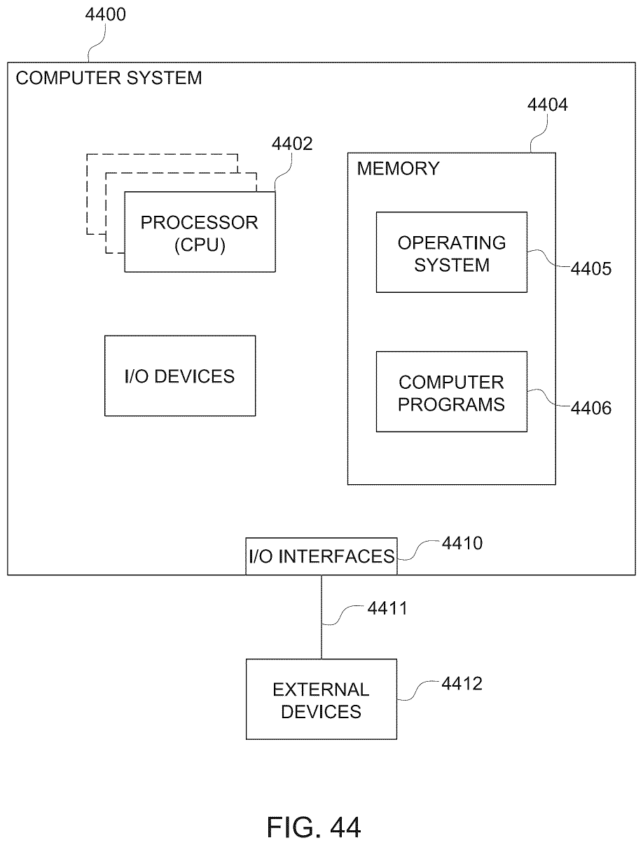

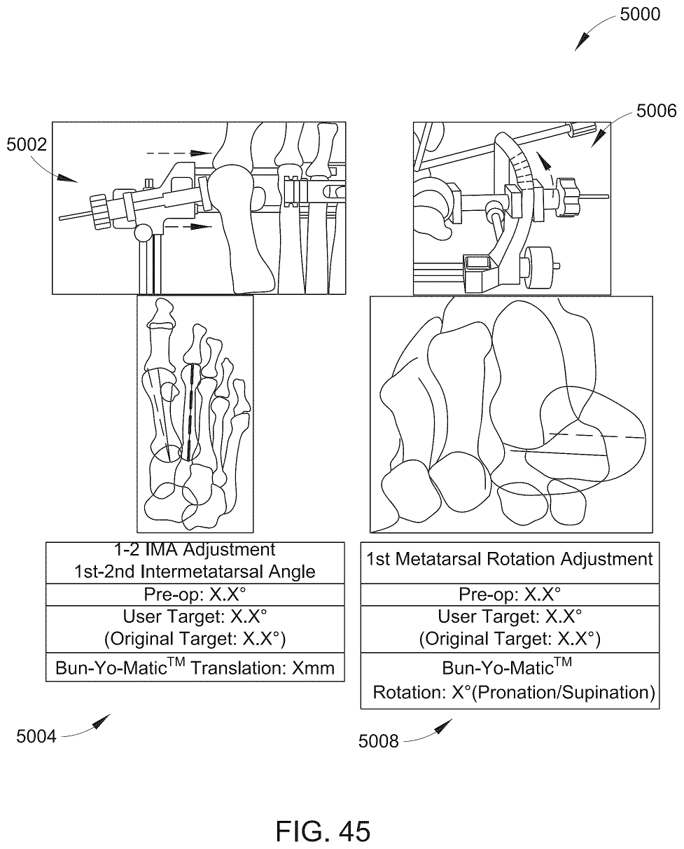

In some aspects, one or more of the processes shown and described herein, for instance with reference to FIGS. 1, and 23-24, may include an aspect (with is in addition to or in conjunction with another aspect) of generating a diagnostic report, an exemplary excerpt of which is shown in FIG. 45. The report 5000 may be generated at least in part by a computer system (e.g., 4400 shown in FIG. 44), and may be provided to a physician (whether for review/approval or for use in a procedure, as examples) in a paper or hard-copy version and/or an electronic version.

The report 5000 may include instructions, either written or visual, which correspond to one or more instruments and are configured to be implemented, performed, effected, executed, or the like by a physician to facilitate correction of one or more deformities of affected anatomy, for instance deformities identified according to aspects described herein. For example, as shown in the report 5000, an instructive image 5002 is shown adjacent a corresponding anatomical diagram 5004. The instructive image 5002 depicts a portion of the affected anatomy, for example the first metatarsal of a patient, engaged with a portion of an instrument configured to facilitate manipulation of the first and second metatarsals from a deformed position to a corrected position (with said deformed and corrected positions indicated by the dashed and solid lines, respectively, showing the long axis of the first metatarsal in the anatomical diagram 5004). The instructive image 5002 is shown to provide a visual instruction for a physician to manipulate a portion of the instrument with which the first metatarsal is engaged from a first position to a second position, thus repositioning the first metatarsal from the deformed position to the corrected position (as shown in the anatomical diagram 5004) so as to adjust the intermetatarsal angle between the long axis of the first and second metatarsals.

The report 5000 also includes an instructive image 5006 and an anatomical diagram 5008 positioned adjacent one another. The instructive image 5006 depicts a portion of the affected anatomy, for example the first metatarsal of a patient, engaged with a portion of an instrument configured to facilitate manipulation of the first metatarsal from a deformed position to a corrected position (with said deformed and corrected positions indicated by the dashed and solid lines, respectively, showing the horizontal/short/medial-lateral axis of the first metatarsal in the anatomical diagram 5008). The instructive image 5006 is shown to provide a visual instruction for a physician to manipulate a portion of the instrument with which the first metatarsal is engaged from a first position to a second position, thus repositioning the first metatarsal from the deformed position to the corrected position (as shown in the anatomical diagram 5008) so as to rotate/derotate the first metatarsal in the coronal/frontal plane.

The report 5000 may also include recommended parameters for any manipulations shown in the instructive images 5002, 5006. For example, the report 5000 may suggest that the manipulation shown in the instructive image 5002 include closing the intermetatarsal angle by a specific degree measure based on markings shown on the instrument (numbered markings, notches, etc.) corresponding to set angular manipulations. In another example, the report 5000 may suggest that the manipulation shown in the instructive image 5006 include derotating the first metatarsal by a specific degree measure based on markings shown on a different portion of the instrument shown therein (numbered markings, notches, etc.) corresponding to set rotational manipulations.

In some aspects, the report 5000 may include additional instructive images and/or corresponding anatomical diagrams showing one or more deformities and one or more corrective manipulations to reposition anatomy from deformed to corrected positions. Further, the instructive images may include various different instruments configured to facilitate such repositioning of anatomy, or may show manipulations which a physician may perform by hand.

Computer systems having memory and processor(s)/processing circuit(s) in communication with the memory can also be provided, where a computer system is configured to perform any one or more aspects of any of the above-discussed methods.

Additionally, computer program product(s) having computer readable storage medium readable by a processing circuit and storing instructions for execution by the processing circuit can be provided to perform any one or more aspects of any of the above-discussed methods.

Computer system(s) described herein could implement a surgical planning and procedures module to incorporate and/or use aspects described herein. In one or more aspects, such as module includes, in one example, various sub-modules. The sub-modules can be or include, e.g., computer readable program code (e.g., instructions) in computer readable media, e.g., persistent storage (e.g., persistent storage, such as a disk) and/or a cache (e.g., cache), as examples. The computer readable media may be part of a computer program product and may be executed by and/or using one or more computers or devices, and/or processor(s) or processing circuitry thereof, such as computer systems described herein.

Processes described herein may be performed singly or collectively by one or more computer systems, such as one or more computer systems executing surgical planning software, as an example. FIG. 44 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) or ARM Holdings plc (Cambridge, England, United Kingdom), as examples.

FIG. 44 shows a computer system 4400 in communication with external device(s) 4412. Computer system 4400 includes one or more processor(s) 4402, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 4402 can also include register(s) to be used by one or more of the functional components. Computer system 4400 also includes memory 4404, input/output (I/O) devices 4408, and I/O interfaces 4410, which may be coupled to processor(s) 4402 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 4404 can be or include main or system memory (e.g., Random Access Memory) used in the execution of program instructions, storage device(s) such as hard drive(s), flash media, or optical media as examples, and/or cache memory, as examples. Memory 4404 can include, for instance, a cache, such as a shared cache, which may be coupled to local caches (examples include L1 cache, L2 cache, etc.) of processor(s) 4402. Additionally, memory 4404 may be or include at least one computer program product having a set (e.g., at least one) of program modules, instructions, code or the like that is/are configured to carry out functions of embodiments described herein when executed by one or more processors.

Memory 4404 can store an operating system 4405 and other computer programs 4406, such as one or more computer programs/applications that execute to perform aspects described herein. Specifically, programs/applications can include computer readable program instructions that may be configured to carry out functions of embodiments of aspects described herein.

Examples of I/O devices 4408 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, and activity monitors. An I/O device may be incorporated into the computer system as shown, though in some embodiments an I/O device may be regarded as an external device (4412) coupled to the computer system through one or more I/O interfaces 4410.

Computer system 4400 may communicate with one or more external devices 4412 via one or more I/O interfaces 4410. Example external devices include a keyboard, a pointing device, a display, and/or any other devices that enable a user to interact with computer system 4400. Other example external devices include any device that enables computer system 4400 to communicate with one or more other computing systems or peripheral devices such as a printer. A network interface/adapter is an example I/O interface that enables computer system 4400 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 4410 and external devices 4412 can occur across wired and/or wireless communications link(s) 4411, such as Ethernet-based wired or wireless connections. Example wireless connections include cellular, Wi-Fi, Bluetooth®, proximity-based, near-field, or other types of wireless connections. More generally, communications link(s) 4411 may be any appropriate wireless and/or wired communication link(s) for communicating data.

Particular external device(s) 4412 may include one or more data storage devices, which may store one or more programs, one or more computer readable program instructions, and/or data, etc. Computer system 4400 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 4400 may be operational with numerous other general purpose or special purpose computing system environments or configurations. Computer system 4400 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein.

As noted, program instructions contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Thus, methods in accordance with aspects described herein can include obtaining a three-dimensional (3D) model of an anatomical region of a patient, the anatomical region comprising patient anatomical features; identifying at least one deformity of the patient anatomical features, wherein the at least one deformity is identified relative to target anatomical values for the patient anatomical features, wherein the target anatomical values comprise desired ranges into which anatomical measurements are to fall, and wherein the at least one deformity comprises at least one of a rotational deformity and an angular deformity; and determining, using the anatomical measurements, at least one planar correction to make to at least one anatomical structure of the patient.

In one or more embodiments, the at least one deformity comprises a plurality of deformities. In one or more embodiments, the plurality of deformities comprises the angular deformity. In one or more embodiments, the angular deformity comprises an inter-anatomical angle between first and second anatomical features of the patient anatomical features. In one or more embodiments, the first and second anatomical features are first and second metatarsals of the patient. In one or more embodiments, the plurality of deformities comprises the rotational deformity.

In one or more embodiments, the rotational deformity comprises a rotational deformity in a first metatarsal of the patient. In one or more embodiments, the identifying the at least one deformity comprises: selecting a geometric volume and applying the geometric volume to an anatomical feature, of the patient anatomical features, presented in the 3D model; using a property of the geometric volume, taken as a property of the anatomical feature, to determine a measurement of the anatomical measurements; and identifying a deformity, of the at least one deformity, based on the determined measurement. In one or more embodiments, the anatomical feature to which the geometric feature is applied is at least one of (i) a cross section of a bone, or (ii) an articular surface of a bone.

In one or more embodiments, the method further includes obtaining imaging data of the anatomical region of the patient, wherein the obtaining the 3D model comprises generating the 3D model from the obtained imaging data.

In one or more embodiments, the method further includes generating, based on (i) the representation of the patient anatomical features as provided by the 3D model and (ii) the determined at least one correction, a specification of patient-specific hardware to facilitate the at least one correction to make to the at least one anatomical structure, wherein the specification comprises measurements tailored to the patient based on the representation of the patient anatomical features as provided by the 3D model and on the determined at least one correction. In one or more embodiments, the patient-specific hardware comprises at least one hardware guide for guiding one or more surgical procedures to provide the at least one correction to make to the at least one anatomical structure. In one or more embodiments, the at least one hardware guide comprises at least one cut-guide for a cutting procedure.

Methods in accordance with aspects described herein can additionally or alternatively include obtaining two-dimensional (2D) imaging data of an anatomical region of a patient, the anatomical region comprising patient anatomical features; generating, using the 2D imaging data, a three-dimensional (3D) model of the anatomical region of the patient, the 3D model being specific to the patient and providing a 3D representation of the patient anatomical features; identifying one or more deformities of the patient anatomical features, wherein the one or more deformities are exhibited in the 3D model, and are identified based on the 3D model and relative to target anatomical values for the patient anatomical features, wherein the identifying comprises: obtaining anatomical measurements based on anatomical landmarks of the patient, as exhibited in the 3D model; comparing the anatomical measurements to the target anatomical values; and determining the one or more deformities based on the comparing; and determining, based on a relationship between the anatomical measurements and the target anatomical values, at least one correction to make to at least one anatomical structure of the patient.

In one or more embodiments, the method further includes identifying the anatomical landmarks, and taking the anatomical measurements based on identifying the anatomical landmarks. In one or more embodiments, the target anatomical values comprise desired ranges into which the anatomical measurements are to fall, wherein the at least one correction comprises one or more corrections to make to the at least one anatomical structure to produce updated anatomical measurements that fall within the desired ranges. In one or more embodiments, the at least one correction indicates at least one corrected position for the at least one anatomical structure.

In one or more embodiments, the method further includes generating, based on (i) the representation of the patient anatomical features as provided by the 3D model and (ii) the determined at least one correction, a specification of patient-specific hardware to facilitate the at least one correction to make to the at least one anatomical structure, wherein the specification comprises measurements tailored to the patient based on the representation of the patient anatomical features as provided by the 3D model and on the determined at least one correction. In one or more embodiments, the patient-specific hardware comprises at least one hardware guide for guiding one or more surgical procedures to provide the at least one correction to make to the at least one anatomical structure. In one or more embodiments, the at least one hardware guide comprises at least one cut-guide for a cutting procedure.

In one or more embodiments, the method further includes generating a visual simulation, wherein the visual simulation graphically presents a transition of the at least one patient anatomical structure, as represented in the 3D model, from a first position to a second position, the second position being a position that is consistent with the target anatomical values for the patient anatomical features. In one or more embodiments, the at least one correction is effected by at least one surgical activity, wherein the visual simulation further comprises one or more simulations of the at least one surgical activity relative to the at least one patient anatomical structure as represented in the 3D model, and wherein the at least one surgical activity comprises at least one of (i) at least one surgical cut, or (ii) coupling or decoupling of one or more instruments.

Methods in accordance with aspects described herein can additionally or alternatively include obtaining two-dimensional (2D) imaging data of an anatomical region of a patient, the anatomical region comprising patient anatomical features; and generating, using the 2D imaging data, a three-dimensional (3D) model of the anatomical region of the patient, the 3D model being specific to the patient and providing a 3D representation of the patient anatomical features, wherein the generating comprises: performing anatomical context processing, the anatomical context processing comprising annotating, using an artificial-intelligence (AI) model, contours, edges, or surfaces of anatomical structures, of the anatomical region, presented in the 2D imaging data; and performing 2D-to-3D reconstruction that optimizes orientation, placement, and scale of 3D digital volumes modeling the anatomical structures to yield, as the 3D model, a 3D anatomical representation of the anatomical region.

In one or more embodiments, the 2D imaging data comprises two or more images or different views of the anatomical region. In one or more embodiments, the images or different views are radiographs. In one or more embodiments, the AI model performs image segmentation in two dimensions to identify locations for the contours, edges, or surfaces of the anatomical structures. In one or more embodiments, the image segmentation produces 2D segmentations, and wherein the method further comprises processing the 2D segmentations into a set of 2D points based on determining, for each of the anatomical structures, landmarks of the anatomical structure, the landmarks of the anatomical structure comprising a centroid of the anatomical structure.

In one or more embodiments, the annotating provides annotations that include outlines of peripheries of the anatomical structures. In one or more embodiments, the anatomical structures comprise bones, and wherein the annotations show the bones contoured on one or more 2D image planes. In one or more embodiments, the method further includes displaying, on a display, the outlines of the peripheries of the anatomical structures, wherein the outlines are presented with varying graphical properties to facilitate identification and distinction between the anatomical structures.

In one or more embodiments, the 2D-to-3D reconstruction comprises fitting together, in 3D space, planes of 2D imaging data depicting the anatomical structures, the fitting comprising iteratively transforming the planes based on a comparison anatomical model to alter how the planes sit relative to each other, wherein the fitting provides the 3D anatomical representation of the anatomical region. In one or more embodiments, the 2D imaging data informs, via image processing techniques including one or more of edge detection and gradient spikes, plane transformations to ensure consistency between the plane transformations and what is depicted by the 2D imaging data. In one or more embodiments, the comparison anatomical model comprises a 3D model having structures with comparison properties for the anatomical region, wherein the comparison properties comprise at least one of sizes, shapes, or orientations. In one or more embodiments, the comparison anatomical model is provided with indications of how the comparison properties can vary based on a selected population of anatomical samples. In one or more embodiments, the indications are encoded into the comparison anatomical model.

In one or more embodiments, the method further includes building the AI model to process incoming 2D imaging data and provide annotations thereto, the building comprising training the AI model on 2D segmented images that are pre-annotated as to landmarks of anatomy. In one or more embodiments, the landmarks include bone centroids and proximal and distal ends of bones. In one or more embodiments, the method further includes building a training dataset of samples, the samples comprising the 2D segmented images. In one or more embodiments, the building the training dataset comprises obtaining bilateral images and splitting the bilateral images into unilateral images that form some of the samples. In one or more embodiments, the building the training dataset comprises producing additional samples for the training dataset by applying at least one of rotations, flips, translations, and other image manipulations to existing samples of the training data set.

Methods in accordance with aspects described herein can additionally or alternatively include obtaining two-dimensional (2D) imaging data of an anatomical region of a patient, the anatomical region comprising patient anatomical features; generating, using the 2D imaging data, a three-dimensional (3D) model of the anatomical region of the patient, the 3D model being specific to the patient and providing a 3D representation of the patient anatomical features; identifying one or more deformities of the patient anatomical features, wherein the one or more deformities are exhibited in the 3D model, and are identified based on the 3D model and relative to target anatomical values for the patient anatomical features, wherein the identifying comprises: obtaining anatomical measurements based on anatomical landmarks of the patient, as exhibited in the 3D model; comparing the anatomical measurements to the target anatomical values; and determining the one or more deformities based on the comparing; determining, based on a relationship between the anatomical measurements and the target anatomical values, at least one correction to make to at least one anatomical structure of the patient; and generating a report comprising instructions for engaging a surgical instrument with the at least one anatomical structure of the patient and manipulating said surgical instrument to perform the at least one correction to the at least one anatomical structure of the patient.

Computer systems having a memory and a processing circuit in communication with the memory, and that are configured to perform any of the foregoing methods, and computer program products having a computer readable storage medium readable by a processing circuit and storing instructions for execution by the processing circuit to perform any of the foregoing methods, are also provided.

Although various embodiments are described above, these are only examples.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A method comprising:
obtaining two-dimensional (2D) imaging data of an anatomical region of a patient, the anatomical region comprising patient anatomical features;
generating, using the 2D imaging data, a three-dimensional (3D) model of the anatomical region of the patient, the 3D model being specific to the patient and providing a 3D representation of the patient anatomical features;
identifying one or more deformities of the patient anatomical features, wherein the one or more deformities are exhibited in the 3D model, and are identified based on the 3D model and relative to target anatomical values for the patient anatomical features, wherein the identifying comprises:
obtaining anatomical measurements based on anatomical landmarks of the patient, as exhibited in the 3D model;
comparing the anatomical measurements to the target anatomical values; and
determining the one or more deformities based on the comparing; and
determining, based on a relationship between the anatomical measurements and the target anatomical values, at least one correction to make to at least one anatomical structure of the patient.

2. The method of claim 1, further comprising identifying the anatomical landmarks, and taking the anatomical measurements based on identifying the anatomical landmarks.

3. The method of claim 1, wherein the target anatomical values comprise desired ranges into which the anatomical measurements are to fall, wherein the at least one correction comprises one or more corrections to make to the at least one anatomical structure to produce updated anatomical measurements that fall within the desired ranges.

4. The method of claim 1, wherein the at least one correction indicates at least one corrected position for the at least one anatomical structure.

5. The method of claim 1, further comprising generating, based on (i) the representation of the patient anatomical features as provided by the 3D model and (ii) the determined at least one correction, a specification of patient-specific hardware to facilitate the at least one correction to make to the at least one anatomical structure, wherein the specification comprises measurements tailored to the patient based on the representation of the patient anatomical features as provided by the 3D model and on the determined at least one correction.

6. The method of claim 5, wherein the patient-specific hardware comprises at least one hardware guide for guiding one or more surgical procedures to provide the at least one correction to make to the at least one anatomical structure.

7. The method of claim 6, wherein the at least one hardware guide comprises at least one cut-guide for a cutting procedure.

8. The method of claim 1, further comprising generating a visual simulation, wherein the visual simulation graphically presents a transition of the at least one patient anatomical structure, as represented in the 3D model, from a first position to a second position, the second position being a position that is consistent with the target anatomical values for the patient anatomical features.

9. The method of claim 8, wherein the at least one correction is effected by at least one surgical activity, wherein the visual simulation further comprises one or more simulations of the at least one surgical activity relative to the at least one patient anatomical structure as represented in the 3D model, and wherein the at least one surgical activity comprises at least one of (i) at least one surgical cut, or (ii) coupling or decoupling of one or more instruments.

10. A computer system comprising:
a memory; and
a processing circuit in communication with the memory, wherein the computer system is configured to perform a method comprising:
obtaining two-dimensional (2D) imaging data of an anatomical region of a patient, the anatomical region comprising patient anatomical features;
generating, using the 2D imaging data, a three-dimensional (3D) model of the anatomical region of the patient, the 3D model being specific to the patient and providing a 3D representation of the patient anatomical features;
identifying one or more deformities of the patient anatomical features, wherein the one or more deformities are exhibited in the 3D model, and are identified based on the 3D model and relative to target anatomical values for the patient anatomical features, wherein the identifying comprises:
obtaining anatomical measurements based on anatomical landmarks of the patient, as exhibited in the 3D model;
comparing the anatomical measurements to the target anatomical values; and
determining the one or more deformities based on the comparing; and
determining, based on a relationship between the anatomical measurements and the target anatomical values, at least one correction to make to at least one anatomical structure of the patient.

11. The computer system of claim 10, wherein the at least one correction indicates at least one corrected position for the at least one anatomical structure.

12. The computer system of claim 10, wherein the method further comprises generating, based on (i) the representation of the patient anatomical features as provided by the 3D model and (ii) the determined at least one correction, a specification of patient-specific hardware to facilitate the at least one correction to make to the at least one anatomical structure, wherein the specification comprises measurements tailored to the patient based on the representation of the patient anatomical features as provided by the 3D model and on the determined at least one correction.

13. The computer system of claim 12, wherein the patient-specific hardware comprises at least one hardware guide for guiding one or more surgical procedures to provide the at least one correction to make to the at least one anatomical structure.

14. The computer system of claim 13, wherein the at least one hardware guide comprises at least one cut-guide for a cutting procedure.

15. The computer system of claim 10, wherein the method further comprises generating a visual simulation, wherein the visual simulation graphically presents a transition of the at least one patient anatomical structure, as represented in the 3D model, from a first position to a second position, the second position being a position that is consistent with the target anatomical values for the patient anatomical features, wherein the at least one correction is effected by at least one surgical activity, wherein the visual simulation further comprises one or more simulations of the at least one surgical activity relative to the at least one patient anatomical structure as represented in the 3D model, and wherein the at least one surgical activity comprises at least one of (i) at least one surgical cut, or (ii) coupling or decoupling of one or more instruments.

16. A computer program product comprising:
a computer readable storage medium readable by a processing circuit and storing instructions for execution by the processing circuit to perform a method comprising:
obtaining two-dimensional (2D) imaging data of an anatomical region of a patient, the anatomical region comprising patient anatomical features;
generating, using the 2D imaging data, a three-dimensional (3D) model of the anatomical region of the patient, the 3D model being specific to the patient and providing a 3D representation of the patient anatomical features;
identifying one or more deformities of the patient anatomical features, wherein the one or more deformities are exhibited in the 3D model, and are identified based on the 3D model and relative to target anatomical values for the patient anatomical features, wherein the identifying comprises:
obtaining anatomical measurements based on anatomical landmarks of the patient, as exhibited in the 3D model;
comparing the anatomical measurements to the target anatomical values; and
determining the one or more deformities based on the comparing; and
determining, based on a relationship between the anatomical measurements and the target anatomical values, at least one correction to make to at least one anatomical structure of the patient.

17. The computer program product of claim 16, wherein the at least one correction indicates at least one corrected position for the at least one anatomical structure.

18. The computer program product of claim 16, wherein the method further comprises generating, based on (i) the representation of the patient anatomical features as provided by the 3D model and (ii) the determined at least one correction, a specification of patient-specific hardware to facilitate the at least one correction to make to the at least one anatomical structure, wherein the specification comprises measurements tailored to the patient based on the representation of the patient anatomical features as provided by the 3D model and on the determined at least one correction, wherein the patient-specific hardware comprises at least one hardware guide for guiding one or more surgical procedures to provide the at least one correction to make to the at least one anatomical structure.

19. The computer program product of claim 18, wherein the at least one hardware guide comprises at least one cut-guide for a cutting procedure.

20. The computer program product of claim 16, wherein the method further comprises generating a visual simulation, wherein the visual simulation graphically presents a transition of the at least one patient anatomical structure, as represented in the 3D model, from a first position to a second position, the second position being a position that is consistent with the target anatomical values for the patient anatomical features, wherein the at least one correction is effected by at least one surgical activity, wherein the visual simulation further comprises one or more simulations of the at least one surgical activity relative to the at least one patient anatomical structure as represented in the 3D model, and wherein the at least one surgical activity comprises at least one of (i) at least one surgical cut, or (ii) coupling or decoupling of one or more instruments.
